(19) United States
(12) Patent Application Publication
(10) Pub. No.: US 2026/0092248 A1
(43) Pub. Date: Apr. 2, 2026

(54) BIOREACTORS OPERABLE IN STATIC AND DYNAMIC MODES AND METHODS OF USE

(71) Applicant: LIFE TECHNOLOGIES CORPORATION, Carlsbad, CA (US)

(72) Inventors: Michael Goodwin, Logan, UT (US); Xavier De Mollerat Du Jeu, Encinitas, CA (US); Tristan Wise, Nibley, UT (US); Tony Hsiao, Providence, UT (US); Andrew Campbell, Tonawanda, NY (US); Paul Thacker, Lewiston, UT (US); Michael Derrick, Logan, UT (US); Jacob Kaiser, Smithfield, UT (US)

(21) Appl. No.: 19/111,653

(22) PCT Filed: Sep. 14, 2023
(86) PCT No.: PCT/US2023/074211

§ 371 Date: Mar. 13, 2025

Related U.S. Application Data
(60) Provisional application No. 63/406,967, filed on Sep. 15, 2022.

(51) Int. Cl. C12M 1/36; C12M 1/00; C12M 1/06; C12M 3/00

(52) U.S. Cl. CPC C12M 41/48; C12M 23/44; C12M 27/08; C12M 29/06

(57) ABSTRACT

The application describes bioreactors comprising gas permeable membranes disposed on the housing covering at least partially a transfer opening. The bioreactor is also provided with a mixing element and ports adapted to receive sensors measuring values of parameters related to the process implemented in the bioreactor. The bioreactor comprises at least one opening in one of the walls. The at least one opening is covered with a gas permeable membrane.

### CROSS-REFERENCE TO RELATED APPLICATIONS

This application claims the benefit of U.S. Provisional Application No. 63/406,967, filed on Sep. 15, 2022, which is incorporated herein by specific reference.

### BACKGROUND OF THE INVENTION

### 1. the Field of the Invention

The present invention relates to bioreactors that can operate in multiple cell culturing modes and bioreactor orientations to maximize and optimize cell expansion, cell preservation, and cell processing within a single bioreactor vessel. For example, bioreactors of the present disclosure can operate in a static mode, where no mechanical mixing occurs, and a dynamic mode, where any degree of mechanical mixing occurs, and/or alternating modes of operation and in vertical, horizontal, or angled orientations within each cell culturing mode.

### 2. the Relevant Technology

The expansion of biological cells within a bioreactor requires critical control over a number of different process parameters. For example, as cells expand, they absorb oxygen from the surrounding media and release CO₂. The concentration of oxygen and CO₂ within the media must be carefully monitored and regulated to ensure viability and optimal expansion of the cells. Other processing parameters that are commonly monitored and controlled include pH, temperature, and cell density. To help maintain proper control over the various processing parameters as cell populations increase, cells are typically cultured by sequentially transferring them to increasingly larger bioreactors. For example, a cellular seed culture may initially start by being cultured in a small flask, which is moved on a shaker table to keep the cell suspension uniformly mixed. Once the cell density reaches a predetermined value, the cell suspension is transferred to a larger bench top bioreactor where the suspension is combined with additional media. To maintain proper mixing and oxygenation of the larger volume of cell suspension within the bench top bioreactor, the bench top bioreactor is commonly fitted with an internal impeller for mechanical mixing and a sparger for delivering gas into the compartment. In turn, once the cell density increases to again reach a predetermined value, the cell suspension can be transferred to a larger production bioreactor for further expansion with additional media.

Although the time-tested method of sequentially transferring and expanding cell populations to increasing larger bioreactors to achieve desired cell production is effective, it has a number of shortcomings. For example, transferring cells between different bioreactors is time consuming and labor intensive. Furthermore, the cell suspension must be constantly maintained sterile, often under strict verification requirements. Transferring the cell suspension between different bioreactors increases the risk of contamination. In addition, using multiple different bioreactors to expand a single cell batch is expensive in the cost, operation, storage and maintaining of multiple different types, sizes and/or designs of bioreactors. Other shortcomings also exist.

Accordingly, what is needed in the art are new bioreactors and related methods of operation that minimize some or all of the above shortcomings.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a front perspective view of a bioreactor.

FIG. 2 is a rear perspective view of the bioreactor shown in FIG. 1.

FIG. 3 is a partially exploded view of the bioreactor shown in FIG. 1.

FIG. 4 is a perspective view of the bioreactor shown in FIG. 1 with the encircling sidewall thereof removed.

FIG. 5 is a subassembly of the bioreactor shown in FIG. 1 showing the drive shaft and mixing elements thereon.

FIG. 6 is an enlarged cross-sectional view of the terminal end of the drive shaft shown in FIG. 5 engaging with the steady support.

FIG. 7 is a perspective view of a plurality of the bioreactors shown in FIG. 1 stacked on a shelf, such as within an incubator.

FIG. 8 is a perspective view of an alternative embodiment of the drive shaft shown in FIG. 5 with a free-floating terminal end and alternative spargers that can be used in the bioreactor.

FIG. 9 is a partially exploded perspective view of an alternative embodiment of the bioreactor shown in FIG. 1 wherein a second transfer opening is formed on the bottom end wall and is covered by a second gas permeable membrane.

FIG. 10 is a perspective view of an alternative embodiment of the drive shaft shown in FIG. 5 which includes adjacent drive shaft portions each having a helical configuration.

FIG. 11 is a left side perspective view of an alternative embodiment of a bioreactor system.

FIG. 12 is a right side perspective view of the bioreactor system shown in FIG. 11.

FIG. 13 is an enlarged perspective view of the bottom end wall of the bioreactor shown in FIG. 11.

FIG. 14 is an enlarged perspective view of the top end wall of the bioreactor shown in FIG. 11.

FIG. 15 is an enlarged bottom perspective view of the top end wall shown in FIG. 14.

FIG. 16 is a top perspective view of the heater stand shown in FIG. 11.

FIG. 17 is a bottom perspective view of the heater stand shown in FIG. 16.

FIG. 18 is an exploded view of an alternative embodiment of the support housing shown in FIG. 11.

FIG. 19 is a perspective view of another alternative embodiment of a bioreactor system.

FIG. 20 is a perspective view of a heating jacket that can be used with the bioreactors disclosed herein.

FIG. 21 is perspective view of a bioreactor in accordance with example embodiments.

FIG. 22 is a top view of the bottom of top cap of the bioreactor of FIG. 21.

FIG. 23A depicts an impeller assembly in accordance with example embodiments.

FIG. 23B is a cross sectional view of an impeller mounting hub in accordance with example embodiments.

FIGS. 24A-B depict top and perspective views of a bioreactor base in accordance with example embodiments.

FIGS. 25A-B depict a perspective view of a bioreactor and a top view of a bioreactor base in accordance with example embodiments.

FIG. 26 depicts a sparger in accordance with example embodiments.

FIG. 27 depicts a bioreactor base in accordance with example embodiments.

FIGS. 28A-C depict sensor and port packs in accordance with example embodiments.

FIGS. 29A-B depict front and back views of a reactor pod in accordance with example embodiments.

FIG. 30 is a bioreactor pod wall in accordance with example embodiments.

FIGS. 31A-D depict front view, back view and cross-sectional views of a sensor pack in accordance with example embodiments.

FIG. 32 is a top end wall of a bioreactor in accordance with example embodiments.

FIGS. 33A-E depict a series of impellers in accordance with example embodiments.

FIGS. 34A-C depict perspective, cross sectional and partial top views of an impeller mounting hub in accordance with example embodiments.

FIGS. 35A-B depict a perspective and cross-sectional views of an impeller assembly in accordance with example embodiments.

FIG. 36 depicts an autologous cell therapy system and process flow, including one or more dual mode bioreactors or bioreactor pods in accordance with example embodiments.

### DETAILED DESCRIPTION OF THE PREFERRED EMBODIMENTS

Before describing the present disclosure in detail, it is to be understood that this disclosure is not limited to particularly exemplified apparatus, systems, methods, or process parameters that may, of course, vary. It is also to be understood that the terminology used herein is only for the purpose of describing particular exemplary embodiments of the present disclosure and is not intended to limit the scope of the disclosure in any manner.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “port” includes one, two, or more ports.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure or claims.

Where possible, like numbering of elements have been used in various figures. Furthermore, multiple instances of an element and or sub-elements of a parent element may each include separate letters appended to the element number. For example, two instances of a particular element “10” may be labeled as “10A” and “10B”. In that case, the element label may be used without an appended letter (e.g., “10”) to generally refer to all instances of the element or any one of the elements. Element labels including an appended letter (e.g., “10A”) can be used to refer to a specific instance of the element or to distinguish or draw attention to multiple uses of the element. Furthermore, an element label with an appended letter can be used to designate an alternative design, structure, function, implementation, and/or embodiment of an element. For example, two alternative exemplary embodiments of a particular element may be labeled as “10A” and “10B”. In that case, the element label may be used without an appended letter (e.g., “10”) to generally refer to all instances of the alternative embodiments or any one of the alternative embodiments.

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “connected,” and the like do not necessarily imply direct contact between the two or more elements.

As used herein, the term “gas permeable membrane” is a layer (e.g., solid layer or non-fluidic layer) that allows gas to pass through. More specifically, a “gas permeable membrane” can be a membrane that permits various gas molecules, including oxygen, carbon dioxide, and nitrogen, to pass through the membrane as a result of a pressure, partial pressure, or concentration differential across the membrane. However, gas permeability of the membrane does not permit a gas stream or visible gas bubbles to pass through. In exemplary embodiments, the gas permeability of the membrane at 23 degrees Celsius and 1 bar can be in a range between 500 mL/(m²*day) and 25,000 mL/(m²*day) with 5,000 mL/(m²*day) and 10,000 mL/(m²*day) being more preferred. The gas permeability is also typically lower than 75,000 mL/(m²*day), 100,000 mL/(m²*day), 125,000 mL/(m²*day), or 150,000 mL/(m²*day). Gases that these membranes may be permeable to include, for example, O₂, CO₂, and N₂. Gas permeable silicone (e.g., dimethyl silicone) membranes approximately 0.005 to 0.007 inches thick may be used and are referred to in U.S. Pat. No. 9,567,565, which is hereby incorporated by specific reference. Example gas permeable membranes include those in the G-REX® series, which may be obtained from Wilson Wolf Corporation, 33 5th Ave NW, Saint Paul, MN 55112 (see, e.g., P/Ns 85500S-CS and 81100S). Other examples of gas permeable membranes and devices containing them are gas permeable plates available from Coy Lab Products (see cat. no. 8602000). These plates allow for the control of O₂ levels that cells are contacted with in incubators. Specifications of these plates are as follows: 25 μm polymer film which allows high gas transfer rate while retaining liquid, O₂ permeability greater than 9000 cm³/M², CO₂ permeability greater than 7000 cm³/M².

The terms “expanded” and “expansion,” as used herein, refer to cellular reproduction. For example, if the number of cells in a culture medium increases from 1,000 cells to 4,000 cells, the cells would be expanded four-fold. Assuming 100% of the cells in the culture medium are both reproducing and reproducing at the same rate, this amount of expansion would occur after two cell divisions. In many instances herein, the terms “cultured” and “expanded” are used interchangeably.

The term “activation,” as used herein, refers to the state of a cell following sufficient cell surface moiety ligation to induce a measurable morphological, phenotypic, and/or functional change. Within the context of T cells, such activation may be the state of a T cell that has been sufficiently stimulated to induce cellular proliferation. Activation of a T cell may also induce cytokine production and/or secretion, and up-or down-regulation of expression of cell surface molecules, such as receptors or adhesion molecules, or up- or down-regulation of secretion of certain molecules, and performance of regulatory or cytolytic effector functions. Within the context of other cells, this term infers either up- or down-regulation of a particular physico-chemical process.

In some instances, stimulation may comprise a primary response induced by ligation of a cell surface moiety. For example, in the context of receptors, such stimulation may entail the ligation of a receptor and a subsequent signal transduction event. In some instances, expansion of T cells, for example, may comprise stimulating of these T cells. With respect to stimulation of a T cell, such stimulation may refer to the ligation of a T cell surface moiety that in embodiments subsequently induces a signal transduction event, such as binding the TCR/CD3 complex. In some instances, the stimulation event may activate a cell and up- or down-regulate expression of cell surface molecules such as receptors or adhesion molecules, or up- or down-regulate secretion of a molecule, such as down-regulation of Tumor Growth Factor beta (TGF-β) or up-regulation of IL-2, IFN-γ etc. In some instances, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

The term “stimulatory agent”, as used herein, refers to a molecule that binds to one or more cell type and induces a cellular response. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), or the like. In embodiments, in the context of T cell stimulation, antibodies are used as a prototypical example of such an agent.

Antibodies for use in methods set out herein may be of any species, class or subtype providing that such antibodies can react with the target of interest, e.g., CD3, the TCR, or CD28, as appropriate. Thus “antibodies” for use in methods set out herein include:
(a) any of the various classes or sub-classes of immunoglobulin (e.g., IgG, IgA, IgM, IgD or IgE derived from any animal, e.g., any of the animals conventionally used, e.g., sheep, rabbits, goats, mice, camelids, or egg yolk),
(b) monoclonal or polyclonal antibodies,
(c) intact antibodies or fragments of antibodies, monoclonal or polyclonal, the fragments being those which contain the binding region of the antibody, e.g., fragments devoid of the Fc portion (e.g., Fab, Fab′, F(ab′)2, scFv, VHH, or other single domain antibodies), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains.
(d) antibodies produced or modified by recombinant DNA or other synthetic techniques, including monoclonal antibodies, fragments of antibodies, “humanized antibodies”, chimeric antibodies, or synthetically made or altered antibody-like structures.

Also included are functional derivatives or “equivalents” of antibodies e.g., single chain antibodies, CDR-grafted antibodies etc. A single chain antibody (SCA) may be defined as a genetically engineered molecule containing the variable region of the light chain, the variable region of the heavy chain, linked by a suitable polypeptide linker as a fused single chain molecule.

“Chimeric antigen receptor” or “CAR” or “CARs” as used herein refers to engineered receptors, which graft an antigen specificity onto cells (for example T cells such as naïve T cells, central memory T cells, effector memory T cells or any combination thereof). CARs are also known as artificial T cell receptors, chimeric T cell receptors or chimeric immunoreceptors. In embodiments, a CAR comprises one or more antigen-specific targeting domains, an extracellular domain, a transmembrane domain, one or more co-stimulatory domains, and an intracellular signaling domain. In embodiments, if the CAR targets two different antigens, the antigen-specific targeting domains may be arranged in tandem. In embodiments, if the CAR targets two different antigens, the antigen-specific targeting domains may be arranged in tandem and separated by linker sequences.

CARs are engineered receptors, which graft an arbitrary specificity onto an immune cell (e.g., a T cell, such as an activated T cell). These receptors are used to graft the specificity of a monoclonal antibody onto immune cells; with transfer of their coding sequence facilitated by retroviral vectors. The receptors are called chimeric because they are composed of parts from different sources. CARs may be used as a therapy for cancer through adoptive cell transfer. T cells are removed from a patient and modified so they express receptors specific to the patient's particular cancer. The T cells, which recognize and kill the cancer cells, are reintroduced into the patient. In embodiments, modification of T cells sourced from donors other than the patient may be used to treat the patient.

Using adoptive transfer of T cells expressing chimeric antigen receptors, CAR-modified T cells can be engineered to target any tumor-associated antigen. Following the collection of a patient's T cells, the cells are genetically engineered to express CARs specifically directed towards antigens on the patient's tumor cells before being infused back into the patient.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

Various aspects of the present devices, systems, and methods may be illustrated with reference to one or more exemplary embodiments. As used herein, the terms “exemplary,” “embodiment,” and “exemplary embodiment” mean “serving as an example, instance, or illustration,” and should not necessarily be construed as required or as preferred or advantageous over other embodiments disclosed herein.

The present invention relates to bioreactors used for expanding cells found in a suspension that is dispensed into or formed within the bioreactors. In exemplary embodiments, the cells are human cells, such as T cells, used in cell therapy. The bioreactors can be operated in both a static mode, wherein the mixer of the bioreactor is stationary and/or no mixing of the suspension is implemented during a specific growth phase (e.g., first phase, post electroporation, or other phase where cells are more susceptible to damage) and a dynamic mode, wherein minimal, continuous, substantially continuous, low, or high mixing, such as rotations per minute (RPM) mixing, can be used to mix the suspension during a second growth phase.

Bioreactors set out herein may be operated by rotating a mixing element at various RPMs. The number of RPMs used will typically vary with the type/configuration of the mixing element and the type and condition of the cells in the bioreactor. This is so, in part, because some cells are more resistant to mechanical shearing than other cells. In general, mixing element RPMs, such as impellers, will often vary from about 40 to about 1,500 RPMs (e.g., from about 40 to about 1,000, from about 40 to about 800, from about 40 to about 600, from about 40 to about 400, from about 40 to about 300, from about 40 to about 200, from about 100 to about 1,000, from about 100 to about 800, from about 100 to about 600, from about 100 to about 400, from about 100 to about 300, from about 60 to about 1,000, from about 60 to about 300, etc., RPMs). For more sensitive process conditions or when cell types or conditions are fragile, for example after electroporation, the bioreactor and mixer can be operated in dynamic mode at lower RPMs of less than 40 RPMs or less than 10 RPMs.

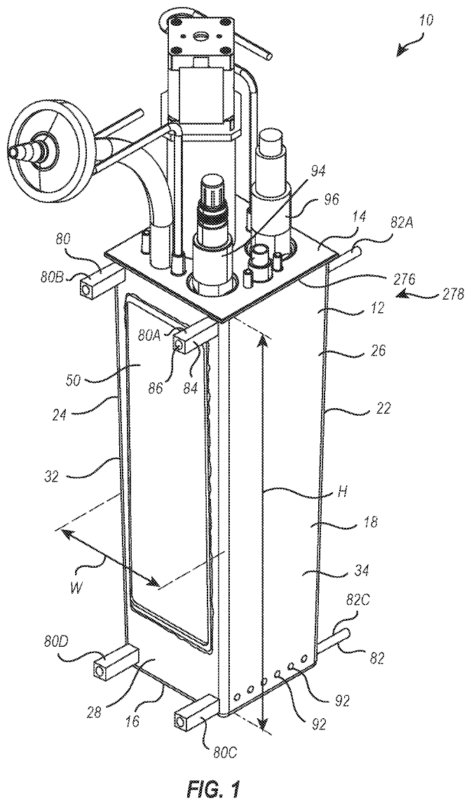

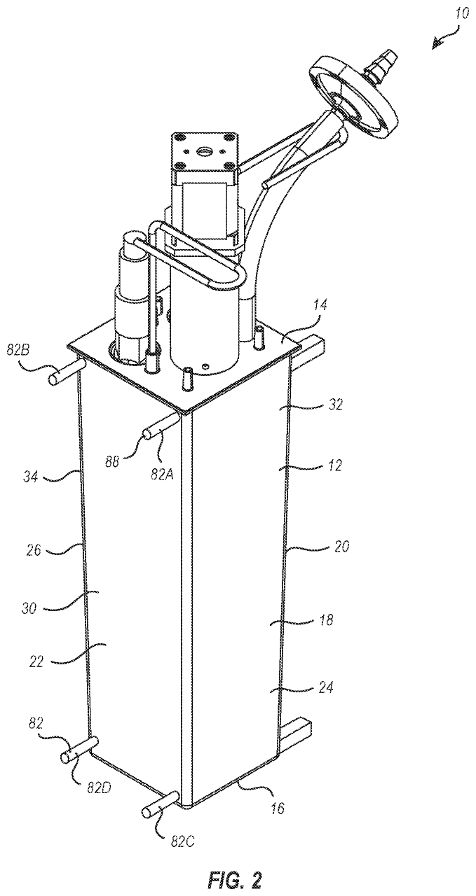

Depicted in FIGS. 1 and 2 is one embodiment of a bioreactor 10 incorporating features of the present disclosure. Bioreactor 10 includes a housing 12 having a top end wall 14, an opposing bottom end wall 16, and an encircling sidewall 18 extending therebetween. Encircling sidewall 18 has a front face 20, an opposing back face 22, and opposing side faces 24 and 26 that each extend between top end wall 14 and bottom end wall 16.

In the depicted embodiment, encircling sidewall 18 has a rectangular or square transverse cross section. However, in other embodiments, encircling sidewall 18 can have other transverse cross section configurations such as circular, elliptical, or polygonal. More specifically, encircling sidewall 18 is depicted as comprising a front wall 28 that includes front face 20, a back wall 30 that includes back face 22, and opposing sidewalls 32 and 34 that extend between front wall 28 and back wall 30. Sidewalls 32 and 34 include side faces 24 and 26, respectively.

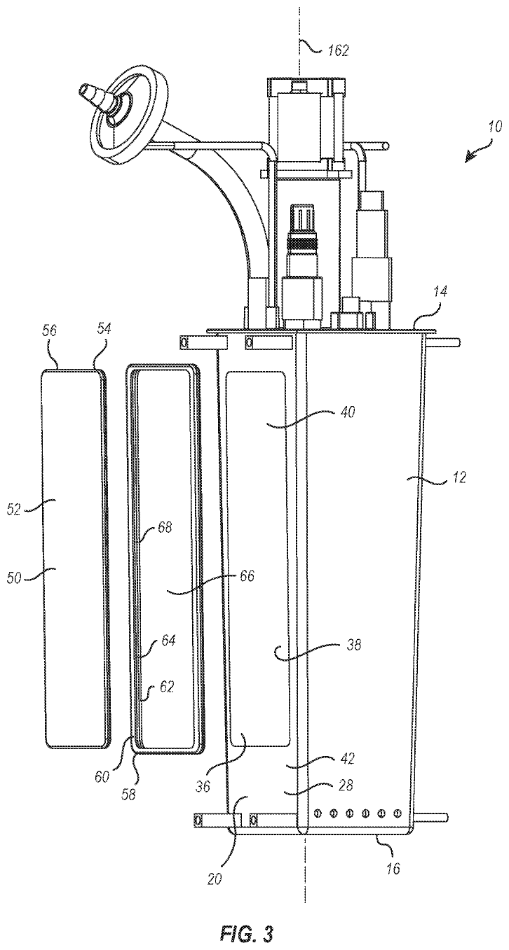

Turning to FIG. 3, housing 12 has an interior surface 36 that bounds a compartment 38. To facilitate proper operation, compartment 38 is specifically designed to be relatively small. For example, compartment 38 typically has a volume of at least or less than 50 milliliters, 100 milliliters, 250 milliliters, 500 milliliters, 1 liter, 5 liters, 10 liters, 20 liters, 30 liters, 40 liters, 50 liters, or in a range between any two of the foregoing. For example, compartment 38 commonly has a volume in a range between 250 milliliters and 50 liters with between 1 liter and 20 liters or between 1 liter and 10 liters being more common. Other volumes can also be used. In one embodiment, housing 12 and the walls thereof are made from a material that is impermeable to gas and liquid, such as media.

Furthermore, housing 12 and the walls thereof are generally rigid. For example, in one exemplary embodiment, housing 12 is sufficiently rigid that it does not bow, flex and/or expand when compartment 38 is filled with liquid, such a water or media. Housing 12 is commonly made from a plastic such as polycarbonate, polyolefins, polyester, polystyrene, and polyacrylics and can be produced through a molding process such as injection molding, extrusion, blow molding, 3d printing (additive manufacturing), rotational molding, or any combination thereof. Forming housing 12 from plastic also makes housing relatively inexpensive so that it can be disposed of or recycled after a single use. The rigid nature of housing 12 provides stability to bioreactor 10 and enables it to be self-supporting for proper operation during its different modes of operation, as discussed below. However, in alternative embodiments, housing 12 can be formed from a material that will have some bowing, flexing and/or expansion during use but will still be sufficiently rigid to be self-supporting. In still other alternative embodiments, as discussed further below, housing 12 can comprise a collapsible bag made of one or more sheets of polymeric film that is supported within a reusable support housing that is self-supporting, e.g., can be made of the same materials and have the same properties as housing 12, discussed above.

As also shown in FIG. 3, a first transfer opening 40 is formed on and extends through front wall 28/front face 20 so as to communicate with compartment 38. As discussed below, first transfer opening 40 is used to enable the transfer of gas into and out of compartment 38 and, more specifically, into and out of the suspension housed therein, particularly when bioreactor 10 is being used in the static mode. First transfer opening 40 needs to be large enough to facilitate the needed gas transfer, notably CO₂ and oxygen, to the cells within compartment 38 to keep the cells healthy and expanding. In one embodiment, front wall 28/front face 20 has an exterior surface 42 with an area, the area including the area through which first transfer opening 40 passes. In one exemplary embodiment, the area of first transfer opening 40 is typically at least 10%, 20%, 30%, 40%, 50%, 60% 70%, 80%, or 90% of the area of exterior surface 42 or is in a range between any two of the foregoing percentages.

Returning to FIG. 1, housing 12 is elongated having a height longer than a width. Specifically, housing 12 has a height H extending between top end wall 14 and bottom end wall 16 and a width W or diameter. The width W can extend between sidewalls 32 and 34, i.e., a width of front wall 28, or between front wall 28 and back wall 30. The height and width W can vary significantly depend on the selected volume for compartment 38. In some common embodiments, height H is at least or less than 0.2 meters, 0.3 meters, 0.4 meters, 0.6 meters, 0.8 meters, 1 meter or is a range between any two of the foregoing. The maximum or minimum width W or diameter, in some exemplary embodiments, is commonly at least or greater than 2.5 cm, 5 cm, 7.5 cm, 10 cm, 15 cm, 20 cm, 30 cm, 40 cm, 50 cm or in a range between any two of the foregoing. In one embodiment, the maximum height H is at least 1.2, 1.4, 1.6, 1.8, 2, 2.5, 3, 4, of 5 times greater than the maximum width W or diameter or is in a range between any two of the foregoing values. Again, other dimensions can also be used depending on the intended application. As discussed further below, this elongated configuration can also help optimize mixing efficiency and sparging efficiency when in the dynamic mode.

In reference to FIGS. 1 and 3, bioreactor 10 further comprises a gas permeable membrane 50 disposed on housing 12 so as to cover at least a portion of first transfer opening 40. Gas permeable membrane 50 permits the transfer of gases, notably oxygen and CO2, therethrough. Although not required, gas permeable membrane 50 is typically impermeable to liquid so that liquid cannot leak through and, more specifically, is impermeable to media used in the cell suspension. That is, although water vapor may be able to permeate through gas permeable membrane 50, liquid typically cannot. For example, in one exemplary embodiment, when compartment 38 is filled with liquid, such as media or cellular suspension, the liquid will not leak through gas permeable membrane 50 while under ambient conditions, i.e., no external force is being applied to the liquid. As such, gas permeable membrane 50 helps to maintain compartment 38 sterile by preventing contaminates from passing therethrough. In general, gas permeable membrane 50 needs to be sufficiently permeable that based on the oxygen concentration gradient between oxygen within the suspension housed in housing 12/compartment 38 and oxygen in the surrounding environment, oxygen can pass through gas permeable membrane 50 and into the suspension for oxygenating the cells. The same is also true for CO₂ which must be able to pass through gas permeable membrane 50. Thus, in some embodiments gas permeable membrane 50 is a diffusive gas permeable membrane that only permits gas to pass therethrough by passive gas exchange through diffusion. That is, in some embodiments, no pores exist in gas permeable membrane 50 and gas can only pass therethrough on a molecular level by applying a diffusion gradient across gas permeable membrane 50.

In exemplary embodiments, the gas permeability of gas permeable membrane 50 can be in a range between 500 mL/(m²*day) and 25,000 mL/(m²*day) with 5,000 mL/(m²*day) and 10,000 mL/(m²*day) being more preferred. The gas permeability is also typically lower than 75,000 mL/(m²*day), 100,000 mL/(m²*day), 125,000 mL/(m²*day), or 150,000 mL/(m²*day). In one exemplary embodiment, gas permeable membrane 50 comprises a sheet or film of gas permeable silicone, dimethyl silicone, expanded polytetrafluoroethylene (ePTFE), FEP, or fluoropolymer. Examples of other materials and properties for gas permeable membrane 50 are those previously discussed with regard to the definition of “gas permeable membrane.” One specific example of a material that can be purchased and operates as gas permeable membrane 50 is 80 M CUPRAPHAN®. Other materials known in the art can also be used. In some embodiments, the membrane is monolithic material. In some embodiments, the gas permeable membrane may contain inner support structures and material that do not necessarily contribute to gas permeability but do provide physical strength or support structure.

Gas permeable membrane 50 is attached to housing 12 so as to seal first transfer opening 40 closed so that liquid cannot pass therethrough. Gas permeable membrane 50 can be attached to housing 12 in a variety of different ways. For example, gas permeable membrane can be directly secured to housing 12 by welding or adhesive. However, connecting gas permeable membrane 50 directly to housing 12 can often be difficult as a result of incompatibility between materials. As such, in exemplary embodiments, a support frame can be used to connect gas permeable membrane 50 to housing 12.

Specifically, gas permeable membrane 50 is shown as having a outside face 52 and an opposing inside face 54 that each extend to an encircling perimeter edge 56. A support frame 58 has a front face 60, an opposing back face 62 and an interior surface 64 which encircles a passageway 66 that extends between front face 60 and back face 62. Interior surface 64 has an annular recess 68 formed thereon which is configured to receive perimeter edge 56 of gas permeable membrane 50 so as to form a liquid tight seal therebetween. For example, perimeter edge 56 of gas permeable membrane 50 can be sealed within recess 68 by adhesive, press-fit, welding, crimping, or other traditional techniques depending on the material properties of support frame 58 and gas permeable membrane 50. In other embodiments, support frame 58 can comprise two overlapping layers where perimeter edge 56 of gas permeable membrane 50 is sandwiched and secured therebetween. Support frame 58 is typically made of a material compatible for attachment with housing 12. For example, in exemplary embodiments, back face 62 of support frame 58 can be secured to exterior surface 42 of front wall 28, such as through the use of welding, adhesive, fasteners or the like. Welding can have advantages in that it eliminates possible contamination from adhesives and commonly seals better than fasteners. In other embodiments, support frame 58 can be secured directly within first transfer opening 40.

In one embodiment, support frame 58 is made from the same materials as discussed above with regard to housing 12. Where housing 12 comprises a collapsible bag, as previously referenced, gas permeable membrane 50 can be secured to the bag so as to cover an opening passing through a wall of the bag. Gas permeable membrane 50 can be secured to the bag by either first securing gas permeable membrane 50 to a support frame, as discussed above, and then securing the support frame to the collapsible bag or by directly welding or otherwise securing gas permeable membrane 50 to the bag. In some embodiments, the support frame may include interstitial support structures beyond perimeter edge attachment to facilitate support and positioning of the membrane. The support frame can also include a window with inner supports that facilitate support and positioning of the membrane.

It is noted that gas permeable membrane 50 does not function as a sparger. That is, in one exemplary embodiment, the permeability of gas permeable membrane does not permit gas bubbles to pass therethrough. Furthermore, gas permeable membrane 50 is typically in the form of a flat panel or sheet where inside face 54 faces and directly communicates with compartment 38 while outside face 52 faces and directly communicates with an environment outside of compartment 38. In addition, gas permeable membrane 50 is typically void of any internal compartment to which a sparging gas could be delivered and is typically not directly coupled with any tubular member capable of delivering a sparging gas to gas permeable membrane 50. In addition, gas permeable membrane 50 is typically in the form of a single continues panel or sheet as opposed to two or more separate panels or sheets that are overlayed and coupled together.

The present disclosure also includes means for stacking a plurality of bioreactors 10 on top of each other when bioreactors 10 are horizontally or vertically disposed in a static or dynamic mode, so that a gap is formed between the bioreactors. For example, in one embodiment shown in FIGS. 1 and 2, a plurality of first mounts 80 are formed on front wall 28 while a plurality of spaced apart second mounts 82 are formed on back wall 30. Mounts 80 and 82 are configured so that for a plurality of identical bioreactors 10, first mounts 80 of a first bioreactor 10 can engage with second mounts 82 of a second bioreactor 10 so as to form a secure engagement therebetween that provides spacing between the adjacent bioreactors. It is appreciated that mounts 80 and 82 can have a variety of different configurations. In the depicted embodiment, first mounts 80 comprise four separate and spaced apart first mounts 80A-80D that outwardly project front wall 28. In one embodiment, first mounts 80A-80D are disposed at or adjacent to the four corners of front wall 28. Each first mount 80A-80D comprises a body 84 having a recess 86 formed at a terminal end face thereof. Second mounts 82 likewise comprise four mounts 82A-82D outwardly projecting from back wall 30. Second mounts 82A-82D can be disposed at or adjacent to the corners of back wall 30 but, in any event, are positioned so that they align with first mounts 80A-80D of an adjacent bioreactor 10.

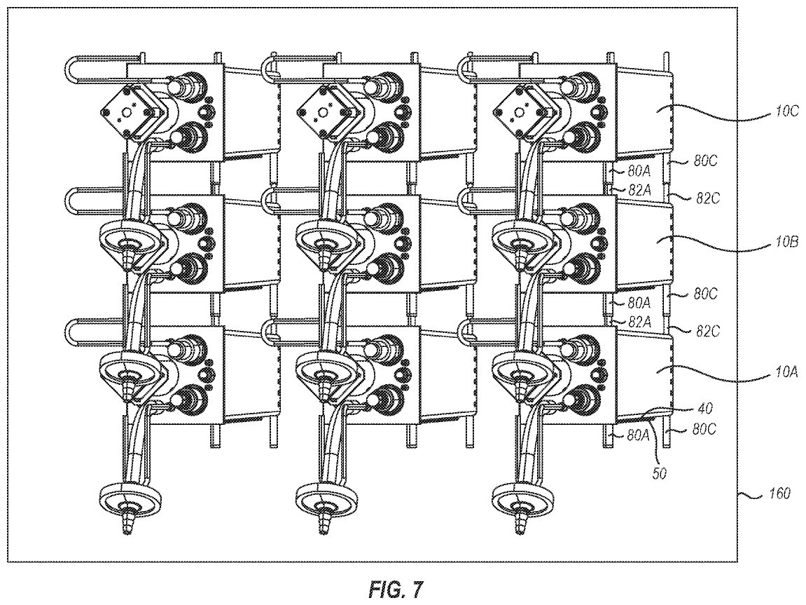

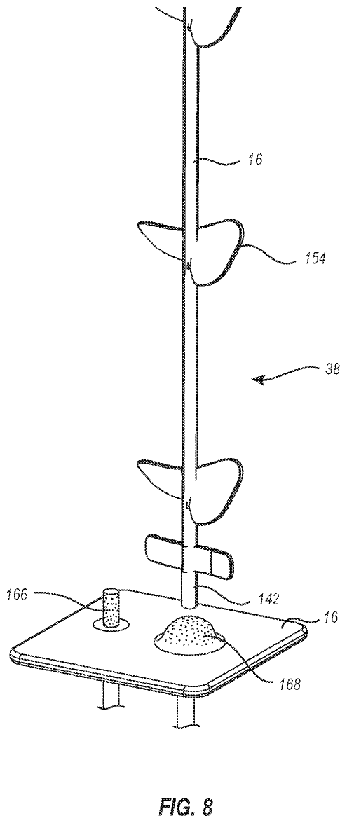

Each second mount 82A-82D terminates at an end 88 that is configured to be received within recess 86 of first mounts 80A-80D. Accordingly, as depicted in FIG. 7, during use, as will be discussed below in greater detail, the plurality of identical bioreactors 10A, 10B, and 10C can be stacked on top each other when in a horizontal orientation by coupling first mounts 80A-80D with second mounts 82A-82D of adjacent bioreactors. In alternative embodiments, first mounts 80A-80D and second mounts 82A-82D can releasably lock together, such as through the use of one or more fasteners or latches, so as to add further stability and prevent unwanted separation.

The coupling between mounts 80 and 82 provides a secure and stable assembly of the plurality of bioreactors and also provides spacing between the bioreactors so that gas can freely flow through gas permeable membrane 50. Mounts 80 also provide a stand for the lowest bioreactor 10 so as to support housing 12 of the lowest bioreactor 10 off of the surface on which mounts 80 are resting. Again, this spacing, as discussed below, enables gas to freely flow through gas permeable membrane 50. It is appreciated that mounts 80 and 82 can have a variety of different configurations. For example, mounts 80 and 82 can be reversed. In other embodiments, mounts outwardly project from one of front wall 28 or back wall 30 while the mounts on the other of the front wall 28 or back wall 30 can comprise recesses that are configured to receive the other mounts. In other embodiments, mounts my only be required of one of the front wall 28 or back wall 30 where the mounts are configured to securely engage with housing 12 of an adjacent barrier without engaging with other mounts. In still other embodiments, it is not required that mounts be directly formed on housing 12. For example, separate and removable racks/mounts can be placed between adjacent bioreactors 10 to facilitate secure stacking and spacing therebetween. In other embodiments, mounts 80 and 82, or the alternatives thereof as discussed above, can also be positioned at top end wall 14 and/or bottom end wall 16 to enable vertical stacking of bioreactors 10 when bioreactors 10 are in a vertical orientation. This can be especially useful when a gas permeable membrane is placed on bottom end wall 16 of bioreactors 10, as discussed below with regard to FIG. 9. Other configurations can also be used.

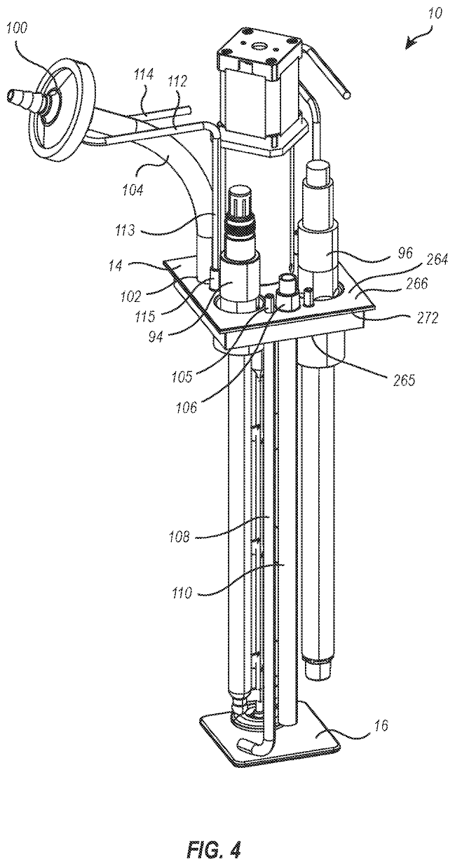

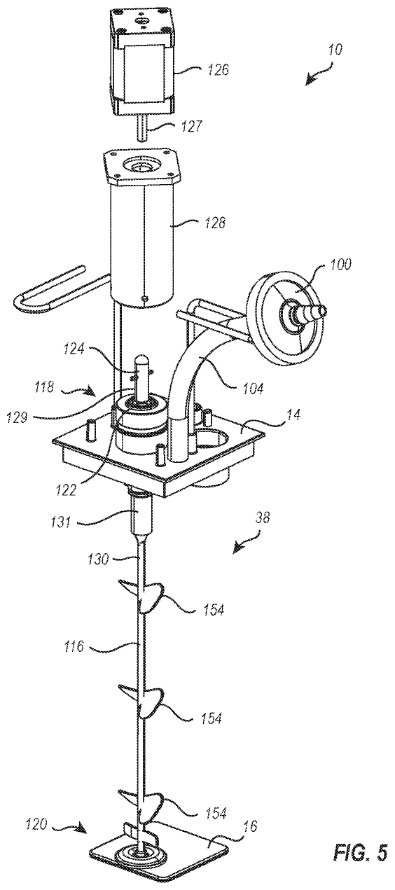

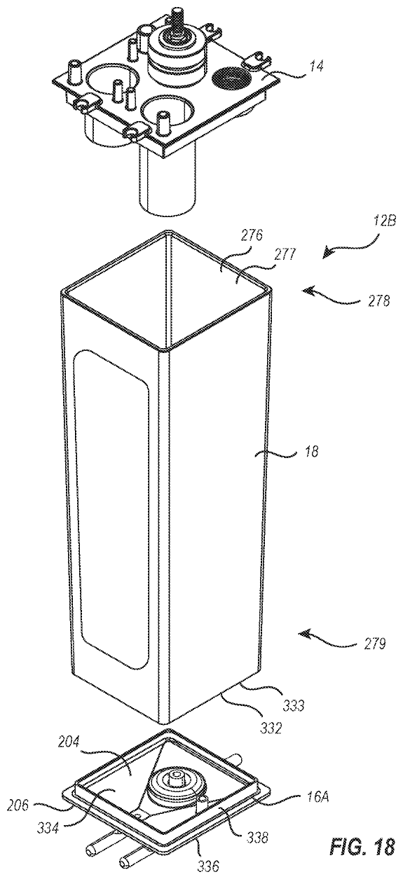

In the depicted embodiment, top end wall 14 is formed separately from encircling sidewall 18 and is configured to be mounted at an upper end thereof. With reference to FIG. 4, top end wall 14 generally comprises an exterior surface 264 and an opposing interior surface 265. Top end wall 14 is mounted at an upper end of encircling sidewall 18 so that interior surface 265 faces compartment 38. More specifically, in one exemplary embodiment, top end wall 14 comprises a top panel 266 having exterior surface 264 and opposing interior surface 265 which each extend to a perimeter edge 272. A mounting lip 274 downwardly projects from interior surface 265 so as to be slightly inset from perimeter edge 272 and forms a continuous loop. With reference to FIGS. 1 and 4, during assembly, mounting lip 274 is slid into an opening 276 formed at an upper end 278 of encircling sidewall 18 while interior surface 265 of top panel 266 sits on an upper edge 277 (see FIG. 18) of encircling sidewall 18. Mounting lip 274 is sealed to the interior surface of encircling sidewall 18 so as to close opening 276. The sealed engagement can be achieved by welding, adhesive, press fit connection, use of a seal ring or other conventional techniques. The above configuration for top end wall 14 simplifies production of both top end wall 14 and encircling sidewall 18 and provides an easy engagement therebetween. It is appreciated, however, that top end wall 14 can also have a variety of other configurations for mounting at upper end 278 of encircling sidewall 18.

Ports 105 and 106 also shown formed on top end wall 14 and communicating with chamber 38. Coupled with ports 105 and 106 are tubes 108 and 110, respectively, that project from top end wall 14 towards bottom end wall 16. Tubes 108 and 110 can be used in conjunction with ports 105 and 106 to delivering fluids and/or components into compartment 38 and/or removing all or some of the suspension from withing compartment 38. During operation, ports 105 and 106 are either closed or are coupled with further tubing.

Turning to FIGS. 4 and 5, bioreactor 10 further comprises a first sensor 94 and a spaced apart second sensor 96. Sensors 94 and 96 are mounted on top end wall 14 and project down into compartment 38 towards bottom end wall 16. Sensors 94 and 96 can each comprise a sensor common to other conventional bioreactors, such as a pressure sensor, temperature sensor, foam sensor, glucose sensor, pH sensor, DO sensor, CO₂ sensor, density sensor, cell density sensor or the like. Although two sensors 94 and 96 are shown, in alternative embodiments, bioreactor 10 can have at least 2, 3, 4, or more sensors mounted on housing 12, such as top end wall 14, and projecting in compartment 38. For example, bioreactor 10 will commonly include a temperature sensor, pH sensor, DO sensor, and a CO₂ sensor. Other sensors can also be added. As will be discussed below in more detail, it is appreciated that the sensors can have a variety of different configurations and can be placed in a variety of different locations on housing 12.

Bioreactor 10 also includes a gas filter 100 that communicates with compartment 38. Specifically, in one exemplary embodiment, a port 102 is formed on top end wall 14 that communicates with compartment 38. A tube 104 couples with port 102 and extends to gas filter 100. As will be discussed below in greater detail, during operation in the dynamic mode, gas is sparged into compartment 38 and passes through the suspension. Gas filter 100 acts as a one-way valve that enables the gas to exit compartment 38 while also preventing containments from passing into compartment 38. Specifically, in one embodiment, gas filter 100 is a sterilizing filter having a pore size small enough to prevent contaminants from passing therethrough but allowing the gas to escape therethrough. In the depicted embodiment, a hanger 112 is optionally mounted to housing 12/top end wall 14 and is used to support tube 104/gas filter 100, thereby stabilizing gas filter 100 and preventing kinking of tube 104. Hanger 112 comprises a primary rod 113 vertically upstanding from top end wall 14 and a U-shaped support rod 114 radially outwardly projecting from the free end of primary rod 113. Tube 104 is received within the slot bounded by support rod 114. Hanger 112 is removably received within a tubular stand 115 upwardly projecting from top end wall 14. Other forms of hangers can also be used or, alternatively, hanger 112 can be eliminated.

As shown in FIG. 1, a plurality of optional ports 92 can also be formed on encircling sidewall 18 that communicate with compartment 38. For example, ports 92 can be formed on sidewall 32 and/or 34 adjacent to or toward bottom end wall 16. Ports 92 can be used for coupling with tubes for passing fluids or other components into and/or out of compartment 38 and/or can be used for receiving different types of sensors, such as those previously discussed above with sensors 94 and 96. Depending on the orientation and mode of operation of bioreactor 10, it can preferred to have ports 92 at other locations than on top end wall 14 so that the related tube(s) and/or sensors can more efficiently communicate with the suspension within compartment 38 depending on the orientation of bioreactor 10. Ports 92 can also be placed on any of the other walls of encircling sidewall 18.

Continuing with FIG. 5, bioreactor 10 also includes a mixing element that is movably disposed within compartment 38 for mechanically mixing the suspension within compartment 38 when bioreactor is in the dynamic mode. Specifically, as shown in FIG. 5, a drive shaft 116 is provided extending between a first end 118 and opposing second end 120. Drive shaft 116 projects from top end wall 14, into compartment 38 and towards bottom end wall 16. More specifically, in one exemplary embodiment, first end 118 of drive shaft 116 passes through top end wall 14 and is rotatably coupled thereto by a dynamic seal 122. Dynamic seal 122 enables drive shaft 116 to rotate therein while preventing outside contaminates from passing into compartment 38. A terminal end 124 of first end 118 projects outside of dynamic seal 122/top end wall 14. First end 118 of drive shaft 116 and, more particularly, terminal end 124, couples with a drive motor 126 that is typically electric. Drive motor 126 can removably mounted on housing 12 and, more particularly, to top end wall 14, such as through an optional tubular guard sleeve 128 that encircles first end 118 of drive shaft 116. During use, a drive shaft 127 of drive motor 126 can removably couple with terminal end 124 of drive shaft 116 such that activation of drive motor 126 facilitates rotation of drive shaft 116. When drive shaft 116 is not in use, drive motor 126 and/or guard sleeve 128 can be removed from housing 12 so as to minimize the size of bioreactor 10. For ease in production, in one exemplary embodiment drive shaft 116 can comprise an upper section 129 which passes through dynamic seal 122 and a lower section 130 disposed within compartment 38. Upper section 129 and lower section 130 can be removably or permanently coupled together by a coupling 131 located within compartment 38.

In alternative exemplary embodiments, drive shaft 116 can be modified to rotate through magnetic coupling. For example, a lower section 130 of drive shaft 116 can be sealed within compartment 38 and have a magnetic coupler at one end. A magnetic driver can be disposed outside of housing 12 and magnetically coupled with the magnetic coupler. In turn, as is known in the art, activation of the magnetic driver outside of housing 12 facilitates rotation of drive shaft 116/lower section 130 within compartment 38. In this embodiment, dynamic seal 122 can be eliminated and there is lower risk of leaking or contamination.

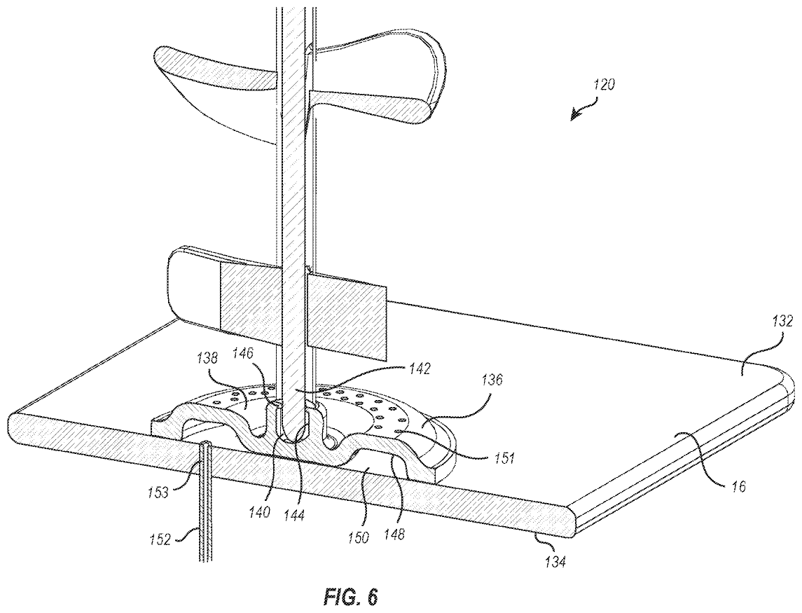

Second end 120 of drive shaft 116 extends towards bottom end wall 16 and, in the depicted embodiment, can be rotatably supported on bottom end wall 16. For example, turning to FIG. 6, bottom end wall 16 has an interior surface 132 and an opposing exterior surface 134. Mounted on interior surface 132 is an optional steady support 136. Steady support 136 has a top surface 138 with a recess 140 formed thereon. Second end 120 of drive shaft 116 terminates at a terminal end 142 that is rotatably disposed within recess 140. More specifically, in one exemplary embodiment, steady support 136 has a boundary face 144 that bounds recess 140. An annular lip seal 146 radially inwardly projects from boundary face 144 so as to encircle and form a liquid tight seal against terminal end 142 of drive shaft 116. That is, lip seal 146 seals against drive shaft 116 so as to prevent the suspension within compartment 38, or at least the cells therein, from passing into and stagnating within recess 140. However, steady support 136 still permits drive shaft 116 to rotate relative to steady support 136. Boundary face 144 can be in the form of a curved pocket on which terminal end 142 of drive shaft 116 is directly supported. Terminal end 142 can also terminate in a rounded tip to help facilitate smooth rotation of drive shaft 116 on boundary face 144.

In exemplary embodiments, a sparger can be incorporated into steady support 136 for delivering gas bubbles into compartment 38. For example, steady support 136 also has an interior surface 148 that at least partially bounds a cavity 150. In one embodiment, steady support 136 can be welded or otherwise secured to interior surface 132 of bottom end wall 16 so that cavity 150 is bounded between interior surface 132 of bottom end wall 16 and an interior surface 148 of steady support 136. Alternatively, steady support 136 can also be formed with a bottom wall that extends across the base of steady support 136 so that cavity 150 is bounded between interior surface 132 and the bottom wall. A plurality of spaced apart gas openings 151 extends between top surface 138 and interior surface 148 of steady support 136. A gas line 152 couples with a port 153 on bottom end wall 16 so as to communicate with cavity 150. Gas line 152 is coupled with a gas source. During operation, gas is delivered from the gas source, through gas line 152, and into cavity 150. The gas then travels out through gas openings 151 so as to enter the suspension within compartment 38 in the form of bubbles. The gas bubbles flow up through the suspension and the gas subsequently exits compartment 38 through gas filter 100 (see FIG. 5), as previously discussed. In alternative embodiments, as discussed below, other forms of gas spargers can also be used. For example, the sparger need not be incorporated into steady support. Rather, the sparger can be separate and discrete from steady support 136.

Returning to FIG. 5, mounted on drive shaft 116 are a plurality of spaced apart mixing elements 154. In the depicted embodiment, three separate mixing elements 154 are shown disposed on drive shaft 116. In alternative embodiments, it is appreciated that drive shaft 116 can be provided with at least one, two, three, four or more separate mixing elements 154. Mixing elements 154 can comprise an impeller, paddle, fin, projection, or other structure that when moved by drive shaft 116/drive motor 126 facilitate mixing of the suspension within compartment 38.

During operation, bioreactor 10 can be initially disposed in a horizontal orientation and operated in the static mode, with no mixing, for a first period of time. Once the cells achieve a desired density, state or satisfy some other predetermined condition, bioreactor 10 can be rotated to a vertical orientation and operated in the dynamic mode wherein mixing occurs for a second period of time, a first second phase of cell growth, or other phase of cell processing. In an alternative exemplary embodiment, bioreactor 10 can also be operated in the dynamic mode, with rotation of the drive shaft 116 and mixing elements 154, while bioreactor 10 is in the horizontal orientation and during a first period of time, a first phase of cell growth, or other phase of cell processing.

More specifically, during use in one exemplary embodiment, the suspension is disposed within compartment 38 by dispensing cells, media, nutrients and other desired components into compartment 38, such as through port 105 and/or 106. All or some of the components can be combined outside of compartment 38 and then dispensed therein or the components can be separately dispensed into compartment 38 so as to form the suspension therein. As shown in FIG. 7, either prior to or after forming suspension within compartment 38, bioreactor 10 (shown as bioreactor 10A) is positioned within a temperature and pressure-controlled environment, such as within an incubator 160. Bioreactor 10 is placed in a horizontal orientation so that first transfer opening 40/gas permeable membrane 50 (see FIG. 3) is facing downward. More specifically, as depicted in FIG. 3, compartment 38 has a central longitudinal axis 162 that centrally extends through compartment 38 so as to pass through top end wall 14 and bottom end wall 16. When in the horizontal orientation, bioreactor 10 is disposed so that front wall 28 is now the bottom surface and axis 162 is oriented so as to be at any angle in a range between +/−30 degrees, 20 degrees, 15 degrees, 10 degrees, or 5 degrees. The “any angle” also includes axis 162 being horizontally disposed. That is, when the horizonal orientation is used for operation of bioreactor 10 in the static or dynamic mode, axis 162 is commonly disposed horizontally. However, bioreactor 10 can also operate in the static or dynamic mode if axis 162 is angled slightly relative to horizontal. Bioreactor 10 can also be operated in static mode, with no mixing or no rotation/actuation of drive shaft 116/mixing element 154, while bioreactor 10 in the vertical orientation and is equipped with a gas permeable membrane positioned at bottom end wall 16 (as in FIG. 9). Likewise, static mode operation can be utilized while in the vertical orientation in conjunction with forced gas exchange through gas permeable membrane 172 and gas phase of the bioreactor contents.

Placing bioreactor 10 in the horizontal orientation with first transfer opening 40/gas permeable membrane 50 facing downward for operation during the static mode helps to maximize the amount of cell suspension contacting gas permeable membrane 50 so that the gas content within the suspension is more accurately controlled. In addition, with first transfer opening 40/gas permeable membrane 50 facing downward, the cells within the suspension settle under the force of gravity so as to rest directly against or be disposed adjacent to gas permeable membrane 50, thereby helping to ensure proper gas transfer to the cells. Furthermore, because mixing element 154 and the sparger are typically not operated during static mode when the bioreactor includes a gas permeable membrane 50, no consideration needs to be made with regard to optimal mixing efficiency or optimal sparging efficiency during static mode operation.

As previously discussed and depicted in FIG. 7, a plurality of bioreactors 10A-10C can be simultaneously positioned within incubator 160 (or other temperature and pressure-controlled environment) to optimize cell production. To optimize the use of space within incubator 160, select incubators, or a plurality of incubators, bioreactors 10A-10C can be vertically stacked on top of each other while in the horizontal orientation by using mounts 80 and 82, as previously discussed. For example, bioreactors 10A-10C are shown stacked within incubator 160 with each bioreactor 10A-10C having the same configuration. Each of the plurality of bioreactors 10A-10C have the same or substantially the same horizontal orientation and are vertically spaced apart from each other to enable the free flow of gas through gas permeable membrane 50. Depending on the size of bioreactors 10 and the size of incubator 160, bioreactors can be stacked at least 2, 3, 4, 5, 6 or 8 high while at least 2, 4, 8, 12, 16 or 24 bioreactors 10 can be housed within incubator 160 (or other temperature and pressure-controlled environment) in the horizontal orientation. As previously discussed, bioreactors 10 can also be vertically stacked within incubator 160 or other temperature and pressure-controlled environment while in vertical orientations. Based on the various sensor readings, the temperature and gas compositions, concentrations, and/or pressures within each bioreactor 10 can be selectively controlled by adjusting the temperature and gas composition, concentration, and/or pressure within incubator 160 (or other temperature and pressure-controlled environment). For example, as previously mentioned, by controlling the oxygen concentration gradient between the suspension and the environment within incubator 160, oxygen can be passed through gas permeable membrane 50 to cells within the suspension through gas diffusion. Likewise, controlling the CO₂ concentration gradient between the suspension and the environment with incubator 160 can be used to help control the pH of the suspension.

During the expansion of cells, while bioreactor 10 is in the static mode, there is typically no mixing or disturbing of the cell suspension. For example, drive motor 176 is typically not activated to facilitate movement of mixing elements 154. Furthermore, there is typically no movement of bioreactor 10 such as by tilting, rocking, or swiveling and no disturbing of the cells therein by the injection of liquid or gas into compartment 38. In addition, because of the gas transfer through gas permeable membrane 50, no sparging of gas into compartment 38 is typically required. In some embodiments, gas flow through the gas phase and not the liquid of the bioreactor contents can be performed to facilitate gas exchange without perturbing the liquid. The cells are expanded in bioreactor 10 in the static mode for a first period of time which is typically at least 3 hours, 6 hours, 12 hours, 18 hours, 24 hours, 36 hours, 48 hours, 72 hours, 120 hours or in a range between any two of the foregoing. However, in some situations, it may be beneficial to the growth of the cells to facilitate some intermittent perturbing, i.e., momentary movement, of the cells with the bioreactor 10 operating in dynamic mode by activating, actuating or rotating the drive motor 176, drive shaft 116 and/or mixing element 154 or by shaking, rocking, angling or otherwise perturbing the bioreactors 10. The perturbing of the cells may be achieved by a short duration of tilting, rocking, or swiveling of bioreactor 10, movement of mixing element 154 within bioreactor 10, injection of gas or liquid into compartment 38 or other techniques while bioreactor 10 is operated in the dynamic mode within incubator 160. For example, the perturbing of the cells, such as by using one of the above processes, may occur for a perturbing period of less than 15, 10, 5, 3, 2, 1, 0.5 or 0.1 minutes followed by a stagnant period of at least 0.5, 1, 12, 24 or 48 hours where no force is applied to disturb the suspension/cells, e.g., the bioreactor is operated in static mode. The perturbing period and stagnant period can be repeated multiple times or continuously while bioreactor 10 is operated in alternating static mode and dynamic modes within incubator 160 in the horizontal orientation (shown in FIG. 7), in the vertical orientation (shown in FIG. 9), or other angled orientation.

Subjecting the cells/suspension to static mode where there is typically no mixing or disturbing of the cell suspension for a first period of time may be used to help facilitate treatment or post treatment recovery of the cells. For example, in some applications, it may be desired to treat the cells with a lentivirus. Lentiviruses can integrate a significant amount of viral complementary DNA into the DNA of the host cell and can efficiently infect nondividing cells, so they are one of the most efficient methods of gene delivery. In some cases, transduction, transfection, application, integration or infiltration of the lentivirus into a cell may be more effective if completed in static mode, where no mixing or perturbation occurs.

In other applications, either prior to or after placing the suspension within compartment 38 of bioreactor 10, the cells/suspension may be subject to electroporation. Electroporation is a microbiology technique in which an electrical field is applied to the cells in order to increase the permeability of the cell membrane, allowing chemical, drugs, electrode arrays, or DNA to be introduced into the cell. Electroporation disrupts cell membranes and they often need a static recovery directly thereafter with proper O₂ and CO₂ to maintain viability. Significant stirring of the cells directly after electroporation can result in a loss of viability. As such, recovery of the cells may be improved if initially treated under static mode after electroporation.

Once a predetermined time period has passed or it is detected that the cells have reached a predefined state or density or have satisfied some other predefined condition, bioreactor 10 can operated in a dynamic mode or a constant dynamic mode. For example, once the cells reach a predefined density, it is necessary to add additional media into compartment 38 to enable continued expansion of the cells. However, as the volume of suspension increases within compartment 38, the volume of suspension eventually reaches a point where gas transfer solely through gas permeable membrane 50 is inadequate to maintain viability of the cells. When bioreactor 10 is operated in dynamic mode, gas can be sparged into compartment 38 while the cells are being mixed therein, thereby increasing gas transfer to the cells. The bioreactor can be placed in a vertical or horizontal position when operating in dynamic mode.

In an exemplary embodiment, bioreactor 10 is in the vertical orientation when in dynamic mode used to accelerate cell growth after cell density reaches a predetermine threshold. To move to a vertical orientation, as depicted in FIG. 3, a horizontal bioreactor 10 is moved approximately 90 degrees so that bottom end wall 16 is now the bottom surface and first transfer opening 40/gas permeable membrane 50 is projecting laterally. More specifically, bioreactor 10 can be oriented so that central axis 162 is oriented at any angle in a range between +/−30 degrees, 20 degrees, 15 degrees, 10 degrees, or 5 degrees relative to vertical. The “any angle” also includes axis 162 being vertically disposed. That is, when in the vertical orientation for operation in the dynamic mode (or static mode), axis 162 is commonly disposed vertically. However, bioreactor 10 can also operate in the dynamic mode if axis 162 is angled slightly relative to the vertical. It is also appreciated that bioreactor 10 can be operated in the static mode when moved to the vertical orientation, as described above, and can be periodically moved between static mode and dynamic mode while in both the horizontal orientation and the vertical orientation.

Once bioreactor 10 is operated in dynamic mode, mechanical mixing of the suspension can be initiated by activating drive motor 126 to rotate drive shaft 116 and thereby move mixing elements 154 within the suspension. In other exemplary embodiments, other types of mixing elements can be used and other mixing techniques, such as tilting, rocking, or swiveling of bioreactor 10 or injecting a gas or liquid into compartment 38, can be used to mix the suspension. Furthermore, gas can be sparged during the dynamic mode into the lower end of compartment 38, such as through steady support 136 or a separate sparger. Additional media and/or nutrients are typically added to compartment 38 either before, during, or after bioreactor 10 is operated in dynamic mode (in vertical or horizontal orientation).

Bioreactor 10 is specifically configured with width W being smaller than height H, as previously discussed with regard to FIG. 1, so as to help optimize performance in both the static mode and the dynamic mode and horizontal and vertical orientations. For example, having a large height H relative to width W while in the horizontal orientation, enables maximizing the surface area of gas permeable membrane 50 in contact with the suspension while minimizing the height of suspension above gas permeable membrane 50, thereby optimizing gas transfer through gas permeable membrane 50 to the cells. In this way, gas exchange and cell expansion can be maximized or optimized while the reactor is in the static mode and horizontal orientation. In turn, having a large height H relative to width W while in the vertical orientation and dynamic mode retains the suspension closer to drive shaft 116/mixing elements 154, which helps to optimize both mechanical mixing efficiency and sparging efficiency within compartment 38 at various levels of liquid volume.

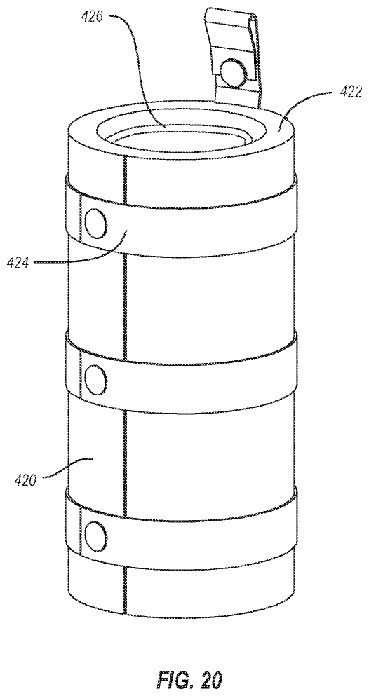

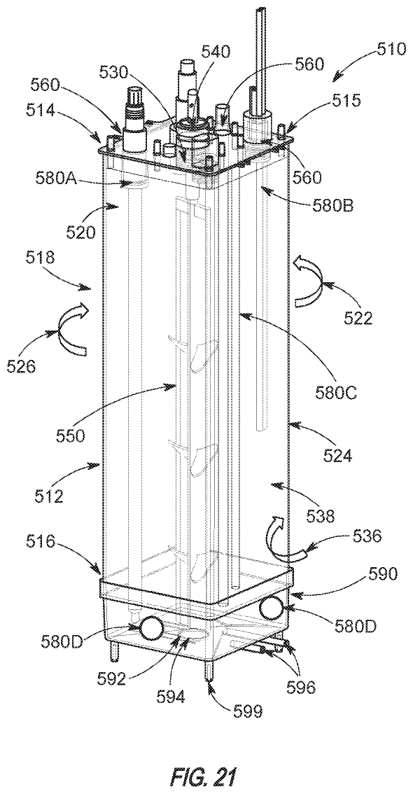

Bioreactor 10 can be retained within incubator 160 (or other temperature and pressure-controlled environment) when operated in dynamic mode in both the vertical and horizontal orientations to help control temperature and other environmental conditions. More commonly, however, bioreactor 10 is moved outside of incubator 160 when operated in dynamic mode and the operating conditions separately controlled. For example, with reference to FIGS. 1 and 20, a heating jacket 420 can be disposed around housing 12 to control the temperature of the suspension within compartment 38 when bioreactor 10 is operated in dynamic mode outside of incubator 160. In one exemplary embodiment, heating jacket 420 can include an insulation pad 422 that can be wrapped in a loop around housing 12 and held in the desired configuration by straps 424 that encircle the exterior of pad 422. Disposed either within pad 422, or on an interior surface thereof, is electrical heating tape 426 or other heating elements for controlling the temperature of the suspension within compartment 38. As discussed below, other types of heating jackets or heating mechanisms can also be used for controlling the temperature of the suspension within compartment 38. Bioreactor 10 combines with heating jacket 420 to form a bioreactor system.

With bioreactor 10 in the dynamic mode, cells are expanded in compartment 38 for a second period of time, which is typically at least 1 day, 2 days, 7 days, 14 days, 21 days, 60 days or in a range between any two of the foregoing. In one exemplary embodiment, the above sparging and mixing can be continuously operated during the second period of time with bioreactor 10 in the vertical orientation. Once a predetermined time period has passed or it is detected that the cells within compartment 38 have reached a predefined state or density or have satisfied some other predefined condition, the suspension, media, and/or cells can be transferred out of compartment 38 for subsequent use or further processing, such as further cultivation in a larger bioreactor.

Once the suspension or desired fraction thereof has been removed from compartment 38, housing 12 and other components thereof that came in contact with the suspension can be discarded as a single use item. In contrast, elements such as drive motor 126, guard sleeve 128, and parts of the sensors that do not directly contact the suspension can be removed and reused without any required sterilization. In exemplary methods of use, in the static mode and/or dynamic mode, bioreactor 10 can be operated in batch processing wherein the suspension is retained within compartment 38 during processing. In an alternative embodiment, in the static mode and/or dynamic mode, tubing can be coupled to bioreactor 10 outside of compartment 38 to form a circulating loop so that bioreactor 10 can be operated using a perfusion process. When bioreactor 10 is being used with or in a perfusion device, process or mode, cell expansion can occur within bioreactor 10 for an extended period of time, for example, at least 2 days, 5 days, 14 days, 21 days, 30 days or 60 days.

In the above-described process, bioreactor 10 is used sequentially in both the static mode and the dynamic mode. In some cases, however, bioreactor 10 may only be used in one of static mode or dynamic mode and/or in alternating vertical and horizontal orientations in either mode. That is, the cells may be removed from bioreactor 10 after bioreactor 10 is only operated in the static or dynamic mode where bioreactor 10 can be oriented in the horizontal orientation or the vertical orientation in either mode. Furthermore, while bioreactor 10 is operated in the vertical or horizontal orientation and in dynamic mode, bioreactor 10 can be disposed inside of or outside of an incubator (or other temperature and pressure-controlled environment) or can be sequentially moved therebetween. Sparging is typically implemented while operating bioreactor 10 outside of an incubator. However, depending on the conditions within the incubator, sparging may or may not be required while operating bioreactor 10 in the dynamic mode within the incubator.

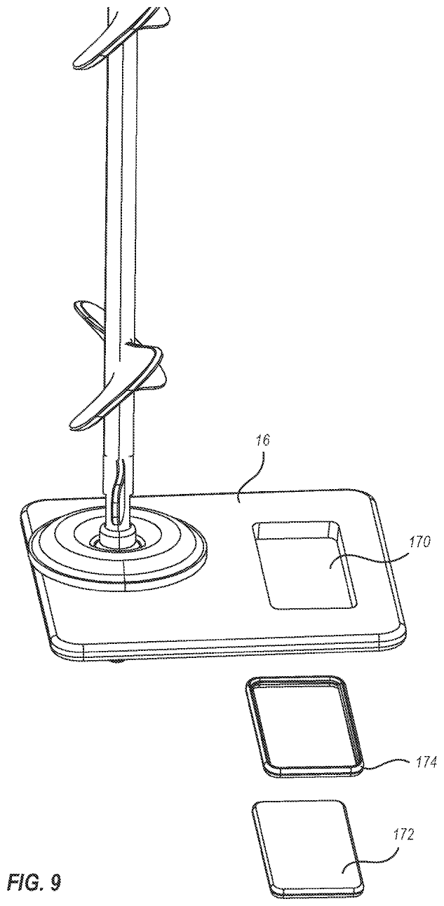

It is appreciated that bioreactor 10 can have a variety of alternative components which can be mixed and matched with other components and embodiments. For example, as depicted in FIG. 8, depending on factors such as the size of housing 12, the amount of suspension being processed, and the configuration of the mixing elements, in some embodiments steady support 136 can be eliminated. In this embodiment, terminal end 142 of drive shaft 116 can be freely suspended within compartment 38 at a distance spaced apart from bottom end wall 16. Furthermore, one or more of a plurality of different types of spargers can be disposed on bottom end wall 16 for sparging gas into compartment 38. For example, the sparger can comprise a porous frit sparger 166 or a dome sparger 168 which both mount on bottom end wall 16 and project into compartment 38. Alternatively, the sparger can comprise a film sparger. The film spargers are flush with the end wall 16 so they do not obstruct the flow of fluid or the flow of cells or microorganisms within the compartment 38. Examples of various types of dome spargers and film spargers that can be used in the present disclosure are disclosed in U.S. Pat. Nos. 8,603,805, 9,005,971, 9,259,692, 9,475,012, 9,682,353, 10,328,404 and 9,643,133, 10,350,554, 10,843,141, and U.S. Publication No. 2021/0069654, which are incorporated herein by specific reference. Other conventional spargers can also be used. In another alternative embodiment, one or more of separate spargers 166 and/or 168 can have steady support 136, as shown in FIG. 6, incorporated therein or can be used independent of steady support 136.

Turning to FIG. 9, in another exemplary embodiment, bottom end wall 16 can be formed with a second transfer opening 170 extending therethrough which is at least partially covered by a gas permeable membrane 172. Gas permeable membrane 172 can have the same properties and composition as previously discussed with regard to gas permeable membrane 50. A support frame 174 having the same configuration, properties and alternatives as previously discussed with support frame 58 can optionally be used to secure gas permeable membrane 172 within second transfer opening 170 or to the exterior surface of bottom end wall 16 for sealing second transfer opening 170 closed while still allowing gas to pass through gas permeable membrane 172. Alternatively, gas permeable membrane 172 can be secured directly to bottom end wall 16 without the use of support frame 174. In other alternative embodiments, a second transfer opening can also be formed extending through sidewall 32 and/or sidewall 34. These openings can likewise be covered by a corresponding gas permeable membrane with or without the use a support frame so as to seal the transfer openings closed while still allowing gas to pass through the gas permeable membranes. These additional transfer openings with gas permeable membranes can be used to supplement gas permeable membrane 50 in transferring gas to the suspension. In some embodiments, only bottom end wall 16 has a gas permeable membrane mounted thereon while front wall 86 has no gas permeable membrane and no transfer opening and is made of entirely the same material as sidewalls 32, 34. Bioreactor 10 shown in the alternative exemplary embodiments of FIGS. 1, 8, and 9, including housing 12, drive shaft 116 and mixing elements 154 thereof, can each be used in dynamic and static mode, in both horizontal and vertical orientations, and both inside and outside an incubator or other temperature and pressure-controlled environment.

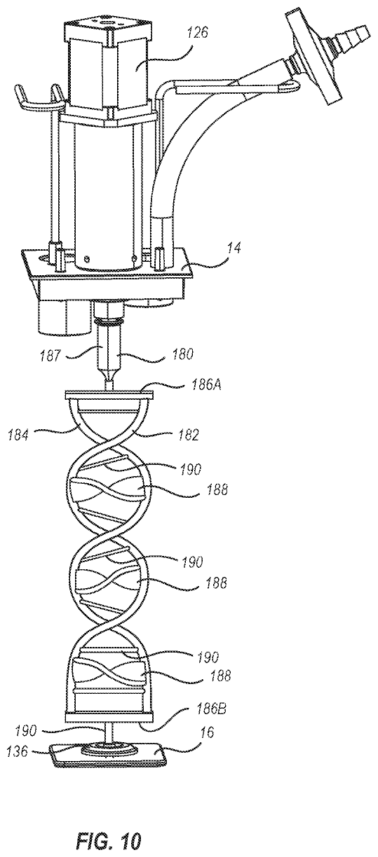

Drive shaft 116 and mixing elements 154 can have a variety of different configurations. For example, FIG. 10 shows a drive shaft 180 coupled with drive motor 126. Drive shaft 180 includes a first drive shaft portion 182 having a helical configuration and a second drive shaft portion 184 having a helical configuration. Second drive shaft portion 184 is disposed laterally to first drive shaft portion 182 and extends along the length of first drive shaft portion 182. Drive shaft portions 182 and 184 are spaced apart along their length and intertwin so as to form a DNA helix configuration. The first ends of drive shaft portions 182 and 184 are connected together by a support 186A while the second ends of drive shaft portions 182 and 184 are connected together by a support 186B. An upper shaft portion 187 projects from support 186A, passes through top end wall 14 and couples with drive motor 126. Support 186B can be freely suspended or can have a lower shaft portion 190 project therefrom and connect to steady support 136. Drive shaft portions 182 and 184 are sufficiently rigid that if twisted under torsion about a longitudinal axis thereof over an angle of at least 45 degrees, 90 degrees, or 180 degrees, they will deform with plastic deformation. Mixing elements 188 extend between drive shaft portions 182 and 184 in the form of impellers. Mixing elements 188 can also have other configurations that will facilitate mixing of the cell suspension when rotated by drive shaft 180. Optionally, spaced apart braces 192 can also extend between drive shaft portions 182 and 184 to help maintain stability therebetween.

Designing drive shaft 180 to include drive shaft portions 182 and 184 having a helical configuration helps to match mixing properties between bioreactor 10 and larger scaled bioreactors that use adjacent drive lines that twist into a helical configuration during the mixing process. Examples of such larger scaled bioreactors are disclosed in U.S. Pat. No. 10,669,515. However, in contrast to the larger bioreactors where the drive lines are flexible lines, drive shaft portions 182 and 184 are rigid members. Thus, although not required, the use of drive shaft 180 can help to maintain uniform mixing of the cell suspension as the cell suspension is moved to larger sized bioreactors. However, in alternative exemplary embodiments, drive portions 182 and 184 can be formed from flexible line. That is, bioreactor 10 and the other bioreactors disclosed herein can be modified to incorporate the mixing assemblies as disclosed U.S. Pat. No. 10,669,515, issued Jun. 2, 2020, which is incorporated herein by specific reference. Bioreactor 10 incorporating drive shaft 180 and mixing elements 188 can also be used in dynamic and static mode, in both horizontal and vertical orientations, and both inside and outside an incubator or other temperature and pressure-controlled environment.

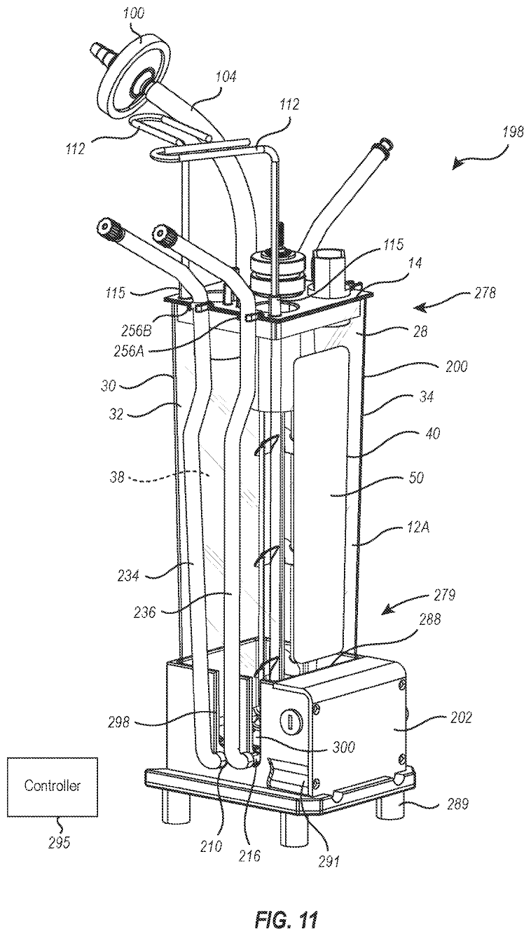

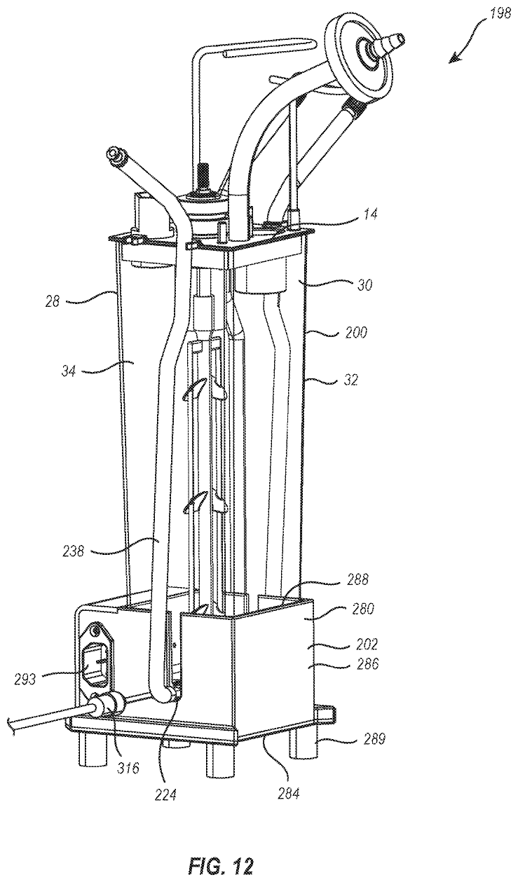

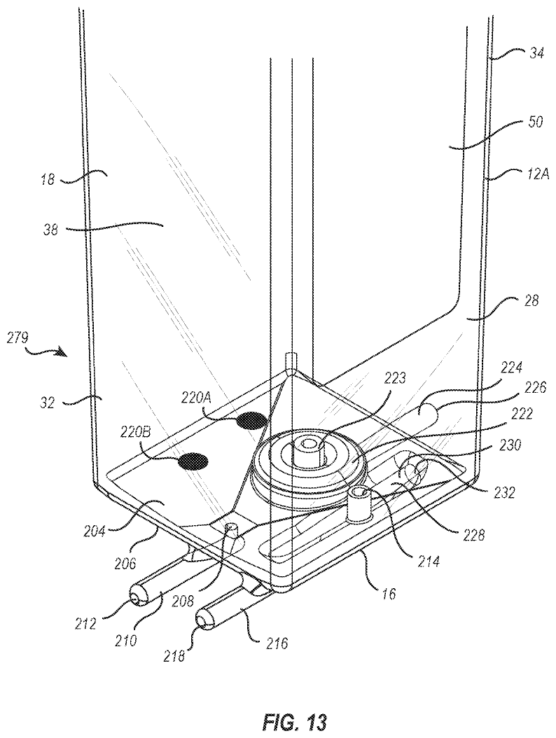

Depicted in FIGS. 11 and 12 is another exemplary embodiment of a bioreactor system 198 incorporating features of the present disclosure. In general, bioreactor system 198 comprises bioreactor 200 removably seated on a heater stand 202. Like elements between bioreactor 200 and bioreactor 10 are identified by like reference numbers. Bioreactor 200 includes a housing 12A having the same walls, faces, surfaces and compartment as housing 12 and thus like elements between housing 12 and 12A are identified by like reference characters. In addition, the disclosure, alternatives, and uses previously discussed with regard to housing 12 are also applicable to housing 12A. For example, housing 12A includes front wall 28, back wall 30 and sidewalls 32 and 34 that each extend between and upper end 278 and an opposing lower end 279. Upper end 278 terminates at top end wall 14 while lower end 279 terminates at bottom end wall 16 (see FIG. 13). All walls except top end wall 14 are shown in this embodiment as being transparent or translucent but could also be formed as opaque. Housing 12A bounds compartment 38. Extending through front wall 28 so as to communicate with compartment 38 is first transfer opening 40. Gas permeable membrane 50 is secured to front wall 28 with or without the use of support frame 58 (see FIG. 3) so as to cover and seal first transfer opening 40. Again, all of the prior disclosure of gas permeable membrane 50 and the mounting on front wall 28 in relation to housing 12 is also applicable to housing 12A.

Turning to FIG. 13, bottom end wall 16 of housing 12A has an interior surface 204 and an opposing exterior surface 206. In the depicted embodiment, bottom end wall 16 is shown being integrally formed with encircling sidewall 18. For example, bottom end wall 16 and encircling sidewall 18 can be molded as one unitary piece. However, in an alternative embodiment, as discussed below, bottom end wall 16 can be formed separately from encircling sidewall 18 and the two elements subsequently connected together. As will also be discussed below in greater detail, a pair of spot sensors 220A and 220B are shown secured on interior surface 204 so as to face exterior surface 206. Interior surface 204 slopes inward and down to a drain port 208. A tubular first stem 210 is disposed on exterior surface 206 of bottom end wall 16 and projects laterally so as to extend past sidewall 32. First stem 210 bounds a channel 212 that communicates with drain port 208. An inlet port 214 is also formed on interior surface 204 and communicates with compartment 38. A tubular second stem 216 is disposed on exterior surface 206 of bottom end wall 16 and projects laterally so as to also extend past sidewall 32. Second stem 216 bounds a channel 218 that communicates with inlet port 214. Stems 210 and 216 can project in parallel alignment.

Also disposed on interior surface 204 of bottom end wall 16 is a sparger 222. Although not required, in this embodiment sparger 222 is donut shaped and encircles a guide stem 223 upwardly projecting from interior surface 204. A tubular third stem 224 is disposed on exterior surface 206 of bottom end wall 16 and projects laterally opposite of stems 210 and 216 so as to also extend past sidewall 34. Third stem 224 bounds a channel 226 that communicates with sparger 222. As such, gas passed through third stem 224 will pass through sparger 222 and enter chamber 38 in the form of bubbles. A tubular fourth stem 228 is disposed on exterior surface 206 of bottom end wall 16 and extends along a length bottom wall 16. Fourth stem 228 bounds a channel 230 that does not communicate with compartment 38 but does communicate with the environment through an opening 232. As will be discussed below in more detail, channel 230 is configured to receive a temperature sensor and can project parallel to third stem 224. In the depicted embodiment, stems 210, 216, 224 and 228 are integrally formed with bottom end wall 16 so as to form a single unitary piece with bottom end wall 16 as opposed to being separately connected thereto. For example, stems 210, 216, 224 and 228 can be integrally molded with bottom end wall 16. In an alternative embodiment, one or more of stems 210, 216, 224 and 228 can be separately connected to bottom end wall 16.

As depicted in FIGS. 11 and 12, a tube 234 couples with first stem 210 and can be used for draining, sampling or otherwise removing all or part of the cell suspension from compartment 38. A tube 236 couples with second stem 216 and can be used for delivering media, nutrients, cell suspension and/or a component thereof into compartment 38. A tube 238 couples with third stem 224 and can be used for delivering a gas to sparger 222.

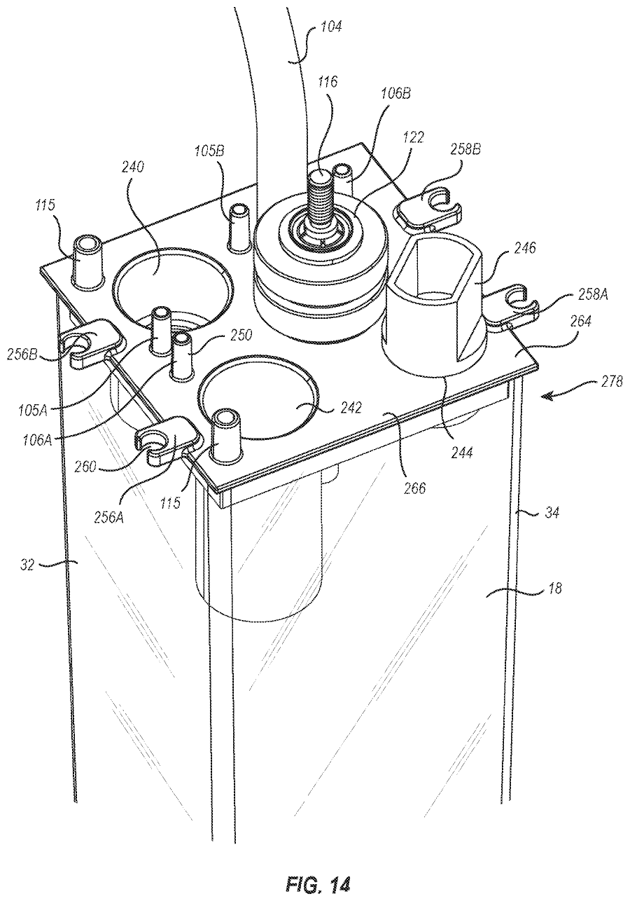

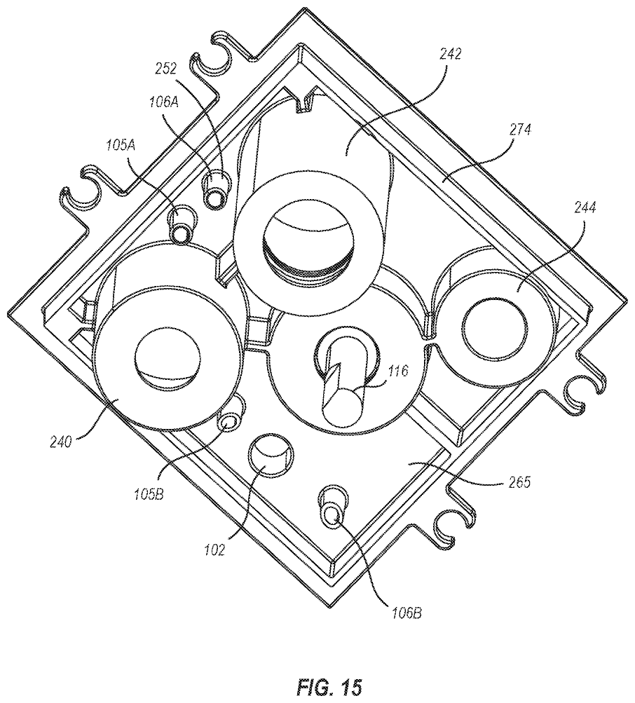

Turning to FIGS. 14 and 15, top end wall 14 is again configured for mounting to upper end 278 of encircling sidewall 18, as previously discussed. Top end wall 14 includes exterior surface 264 and opposing interior surface 265 and, more specifically, includes top panel 266 and mounting lip 274, projecting therefrom, as previously discussed. Ports 240, 242, and 244 pass through top end wall 14 and communicate with compartment 38. One or more of ports 240, 242, and 244 can optionally receive a sensor, such as previously discussed sensors 94 or 96. When not in use, ports 240, 242, and/or 244 can each be sealed by a plug 246. Upwardly projecting from exterior surface 264 of top end wall 14 are two spaced apart optional tubular stands 115 each configured to receive hanger 112 (see FIG. 11), as previously discussed. Also formed on top end wall 14 so as to pass therethrough is a first pair of ports 105A and 106A and a second pair of ports 105B and 106B. Ports 105 and 106 provide optional access to compartment 36 for delivering and/or removing the cell suspension or components thereof. Each of the ports 105 and 106 can include an upper stem 250 which projects up from exterior surface 264 of top end wall 14 and a lower stem 252 that projects down from interior surface 265 of top end wall 14. Stems 250 and 252 enable easy coupling with tubes. For example, tubes can couple with each stem 250 to extend outside of compartment 38 while separate tubes can couple with each stem 252 and extend into compartment 38. However, stems 250 and 252 are optional and other mechanisms can be used for coupling tubes to ports 105 and 106.

A port 102 extending through top end wall 14 can coupled with tube 104 and gas filter 100 (see FIG. 11), as previously discussed, to enable gas to escape from compartment 38. Centrally formed on top end wall 14 is dynamic seal 122 having at least a portion of drive shaft 116 extending therethrough. As previously discussed and depicted in FIG. 5, the upper end of drive shaft 116 couples with drive motor 126. The lower end of drive shaft 116 projects into compartment 38 and has mixing elements 158 disposed therein. During use, drive motor 126 facilitates rotation of drive shaft 116 which moves mixing elements 158 within compartment 36 for mixing the cell suspension. As previously discussed with bioreactor 10, other types of mixing systems, such as magnetic mixing systems, can be used on bioreactor 200.

Returning to FIGS. 14 and 15, a pair of spaced apart tube catches 256A and 256B are mounted on top end wall 14 so as to project past sidewall 32 while a pair of spaced apart tube catches 258A and 258B are mounted on top end wall 14 so as to project past sidewall 34. Each tube catch includes a C-shaped or J-shaped slot or hook 260 that is configured to removably receive and retain a tube, such as tubes 234 and 236 shown in FIG. 11. The tube catches can thus be used to organize, support, and/or straighten tubes used with bioreactor 200. The tube catches can also be placed at other locations and be of other shapes including open rectangles, open ovals, and similar surrounding structures.

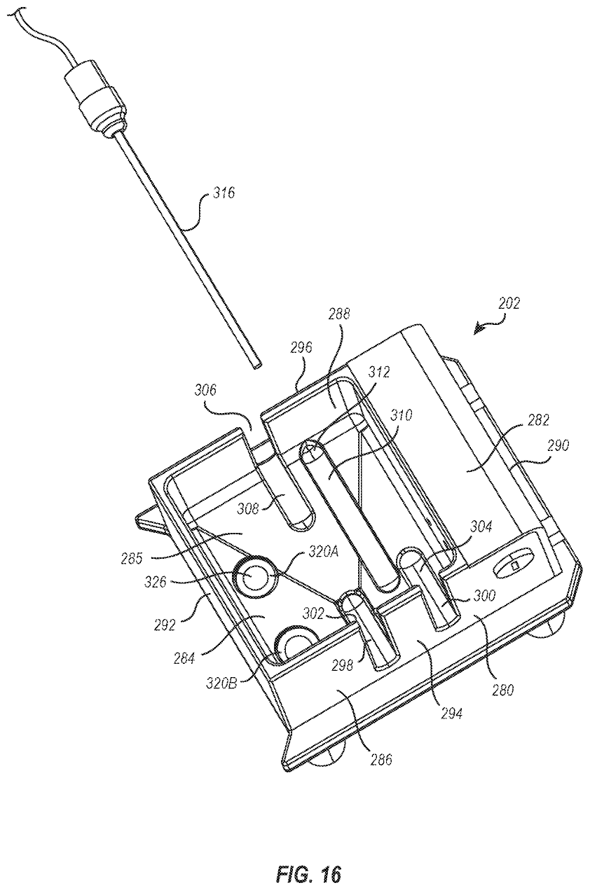

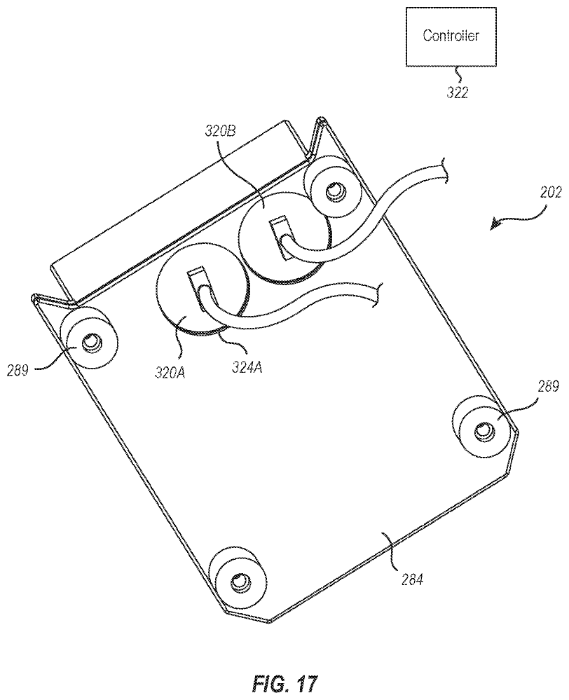

Turning to FIGS. 16 and 17, heater stand 202 comprises a body 280 having a top wall 282, an opposing bottom wall 284, and a perimeter wall 286 extending therebetween. Downwardly projecting from bottom wall 284 of body 280 are a plurality of spaced apart support legs 289 on which heater stand 202 rests. Recessed into top wall 282 so as to be at least partially encircled by perimeter wall 286 is a pocket 288. As discussed below, pocket 288 is configured to receive the lower end of housing 12A. Bottom wall 284 has an interior surface 285 that forms a floor of pocket 288. As depicted in FIG. 11, disposed within body 280 are one or more heating elements 291. More specifically, one or more heating elements 291 typically extend laterally within bottom wall 284 below pocket 288 but can also extend into perimeter wall 286. One or more heating elements 291 are typically electrical heating elements which can be powered through an electrical socket 293 (see FIG. 12) formed on body 280. A controller 295 can be used to regulate operation of one or more heating elements 291, e.g., turn on and off and control the temperature thereof. Controller 295 can be disposed spaced apart from heater stand 202 and be in electrical communication therewith by either wiring or wireless communication. Alternatively, controller 295 can be mounted directly on body 280. Body 280 or at least the portion thereof bounding pocket 288 is commonly made of metal. However, other materials that efficiently transfer heat can also be used.

Perimeter wall 286 of heater stand 202 includes a front wall 290, a back wall 292, and a pair of opposing sidewalls 294 and 296 extending therebetween that each partially bound pocket 288. As shown in FIGS. 11 and 16, a first slot 298 and a spaced apart second slot 300 pass through sidewall 294 so as to communicate with pocket 288. Slots 298 and 300 are typically linear and extend in parallel alignment through top wall 282 and project down toward bottom wall 284. A first recess 302 and a second recess 304 are recessed into interior surface 285 of bottom wall 284 and intersect with first slot 298 and second slot 300, respectively. Slots 298 and 300 can also be linear and extend in parallel alignment from sidewall 294 toward sidewall 296.

Turning to FIGS. 12 and 16, a third slot 306 passes through sidewall 296 so as to communicate with pocket 288. Third slots 306 is linear and extends through top wall 282 and projects down toward bottom wall 284. A third recess 308 is recessed into interior surface 285 of bottom wall 284 and intersect with third slot 306. Third slot 306 is linear and extends from sidewall 296 toward sidewall 294. A fourth recess 310 is also recessed into interior surface 285 of bottom wall 284. However, fourth recess 310 does no intersect with a slot. Rather, fourth recess 310 communicates with an opening 312 that passes through sidewall 296 and is completely encircled by sidewall 296. Fourth recess 310 is also linear and can extend parallel to third recess 308.

Pocket 288 is configured to receive lower end 279 of housing 12A/bioreactor 200 so that housing 12A/bioreactor 200 is securely supported in the vertical orientation, as shown in FIGS. 11 and 12. Specifically, with reference to FIGS. 11, 13, and 16, when it is desired to place housing 12A/bioreactor 200 in the vertical orientation, such as for use in the dynamic mode, lower end 279 of housing 12A/bioreactor 200 is lowered into pocket 288 so that first stem 210, second stem 216, and third stem 224 of housing 12A, each typically having a tube coupled thereto, pass down through and project out of first slot 298, second slot 300 and third slot 306 of heater stand 202, respectively. In turn, first stem 210, second stem 216, third stem 224 and fourth stem 228 of housing 12A are received within first recess 302, second recess 304, third recess 308 and fourth recess 310 of heater stand 202, respectively, as lower end 279 of housing 12A comes to rest on interior surface 285. That is, in one exemplary embodiment, interior surface 285 of heater stand 202 has a contour complementary to exterior surface 206 of bottom end wall 16 so that a relatively close tolerance fit is formed therebetween. This complementary fit aids in the stability of housing 12A/bioreactor 200 on heater stand 202 and assists in the heat transfer between heater stand 202 and housing 12A/bioreactor 200, as discussed below. Furthermore, the alignment between the various stems, slots, and recesses helps to facilitate easy and proper insertion of housing 12A/bioreactor 200 into pocket 288. It is also appreciated that bioreactor 200 can operate the static mode while mounted on heater stand 202.

With fourth stem 228 received within fourth recess 310, channel 230 within fourth stem 228 is aligned with opening 312 that passes through sidewall 296 an into fourth recess 310. With this alignment, a temperature sensor 316 can be passed through opening 312 and into channel 230 of fourth stem 228. Temperature sensor 316 detects the temperature of heater stand 202 and/or cell suspension within compartment 38. The temperature detected by temperature sensor 316 can be used to control the operation of heater stand 202, i.e., raise or lower the temperature thereof, so that the cell suspension is maintained at a desired temperature. Temperature sensor 316 can be electrically coupled with controller 295 to facilitate automatic adjustments in temperature. Alternatively, data received from temperature sensor 316 can be used to facilitate manual adjustment of controller 295. In one exemplary embodiment, temperature sensor 316 can be a resistance temperature detector (RTD). However, other types of temperature sensors can also be used.

By using temperature sensor 316 within fourth stem 228, an accurate measurement of the temperature of the cell suspension within compartment 38 can be obtained without the need for temperature sensor 316 to directly contact the cell suspension. As such, there is less risk of contamination of the cell suspension and temperature sensor 316 can be repeatedly used without any required cleaning or sterilization. Furthermore, using temperature sensor 316 within fourth stem 228 can, in some embodiments, eliminate the need to place a temperature sensor down through top end wall 14 and into compartment 38. That is, the sensors passed down through ports 240, 242, and 244 of top end wall 14, such as sensor 94 and 96, need not be a temperature sensor, thereby leaving space for other sensors or enabling the deletion of one of ports 240, 242, and 244 so as to simplify production. In still other embodiments, fourth stem 228, fourth recess 310, and temperature sensor 316 can be eliminated where a temperature sensor is used that passes down through top end wall 14 and into compartment 38.

In alternative embodiments, the placement of the various slots and recesses on heated stand can be adjusted. For example, rather than having slots 298 and 300 on sidewall 294 and slot 306 and opening 312 on sidewall 296, all three slots 298, 300 and 306 and opening 312 could be formed on either one of sidewalls 294 and 296. In other embodiments, any combination of slots 298, 300 and 306 and opening 312 can be formed on sidewalls 294 and 296. It is understood that as the slots 298, 300 and 306 and opening 312 are moved to different sidewalls 294 and 296, the corresponding recesses 302, 304, 308 and 310 are also moved so as to be in corresponding alignment therewith. Likewise, the positioning of stems 210, 216, 224 and 228 will be modified to be consistent with changes in the placement of the slots and recesses on heater stand 202. In other embodiments, any one or combination of first stem 210, second stem 216, and/or third stem 224 of housing 12A and the corresponding first recess 302, second recess 304, and/or third recess 308 with corresponding slots can be eliminated by having a corresponding inlet tube, outlet tube, and/or sparging tube pass down through top end wall 14 and into compartment 38.

With reference to FIGS. 13, 14 and 17, in one embodiment of the present disclosure, an optical sensor system can be used for detecting O₂, pH and/or CO₂ within the cell culture. The Optical sensor system generally comprises an emitter/detector 320A that is electrically coupled with a controller 322 and physio-chemical reactive spot sensor 220A or other appropriate sensor. Spot sensor 220A is mounted on the interior surface of housing 12A so that it will be in contact with the cell suspension within compartment 38. In the depicted embodiment, spot sensor 220A is mounted on the interior surface of bottom end wall 16. However, in other embodiments, spot sensor 220A can also be mounted on the interior surface of sidewall 18. Emitter/detector 320A is positioned outside of housing 12A but adjacent to and in alignment with spot sensor 220A. In the depicted embodiment, a hole 324A extends through bottom wall 284 of heater stand 202 so as to communicate with pocket 288. Hole 324A and spot sensor 220A are positioned so that when emitter/detector 320A is secured within hole 324A, a front face 326 of emitter/detector 320A is disposed adjacent to and in vertical alignment with spot sensor 220A. When used in conjunction with controller 322, emitter/detector 320A is configured to emit light through front face 326 and onto spot sensor 220A. For example, emitter/detector 320A can include an LED for emitting light. In turn, spot sensor 220A fluoresces in response to light stimulation and conditions within the cell suspension which is detected by emitter/detector 320A. The detected information is then transferred to the controller 322 which, depending on the optical sensor system being used, can determine the pH, O₂, or CO₂ within the cell suspension. Such optical sensor systems are available from PreSens Precision Sensing GmbH out of Germany.

Where needed, one, two, or three separate spot sensors can be mounted on the interior surface of housing 12A and can each be used with a corresponding emitter/detector that is electrically coupled with controller 322. Each of the separate spot sensors and emitter/detectors can be used for measuring a separate one of pH, O₂, or CO₂. For example, FIGS. 16 and 17 show the use of emitter/detectors 320A and 320B that operate with separate spot sensors 220A and 220B disposed on housing 12A and each measure a different one of pH, O₂, or CO₂. By using the spot sensors 220, prior sensors that were previously described for projecting down through top end wall 14 and into compartment 38, such as sensors 94 and 96, that would be used for measuring pH, O₂, or CO₂ can be eliminated. This help to simplify the design and production of housing 12A. Furthermore, spot sensors 220, which are relatively inexpensive, can be discarded after a single use while emitter/detectors 320 and controller 322, which do not contact the cell suspension, can be reused without the need for cleaning or sterilization.

Bioreactor 200 can be used in substantially the same way as previously discussed with bioreactor 10. For example, initially bioreactor 200, separated from heater stand 202, can be positioned within incubator 160 (see FIG. 7) in the horizontal orientation for operation in the static mode. All of the prior discussion, methods, and alternatives for operating bioreactor 10 in the static mode are also applicable to the operation of bioreactor 200 in the static mode. Thus, such prior disclosure is incorporated for bioreactor 200 but not repeated. Although bioreactor 200 does not show first mounts 80 and second mounts 82 disposed on housing 12A, the same mounts 80 and 82 and the alternatives discussed therewith can also be used on housing 12A to facilitate stacking of bioreactors 200 within incubator 160 while in the horizontal orientation. However, the positioning of mounts 80 and 82 at lower end 279 of housing 12A may need to be adjusted upward toward upper end 278 so as to enable insertion of lower end 279 within pocket 288 of heater stand 202 when in the vertical orientation/dynamic mode. Alternatively, mounts 80 and 82 could be placed at the same locations but removed prior to insertion within pocket 288. Furthermore, where stacking is not being implemented, mounts 80 and 82 are not required.

Once a predetermined time period has passed or it is detected that the cells have reached a predefined state or density or have satisfied some other predefined condition, bioreactor 200 can be operated in the dynamic mode in the vertical or horizontal orientation inside or outside of the incubator 160. In an exemplary embodiment, the bioreactor 200 is operated in dynamic mode in the vertical orientation. Again, all of the prior discussion, methods, and alternatives with regard to moving and operating bioreactor 10 in the dynamic mode (vertical or horizontal orientation) are also applicable to bioreactor 200. Thus, such prior disclosure is incorporated for bioreactor 200 but not repeated. However, in contrast to using heating jacket 420 as depicted in FIG. 20, in an alternative method of use, lower end 279 of bioreactor 200 can be fit within pocket 288 of heater stand 202. Heater stand 202 can then be used to control the temperature of the cell suspension within compartment 38. Furthermore, as discussed above, temperature sensor 316 and the discussed optical sensor system using spot sensors 220 (FIG. 13) can optionally be used to monitor the temperature, pH, O₂, and/or CO₂ of the cell suspension within compartment 38.

Depicted in FIG. 18 is a housing 12B which can be used as an alternative to housing 12A in bioreactor 200. Like elements between housing 12A and 12B are identified by like reference characters. Housing 12B is the same as housing 12A except that housing 12B includes a bottom end wall 16A that is now formed separate from sidewall 18 and is designed to be subsequently attached to sidewall 18 during assembly. Specifically, housing 12B comprises sidewall 18 that extends between upper end 278 and lower end 279. Upper end 278 has an opening 330 formed thereat that is encircled by upper edge 277. Upper edge 277 engages with top end wall 14, as previously discussed.

Lower end 279 now has an opening 332 formed thereat that is encircled by a lower edge 333. Lower edge 333/lower end 279 engages with bottom end wall 16A in substantially the same way that upper edge 277/upper end 278 engages with top end wall 14. For example, lower end wall 16A comprises a bottom panel 334. Bottom panel 334 includes interior surface 204 and exterior surface 206 having the same configuration, same components, and same alternatives as previously discussed with lower end wall 16 of bioreactor 200. Interior surface 204 and exterior surface 206 each extend to a perimeter edge 336. A mounting lip 338 upwardly projects from interior surface 204 so as to be slightly inset from perimeter edge 336 and forms a continuous loop. During assembly, mounting lip 338 is slid into opening 332 formed at lower end 279 of sidewall 18 while interior surface 204 of bottom panel 334 sits against lower edge 333 of encircling sidewall 18. Mounting lip 338 is sealed to the interior surface of encircling sidewall 18 so as to close opening 332. The sealed engagement can be achieved by welding, adhesive, press fit connection, use of a seal ring or other conventional techniques. The above configuration for bottom end wall 16 simplifies production of both bottom end wall 16A and encircling sidewall 18 and provides an easy engagement therebetween. It is appreciated, however, that bottom end wall 16A can also have a variety of other configurations for mounting at lower end 279 of encircling sidewall 18.

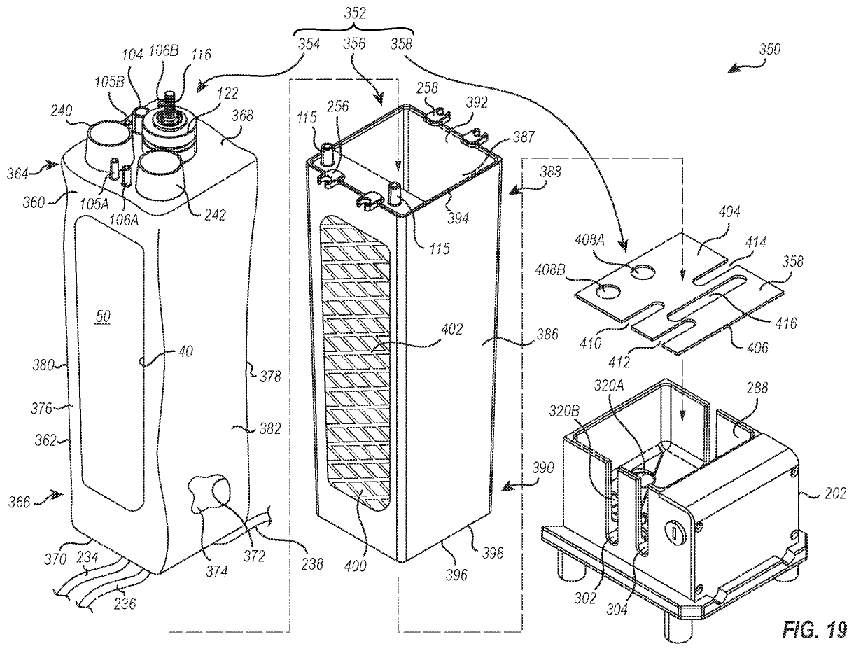

Depicted in FIG. 19 is another exemplary embodiment of a bioreactor system 350 incorporating features of the present disclosure. Like elements between bioreactor system 350 and bioreactor system 198 are identified by like reference numbers. In general bioreactor system 350 comprises a bioreactor 352 that removably couples with previously discussed heater stand 202. Bioreactor 352 comprises a bag assembly 354 which is supported within a support housing 356 and which optionally operates with a floor 358.

Bag assembly 354 comprises a flexible, collapsible bag 360 having an encircling sidewall 362 that extends from an upper end 364 to an opposing lower end 366. Upper end 364 terminates at a top end wall 368 while lower end 366 terminates at a bottom end wall 370. Bag 36 also has an interior surface 372 that bounds a compartment 374. Compartment 374 is configured to hold a fluid. Bag 360 can be formed so that compartment 374 can have any of the alternative volumes as previously discussed with regard to bioreactor 10. Likewise bag 360 can have the same alternative dimensions, e.g., height to width/diameter ratios, as previously discussed with regard to bioreactor 10. Bag 360 his comprised of one or more sheets of a flexible, water impermeable polymeric film such as a low-density polyethylene or FEP. The polymeric film can have a thickness that is at least or less than 0.02 mm, 0.05 mm, 0.1 mm, 0.2 mm, 0.5 mm, 1 mm, 2 mm, 3 mm or in a range between any two of the foregoing. Other thicknesses can also be used. The film is sufficiently flexible that it can be rolled into a tube without plastic deformation and can be folded over an angle of at least 90°, 180°, 270°, or 360° without plastic deformation.

The film can be comprised of a single ply material or can comprise two or more layers which are either sealed together or separated to form a double wall container. Where the layers are sealed together, the material can comprise a laminated or extruded material. The laminated material comprises two or more separately formed layers that are subsequently secured together by an adhesive. One example of an extruded material that can be used in the present invention is the Thermo Scientific CX3-9 film available from Thermo Fisher Scientific. The Thermo Scientific CX3-9 film is a three-layer, 9 mil cast film produced in a cGMP facility. The outer layer is a polyester elastomer coextruded with an ultra-low density polyethylene product contact layer. Another example of an extruded material that can be used in the present invention is the Thermo Scientific CX5-14 cast film also available from Thermo Fisher Scientific. The Thermo Scientific CX5-14 cast film comprises a polyester elastomer outer layer, an ultra-low density polyethylene contact layer, and an EVOH barrier layer disposed therebetween.

The material can be approved for direct contact with living cells and be capable of maintaining a solution sterile. In such an embodiment, the material can also be sterilizable such as by ionizing radiation. Examples of materials that can be used in different situations are disclosed in U.S. Pat. No. 6,083,587 which issued on Jul. 4, 2000, and United States Patent Publication No. US 2003-0077466 A1, published Apr. 24, 2003, which are hereby incorporated by specific reference.

In one embodiment, bag 360 can comprise a two-dimensional pillow style bag. In another embodiment, bag 360 can be formed from a continuous tubular extrusion of polymeric material that is cut to length. The ends can be seamed closed or panels can be sealed over the open ends to form a three-dimensional bag. Three-dimensional bags not only have an annular sidewall but also a two-dimensional top end wall and a two-dimensional bottom end wall. Three dimensional containers can comprise a plurality of discrete panels, typically three or more, and more commonly four to six. Each panel is substantially identical and comprises a portion of the sidewall, top end wall, and bottom end wall of the container. Corresponding perimeter edges of each panel are seamed together. The seams are typically formed using methods known in the art such as heat energies, RF energies, sonics, or other sealing energies.

In alternative embodiments, the panels can be formed in a variety of different patterns. Further disclosure with regard to one method of manufacturing three-dimensional bags is disclosed in United States Patent Publication No. US 2002-0131654 A1, published Sep. 19, 2002, which is incorporated herein by specific reference in its entirety.

Disposed on top end wall 368 are optionally many of the same elements as previously discussed with being disposed on top end wall 14 of bioreactor 200. Specifically, disposed on top end wall 368 are optional ports 240 and 242 that pass through top end wall 368 and communicate with compartment 374. One or both of ports 240 and 242 can optionally receive a sensor such as previously discussed sensors 94 and 96. When not in use, port 240 and/or 242 can be sealed by a plug 246 (see FIG. 14). Also formed on top end wall 368 so as to pass therethrough is first pair of ports 105A and 106A and second pair of ports 105B and 106B. Ports 105 and 106 provide optional access to compartment 374 for delivering and/or removing the cell suspension or components thereof to or from compartment 374. Ports 105 and 106 can be coupled with tubes that extend outside of compartment 374 and tubes that project into compartment 374. Port 102 extends through top end wall 368 can couple with tube 104 and gas filter 100 (see FIG. 11), as previously discussed. Centrally formed on top end wall 368 is dynamic seal 122 having at least a portion of drive shaft 116 extending therethrough. As previously discussed and depicted in FIG. 5, the upper end of drive shaft 116 couples with drive motor 126. The lower end of drive shaft 116 projects into compartment 374 and has mixing elements 158 disposed therein. During use, drive motor 126 facilitates rotation of drive shaft 116 which moves mixing elements 158 within compartment 374 for mixing the cell suspension therein. As previously discussed with bioreactor 10, other types of mixing systems, such as magnetic mixing systems, can be used on bioreactor 352.

Coupled to and extending from bottom end wall 370 are tubes 234 and 236 which can again be optionally used for delivering and/or removing the cell suspension or components thereof to or from compartment 374. Tube 238 also extends from bottom end wall 370 and can couple with sparger 222 (see FIG. 13) that is disposed within compartment 374. Tube 238 is used for delivering gas to sparger 222. Each of tubes 234, 236 and 238 can be coupled to ports mounted on bottom end wall 370 and communicating with compartment 374.

Encircling sidewall 362 can include a front wall 376, opposing back wall 378 and opposing sidewalls 380 and 382 extending therebetween. Extending through encircling sidewall 362/front wall 376 so as to communicate with compartment 374 is first transfer opening 40. In the same ways as previously discussed with bioreactor 10, gas permeable membrane 50 is secured to encircling sidewall 362/front wall 376 with or without the use of support frame 58 (see FIG. 3) so as to cover and seal first transfer opening 40. Again, all of the prior disclosure of gas permeable membrane 50 and the mounting on front wall 28 in relation to housing 12 is also applicable to bag assembly 354. In alternative embodiments, bag 360 can be formed from a gas permeable membrane, as discussed herein, so as to eliminate the need for the formation of first transfer opening 40 or the mounting of gas permeable membrane 50 thereon.

Support housing 356 comprises a support wall 386 that encircles a chamber 387 and that extends between an upper end 388 and an opposing lower end 390. Upper end 388 has an opening 392 formed thereat that communicates with chamber 387 and is encircled by a top lip 394. Lower end 390 has an opening 396 formed thereat that communicates with chamber 387 and is encircled by a bottom lip 398. Optionally secured to top lip 394 are tube catches 256 and 258. In addition, spaced apart tubular stands 115, each configured to receive a hanger 112 (see FIG. 11), as previously discussed, can optionally be secured to top lip 394.

Chamber 387 is sized and configured so as to both receive and support bag assembly 354 therein. Extending laterally through support wall 386 is an access opening 400 having a size comparable to transfer opening 40 and/or gas permeable membrane 50. Specifically, access opening 400 is sized and positioned on support wall 386 so that when bag assembly 354 is received within chamber 387, gas permeable membrane 50 is aligned with and can breathe through access opening 400. To help support bag assembly 354/gas permeable membrane 50 so that it does not excessively bulge or project out of access opening 400 while still permitting gas permeable membrane 50 to breath, support structures 402 can be formed extending across access opening 400 the directly support gas permeable membrane 50. Support structures 402 can comprise a plurality elongated rods that extend laterally, vertically, or at an angle across access opening 400. The plurality of rods can be spaced apart or partially spaced apart, e.g., they can intersect or interconnected at spaced apart locations. In one exemplary embodiment, support structures 402 can be in the form of a lattice structure. As an alternative to incorporating support structures 402, access opening 400 can be formed as a plurality of spaced apart access openings 400 that each communicate with gas permeable membrane 50. In this embodiment, the remaining portion of support wall 386 between the plurality of access openings 400 will provide support for gas permeable membrane 50. However, in any design, the access opening(s) need to be large enough to enable proper breathing of gas permeable membrane 50.

Floor 358 can be used to help support bag assembly 354 within chamber 387 of support housing 356. Floor 358 can be permanently secured to lower end 390 or removably secured to lower end 390 of support housing 356 so as to partially cover opening 396. In other embodiments, floor 358 need not be connected to lower end 390 but can simply be aligned with opening 396. In still other embodiments, floor 358 can be eliminated. Floor 358 has a top surface 404 and an opposing bottom surface. Optionally, openings 408A and 408B can extend through floor 358 so as to align with emitter/detectors 320A and 320B disposed on heater stand 202. In this embodiment, spot sensors 220A and 220B (see FIG. 13) can be mount on the interior surface 372 of bottom end wall 370 of bag 360 for use with emitter/detectors 320A and 320B, as previously discussed. Also extending through floor 358 and are slots 410 and 412 that align with recesses 302 and 304 on heater stand 202 and permit tubes 234 and 236 of bag assembly 354 to pass therethrough. Likewise, a slot 414 passes through floor 358 in alignment with recess 308 (see FIG. 16) of heater stand 202 to permit tube 238 of bag assembly 354 to pass therethrough. Finally, a slot 416 passes through floor 358 in alignment with recess 310 (see FIG. 16) of heater stand 202 to permit communication between temperature sensor 316 and bag assembly 354 when temperature sensor 316 is received within recess 310.

The bioreactor system 350 can be operated in both the static mode and the dynamic mode and in either horizontal, vertical, or angled orientation in both modes in substantially the same way as previously discussed with regard to bioreactor 200 and bioreactor 10. The only difference is that bag assembly 354 is retained within support housing 356 during both methods of operation. For example, initially bioreactor 352 with bag assembly 354 received within support housing 356 (but separated from heater stand 202) is positioned within incubator 160 (see FIG. 6) for operation in the static mode (in the horizontal orientation, vertical orientation, or angled orientation). In an exemplary embodiment, the bioreactor system 350 is oriented horizontally with membrane 50 facing down when in static mode (no mixing) and oriented vertically with sparger (not shown) operating when in dynamic mode (with mixing). All of the prior discussion, methods, and alternatives for operating bioreactor 10 in the static mode are also applicable to the operation of bioreactor 352 in the static mode. Thus, such prior disclosure is incorporated for bioreactor 352 but not repeated. Although bioreactor 352 does not show first mounts 80 and second mounts 82 disposed on support housing 356, the same mounts 80 and 82 and the alternatives discussed therewith can be used on housing 356 to facilitate stacking of bioreactors 352 within incubator 160 while in the horizontal orientation, for example. However, the positioning of mounts 80 and 82 at lower end 390 of support housing 356 may need to be adjusted upward toward upper end 388 so as to enable insertion of lower end 390 within pocket 288 of heater stand 202 when moving to the vertical orientation, such as when operating in the dynamic mode. Alternatively, mounts 80 and 82 could be placed at the same locations but removed prior to insertion within pocket 288. Furthermore, where stacking is not being implemented, mounts 80 and 82 are not required.

After an initial period of static mode cell expansion, once a predetermined time period has passed, or it is detected that the cells have reached a predefined state, density or have satisfied some other predefined condition, bioreactor 352 can be switched to and operated in the dynamic mode. Again, all of the prior discussion, methods, and alternatives with regard to moving bioreactors 10 and 200 into dynamic mode are also applicable to bioreactor 352. Thus, such prior disclosure is incorporated for bioreactor 352 but not repeated.

It is again noted that bioreactor systems 198 and 350 and the bioreactors thereof along with each of the other bioreactors disclosed herein can be operated in both the vertical orientation and the horizontal orientation within or outside of a temperature and pressure-controlled environment, such as incubator 160, can be operated in the static mode or in the dynamic mode within or outside of a temperature and pressure-controlled environment, such as incubator 160, can be operated in the static mode while in both the vertical orientation and the horizontal orientation, can be operated in the dynamic mode while in both the vertical orientation and the horizontal orientation, and can be periodically switched between the static mode and the dynamic mode while in both the vertical orientation and the horizontal orientation.

FIG. 21 is perspective view of a bioreactor 510 in accordance with example embodiments and incorporating features of the present disclosure. The bioreactor 510 depicted in FIG. 21 is similar to and contains many of the same features and components of the bioreactor 10 depicted in FIGS. 1-3. However, the bioreactor 510 of FIG. 21 does not have a gas permeable membrane 50 disposed on housing 12 or at a first transfer opening 40 (shown in FIGS. 1-3). Bioreactor 510 includes a housing 512 having a top end wall 514, an opposing bottom end wall 516, and an encircling sidewall 518 extending therebetween. The housing 512 can be made of transparent material to allow visual inspection of the contents and components within the bioreactor 510. Encircling sidewall 518 has a front wall 520 including a front face, an opposing back wall 522 including a back face, and opposing sidewalls 524 and 526 that each extend between top end wall 514 and bottom end wall 516. Sidewalls 524 and 526 also include side faces. In the depicted embodiment, encircling sidewall 518 has a rectangular or square transverse cross section. However, in other embodiments, encircling sidewall 518 can have other transverse cross section configurations such as circular, elliptical, or polygonal. In example embodiments, the housing 512 has a cuboidal shape. The curvature of each corner of the housing 512 can be made of sharp or rounded edges that have a radius of curvature or are angled in a manner that prevents biological component and cell build-up in the corners of the reactor 510. The curvature of each corner of the housing 512 can also be different to create a poka-yoke feature to assure proper orientation of the bioreactor 510.

The housing 512 has an interior surface 536 that bounds a compartment 538. Compartment 538 can have a volume of at least or less than 50 milliliters, 100 milliliters, 250 milliliters, 500 milliliters, 1 liter, 5 liters, 10 liters, 20 liters, 30 liters, 40 liters, 50 liters, or in a range between any two of the foregoing. For example, compartment 538 commonly has a volume in a range between 250 milliliters and 50 liters with between 1 liter and 20 liters or between 1 liter and 10 liters being more common. Other volumes can also be used. In one embodiment, housing 512 and the walls thereof are made from a material that is impermeable to gas and liquid, such as a polymeric material and/or biocompatible material. In other examples, the compartment can have a volume of greater than 50 liters.

Furthermore, housing 512 and the walls 514, 516 thereof are generally rigid. For example, in one exemplary embodiment, housing 512 is sufficiently rigid that it does not bow, flex and/or expand when compartment 538 is filled with liquid, such a water or cell media. The housing 512 is commonly made from a plastic such as polycarbonate, polyolefins, polyester, polystyrene, and polyacrylics and can be made of biocompatible material and produced through a molding process such as injection molding, extrusion, blow molding, 3D printing (additive manufacturing), rotational molding, or any combination thereof. Forming housing 512 from plastic also makes housing relatively inexpensive so that it can be disposed of or recycled after a single use. The rigid nature of housing 512 provides stability to bioreactor 510 and enables it to be self-supporting for proper operation during its different modes of operation, as discussed below. However, in alternative embodiments, housing 512 can be formed from a material that will have some bowing, flexing and/or expansion during use but will still be sufficiently rigid to be self-supporting. In still other alternative embodiments, as discussed further below, housing 512 can comprise a collapsible bag made of one or more sheets of polymeric film that is supported within a reusable support housing that is self-supporting, e.g., can be made of the same materials and have the same properties as housing 512, discussed above.

The housing 512 can be elongated and have a height longer than the housing width. Specifically, housing 512 has a height (e.g., H) extending between top end wall 514 and bottom end wall 516 and a width (e.g., W) or diameter. The width can extend between sidewalls 524 and 526, i.e., a width of front wall 520, or between front wall 520 and back wall 522. The height and width can vary significantly depend on the selected volume for compartment 538. In some common embodiments, height is at least or less than 0.2 meters, 0.3 meters, 0.4 meters, 0.6 meters, 0.8 meters, 1 meter or is a range between any two of the foregoing. The maximum or minimum width or diameter, in some exemplary embodiments, is commonly at least or greater than 2.5 cm, 5 cm, 7.5 cm, 10 cm, 15 cm, 20 cm, 30 cm, 40 cm, 50 cm or in a range between any two of the foregoing. In one embodiment, the maximum height is at least 1.2, 1.4, 1.6, 1.8, 2, 2.5, 3, 4, of 5 times greater than the maximum width W or diameter or is in a range between any two of the foregoing values. Again, other dimensions can also be used depending on the intended application. As discussed further below, this elongated configuration can also help optimize mixing efficiency and sparging efficiency when in the bioreactor 510 is operating in dynamic mode.

In an example embodiment, the housing 512 has approximately the following dimensions: 3.0 inches in width, 3.25 inches in depth and 13.7 inches in height, and the compartment 538 has a volume of approximately 1 liter. In another example embodiment, the housing 512 has approximately the following dimensions: 4.8 inches in width, 5 inches in depth and 21.25 inches in height, and the compartment 538 has a volume of approximately 5 liters.

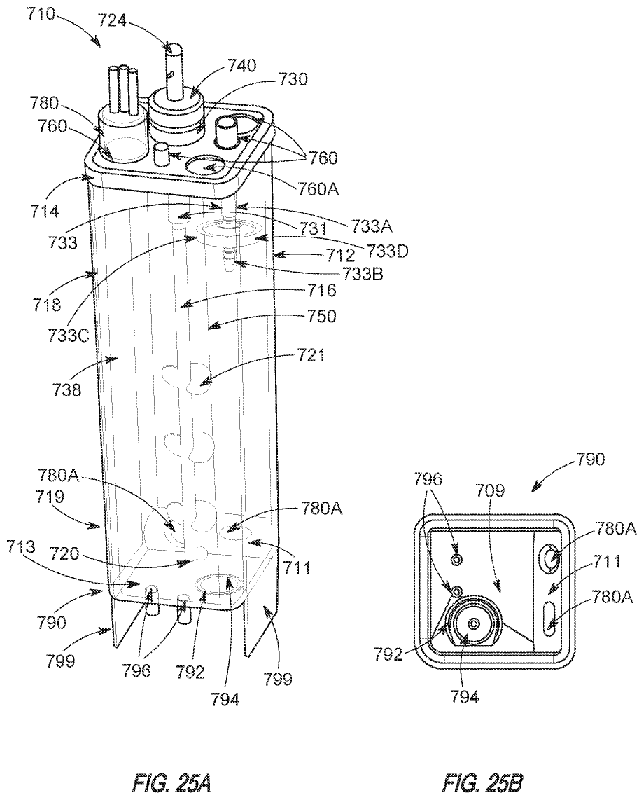

As depicted in FIG. 21, top end wall 514 is a cap that can be formed together as a unitary piece or separately from the sidewall 518. The top end wall 514 can be fixedly or removably mounted at an upper end of the bioreactor 510 housing 512. Top end wall 514 has several features, including a centered or off-set threaded bearing port 530 that can receive the top end 540 of an impeller assembly 550 and a variety of top ports 560 facilitating sterile connection with sensors 580A, dip tube and/or sensor combos 580B, tubing 580C, fluid transfer systems 580A-C, protective sensor sheaths 580A-C, top spargers or gas overlay assemblies (e.g., gas overlay assembly 733 shown in FIG. 25), or other component ports 560. The sensor sheaths can be stainless steel rods, plastic, polymer, rigid or flexible and can be used to protect or isolate a sensor from the contents of the bioreactor 510, but also allow the sensor to communicate with the contents of the bioreactor 510 through an opening. The top end wall 514 is in sealed engagement with the upper end of the bioreactor 510 housing 512. For example, the top end wall 514 can be sealed and/or connected to the top end of the bioreactor 510 housing 512 by welding, adhesive, press fit connection, use of a seal ring or other conventional techniques for sealing together and mechanically interfacing two parts. Upwardly projecting from exterior surface of top end wall 514 are spaced apart optional tubular stands 515 each configured to receive a hanger 112 (shown in FIG. 11), as previously discussed.

The bioreactor 510 depicted in FIG. 21 also includes a bioreactor base 590 formed together as a unitary piece or separately from the sidewall 518. The base 590 can be fixedly or removably mounted at a lower end of the bioreactor 510 housing 512. The base 590 contains several features, including a sparger port 592 and sparger 594 for facilitating gas transfer into the compartment 538; a variety of bottom ports 596, including tube ports 596, drain ports 596 and/or sample ports 596 for facilitating fluid transfer; sensors 580D (e.g., pressure sensors, temperature sensors, foam sensors, glucose sensors, pH sensors, DO sensors, CO₂ sensors, density sensors, cell density sensors, conductivity sensors, spot sensors or the like) for measuring fluid and process parameters; and legs 599 (e.g., cylindrical legs) that provide clearance at the bottom of the base 590. In example embodiments, sensors 580D are spot sensors positioned or fixed to the sidewalls of the bioreactor base 590. The spot sensors 580D can also be positioned or fixed to the bottom wall of the bioreactor base 590. The sparger port 592 and sparger 594 can be positioned and centered at a surface of the base 590 or off-set from center or off-set from under the impeller assembly 550 at a surface of the base 590. Examples of various types of dome spargers and film spargers that can be used in the present disclosure are disclosed in U.S. Pat. Nos. 8,603,805; 9,005,971; 9,259,692; 9,475,012; 9,682,353; 10,328,404; 9,643,133; 10,350,554; 10,843,141; and U.S. Publication No. 2021/0069654, which are incorporated herein in their entirety by specific reference herein. Other conventional spargers can also be used. The base 590 is in sealed engagement with the lower end of the bioreactor 510 housing 512. For example, the base 590 can be sealed and connected to the lower end of the bioreactor 510 housing 512 by welding, adhesive, press fit connection, use of a seal ring or other techniques for sealing together and mechanically interfacing two parts.

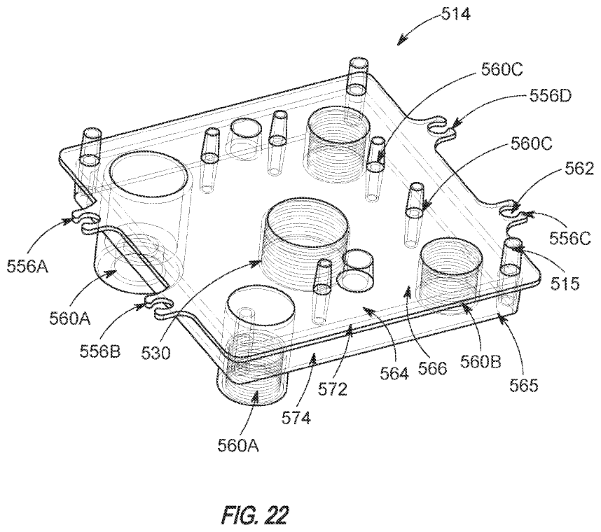

FIG. 22 depicts a top view of a top end wall or top cap 514 of the bioreactor of FIG. 21. The top end wall or top cap 514 generally comprises an exterior surface 564 and an opposing interior surface 565. Top end wall 514 is mounted at an upper end of encircling sidewall 518 so that interior surface 565 faces compartment 538. More specifically, in one exemplary embodiment, top end wall 514 comprises a top panel 566 having exterior surface 564 and opposing interior surface 565. A mounting lip 574 downwardly projects from interior surface 565 so as to be slightly inset from perimeter edge 572 and forms a continuous sub-perimeter. During assembly, mounting lip 574 is slid into an opening formed at an upper end of encircling sidewall 518 while interior surface 565 of top panel 566 sits on an upper edge of the encircling sidewall 518 of the bioreactor 510 housing 512 (shown in FIG. 21). Mounting lip 574 is sealed to the interior surface of encircling sidewall 518 (shown in FIG. 21) so as to close the opening of the bioreactor 510 housing 512. The sealed engagement can be achieved by welding, adhesive, press fit connection, use of a seal ring or other techniques. The above configuration for top end wall 514 simplifies production of both top end wall 514 and encircling sidewall 518 and provides an easy engagement therebetween. It is appreciated, however, that top end wall 514 can also have a variety of other configurations for mounting at upper end of encircling sidewall 518.

The top cap/end wall 514 of the bioreactor 510 has several features, including a central or off-set threaded bearing port 530 that can receive the top end 540 (e.g., impeller mounting hub 540) of an impeller assembly 550 and a variety of top ports 560A-C facilitating sterile connection with sensors, dip tubes, tubing, fluid transfer systems, top spargers or gas overlay assemblies, protective sensor sheaths and/or sampling or component ports. The top ports 560A-C communicate with chamber 538 (shown in FIG. 21). For example, sensors 580A and/or sensor sheaths (shown in FIG. 21) can be coupled to and/or contained within ports 560A; a dip tube and foam sensor combo 560B can be coupled to and/or contained within port 560B; and tubing 580C can be connected to ports 560C to deliver or receive fluids and/or components into and from compartment 538. Upwardly projecting from exterior surface of top end wall 514 are spaced apart optional tubular stands 515 each configured to receive a hanger 112 (see FIG. 11), as previously discussed.

In various embodiments, any combination of components 580A-C and ports 560A-C can be coupled or integrated. During operation, any one of the ports 560A-C can be left closed, opened or plugged during operation of the bioreactor 510. Any number and type of sensors 580A can used and integrated with a port 560A-C during operation, including temperature sensors, pH sensors, DO (dissolved oxygen) sensors, CO₂ sensors, glucose sensors, conductivity sensors, foam sensors, flow sensors, optical sensors, acoustic sensors, electromagnetic sensors, radar sensors or other sensors capable of detecting biological process parameters. The sensors 580A can have a variety of different configurations and can be placed in a variety of different locations on the bioreactor housing 512, making the top cap 514 customizable across a variety of components and process conditions.

One or more spaced apart tube catches 556A-D are mounted on top end wall 514 and project past top cap 514 and top panel 566. Each tube catch 556A-D includes a slot 562, C-shaped slot 562, J-shaped slot 562 or hook 562 that is configured to removably receive and retain a tube, such as tubes 234 and 236 shown in FIG. 11. The tube catches can thus be used to manage, organize, support, and/or straighten tubes used with bioreactor 510. The tube catches 556A-D can also be placed at other locations and be of other shapes including open rectangles, open ovals, and similar surrounding structures and shapes.

The bioreactor 510 of FIG. 21 and other bioreactors disclosed herein can operate in dynamic mode, with at least some level of mixing, in both the vertical (upright as shown in FIG. 21) and horizontal orientations (sideways orientation) of the bioreactor 510 and in static mode, with no mixing, in both vertical and horizontal orientations of the bioreactor 510. The bioreactors can efficiently grow cells and other biological components standing vertically or on their side horizontally with mixing, no mixing, or periods of mixing and no mixing.

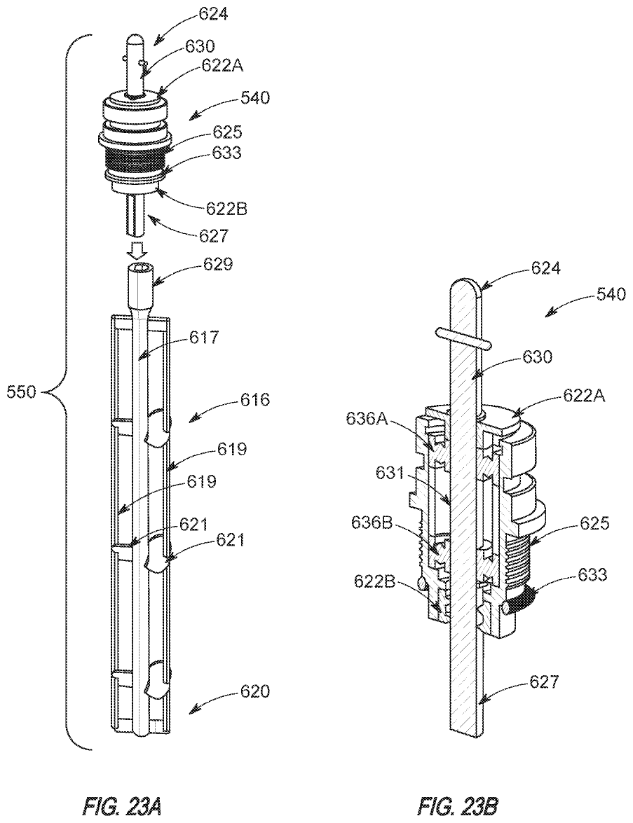

FIG. 23A depicts an example embodiment of an impeller assembly 550 that can be used to mix the contents of the bioreactors (e.g., bioreactor 10, 510, 710, 1010) disclosed herein. The impeller assembly 550 is movably and rotatably disposed within compartment 538 of the bioreactor 510 (shown in FIG. 21) for mechanically mixing biological components and other fluids within compartment 538 when the bioreactor 510 is operating in the dynamic mode. Specifically, as shown in FIG. 23A, a drive shaft 616 is provided extending between a top mount 629 and opposing second end 620. The drive shaft 616 of the impeller assembly projects from top end wall 514, into compartment 538 and towards the bottom end wall 516 (shown in FIG. 21) during operation. The drive shaft 616 can include a central shaft 617 and one or more auxiliary shafts or supports 619 in the form of rods or other structures spaced laterally apart from the central shaft 617. One or more mixing blades 621 (e.g., three sets of dual blades) can be coupled to the central shaft 617 and at least one support structure 619 of the drive shaft 616. In the depicted embodiment, three sets of dual-blades 621 are connected and coupled to the central shaft 617 and the two support rods 619 to form a multi-blade and multi-shaft mixing structure for improved mixing, uniform mixing and impeller stability.

In example embodiments, the impeller assembly 550 also includes an impeller mounting hub 540 that can be used to dynamically couple, mount and seal impeller assemblies to any of the bioreactors disclosed herein. For example, the impeller mounting hub 540 can be mechanically coupled, or screwed into the threaded bearing port 530 (or other port disclosed herein) at the top end wall 514 of the bioreactor 510 (shown in FIGS. 21, 22) to partially pass through the threaded bearing port 530 at the top end wall 514 of the bioreactor 510. A mounting shaft 630 with a terminal end 624 and a bottom mount 627 end runs and projects through the impeller mounting hub 540. The bottom mount 627 end of the impeller mounting hub 540 couples to the drive shaft 616 of the impeller assembly 550 via press fit, hex lock, or other mechanical connection. In example embodiments, the bottom mount 627 of the impeller mounting hub 540 can include a rod or other component that can couple and attach to a top mount 629 of the drive shaft 616.

A dust cover 622A is located near or proximate to the terminal end 624 of the impeller mounting hub 540 to contain bearings 636A-B (shown in FIG. 23B) and prevent dust or particles from entering the hub cavity 631 (Shown in FIG. 23B). One or more dynamic seals 622B (e.g., rotatable seal) are located near or proximate to the bottom mount 627 of the impeller mounting hub 540. In an example embodiment, the dynamic seal 622B includes two rotatable lip seals. The dynamic seal 622B seal creates fluid-tight seal (e.g., hermetic seal) at the bottom of the impeller mounting hub 540 and fluid-tight seal around a hub cavity 631 (shown in FIG. 23B). The mounting hub 540 can include a threaded portion 625 to engage and couple to threaded bearing port 530. Dynamic seal 622B can rotate and enable drive shaft 616 to rotate within the compartment 538 of the bioreactor 510 (shown in FIG. 21) while sealing the impeller mounting hub 540 and associated ports and preventing outside contaminates from passing into the mounting hub 540, port 530 or compartment 538. A terminal end 624 of the impeller mounting hub 540 projects outside of dust cover 622A and the top end wall 514 (shown in FIG. 21). The impeller mounting hub 540 of the drive shaft 116 and, more particularly, terminal end 624, couples with a drive motor (e.g., drive motor 126 shown in FIG. 5) that is typically an electric motor. The drive motor 126 of FIG. 5 or a similar drive motor can be removably mounted on the housing 512 of the bioreactor 510 (shown in FIG. 21) and, more particularly, to top end wall 514, such as through an optional tubular guard sleeve 128 (shown in FIG. 5) that encircles the impeller mounting hub 540 (also depicted as reference numeral 118 in FIG. 5). During use, a drive shaft 127 of drive motor 126 (shown in FIG. 5) can removably couple with terminal end 624 of the impeller mounting hub 540 such that activation of drive motor 126 facilitates rotation of the mounting shaft 630 and in-turn, facilitates rotation of drive shaft 616. When the drive shaft 616 is not in use, drive motor 126 and/or guard sleeve 128 (shown in FIG. 5) can be removed from housing 512 so as to minimize the size of bioreactor 510. An O-ring 633 or other seal can also be bound to or positioned on a static portion of the impeller mounting hub 540 (e.g., stator), for example near the bottom of the impeller mounting hub 540, to prevent rotation of the stator and further seal the compartment 538 and port 530 to which the impeller mounting hub 540 is coupled.

FIG. 23B depicts a cross sectional view of an impeller mounting hub 540 in accordance with example embodiments. The impeller mounting hub 540 includes a mounting shaft 630 that runs and projects through the impeller mounting hub 540. The mounting shaft 630 has a terminal end 624 and a bottom mount 627 that can mount to a drive shaft of an impeller (as described herein) in the compartment of the bioreactors disclosed herein (e.g., 10, 510 710, 1010). The bottom mount 627 of the impeller mounting hub 540 couples to a top mount 629 of the drive shaft 616 or other shafts (e.g., drive shaft 616, 716, 1018) of the impeller assemblies disclosed herein (e.g., impeller assembly 550, 750, 1016) via press fit or other mechanical connection. In example embodiments, the bottom mount 627 of the impeller mounting hub 540 can include a rod or other component with a specific shape that can couple to, be press-fit in and attach to the top mount 629 of the drive shaft 616 or any of the drive shafts disclosed herein (e.g., drive shaft/impeller shaft 616, 716, 1018) in similar fashion. A portion of the mounting shaft 630 engages with one or more bearing assemblies 636A, 636B to facilitate rotation of the mounting shaft 630. A portion of the mounting shaft 630 and one or more bearing assemblies 636A, 636B are contained and sealed within a hub cavity 631 of the mounting hub 540.

Dynamic seal 622B (e.g., rotatable seal) is located near or proximate to the bottom mount 627 of the impeller mounting hub 540. The dynamic seal 622B create a fluid-tight seal (e.g., hermetic seal) at bottom of the impeller mounting hub 540 and at least part of a fluid-tight seal around the hub cavity 631. The mounting hub 540 can include a threaded portion 625 to engage and couple to threaded bearing port 530 (or threaded bearing port 730 shown in FIG. 25). Dynamic seal 622B can rotate and enable mounting shaft 630 and one or more bearing assemblies 636A, 636B to rotate within the hub cavity 631 while sealing the impeller mounting hub 540 and associated ports and preventing outside contaminates from passing into the mounting hub 540, port 530 or compartment 538 (shown in FIG. 21). A terminal end 624 of the impeller mounting hub 540 projects outside of dynamic seal 622B. The impeller mounting hub 540, and more particularly, terminal end 624 can couples with a drive motor that is typically an electric motor as described with respect to FIGS. 5 and 23A. During use, the impeller mounting hub 540 and mounting shaft 630 can be coupled to a drive shaft 127 of a drive motor 126 (shown in FIG. 5) to rotate the mounting shaft 630, and in-turn, rotate the drive shaft 616 of an impeller assembly 550. An O-ring 633 or other seal can also be bound to or positioned on a static portion of the impeller mounting hub 540 (e.g., stator), for example near the bottom of the mounting hub 540, to prevent the start from moving/rotating and to further seal the compartment 538 and port through which the mounting hub 540 is coupled. The impeller mounting hub 540 can be used interchangeably and in conjunction with any of the mixing elements, mixers, impeller assemblies, drive shafts, and bioreactors disclosed in any of the embodiments depicted in the Figures.

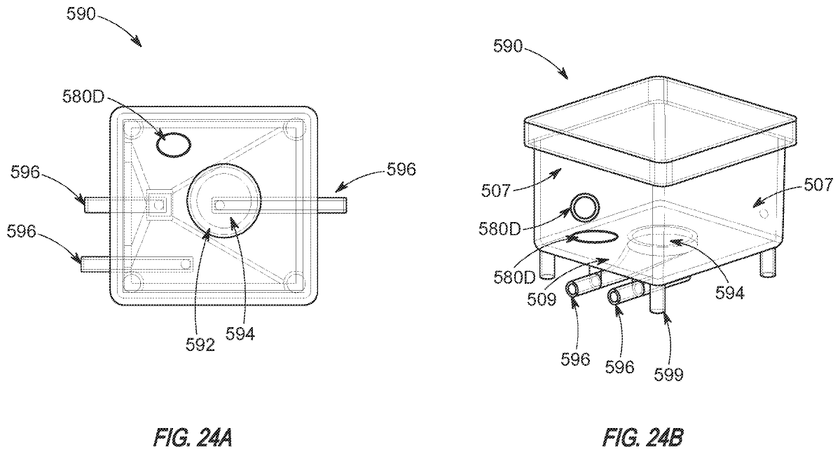

FIGS. 24A-B depicts top and perspective views of a bioreactor base 590, respectively, in accordance with example embodiments. The bioreactors disclosed herein and depicted in the Figures can include a variety of bioreactor bases, like the bioreactor base 590 depicted in FIGS. 24A-B. The bioreactor base 590 can be formed together as a unitary piece or separately from the sidewall of the bioreactor (e.g., sidewall 518 in FIG. 21). The base 590 can be fixedly or removably mounted at a lower end of the bioreactor housing (e.g., housing 512 in FIG. 21). The base 590 has sidewalls 507 and a bottom wall 509. The base 590 includes several features, including a sparger port 592 and sparger 594 for facilitating gas transfer into the compartment of the bioreactor (e.g., compartment 538 in FIG. 21); a variety of bottom ports 596, including tube ports 596, drain ports 596, sparger gas ports 596 and/or sample ports 596 for facilitating fluid transfer; sensors 580D (e.g., pH sensors, DO sensors, conductivity sensors, pressure sensors, temperature sensors, foam sensors, spot sensors or the like) for measuring fluid and process parameters; and legs 599 (e.g., cylindrical legs) that provide clearance at the bottom of the base 590. In example embodiments, the sensors 580D can be spot sensors coupled to the sidewalls 507 or bottom wall 509 of the bioreactor base 590. The sensors 580D can also be other sensors or probes coupled to ports in the base 590. The sparger port 592 and sparger 594 can be positioned and centered at a surface of the base 590 or off-set at a surface of the base 590. In example embodiments, the sparger port 592 and sparger 594 are centered under the impeller assembly (e.g., impeller assembly 550 in FIG. 21). Any of the bioreactors herein disclosed can interface with, attach to or be fit into the bioreactor base 590. For example, the base 590 can be in sealed engagement with the lower end of the bioreactor housing (e.g., housing 512 in FIG. 21). The base 590 can be sealed and connected to the lower end of the bioreactor housing (e.g., bioreactor 510 and housing 512 in FIG. 21) by welding, adhesive, press fit connection, use of a seal ring or other techniques for sealing together and mechanically interfacing two parts.

FIGS. 25A-B depict a perspective view of a bioreactor 710 and a top view of a bioreactor base 790, respectively, in accordance with example embodiments. The bioreactor 710 has many of the same features as the bioreactor 510 depicted in FIG. 21, including a housing 712 having a top end wall 714, an opposing bottom end wall 719, and an encircling sidewall 718 extending therebetween. In the depicted embodiment, encircling sidewall 718 has a rectangular or square transverse cross section. However, in other embodiments, housing 712 and encircling sidewall 718 can have other transverse cross section configurations such as circular, elliptical, polygonal. In example embodiments, the housing 712 has a cuboidal shape. In other example embodiments, the housing 712 and top end wall 714 have a poka-yoke feature with one corner having a different radius of curvature than the others as described herein.

The housing 712 has an interior surface that bounds a compartment 738. Compartment 738 can have a volume of at least or less than 50 milliliters, 100 milliliters, 250 milliliters, 500 milliliters, 1 liter, 5 liters, 10 liters, 20 liters, 30 liters, 40 liters, 50 liters, or in a range between any two of the foregoing. For example, compartment 738 commonly has a volume in a range between 250 milliliters and 50 liters with between 1 liter and 20 liters or between 1 liter and 10 liters being more common. Other volumes can also be used. In one embodiment, housing 712 and the walls thereof are made from a material that is impermeable to gas and liquid, such as media. In other examples, the compartment 738 can have a volume of greater than 50 liters.

Furthermore, housing 712 and the walls thereof are generally rigid. For example, in one exemplary embodiment, housing 712 is sufficiently rigid that it does not bow, flex and/or expand when compartment 738 is filled with liquid, such a water or media. Housing 712 is commonly made from a plastic such as polycarbonate, polyolefins, polyester, polystyrene, polyacrylics, biocompatible material and/or transparent materials and can be produced through a molding process such as injection molding, extrusion, blow molding, 3D printing (additive manufacturing), rotational molding, or any combination thereof. Forming housing 712 from plastic also makes housing relatively inexpensive so that it can be disposed of or recycled after a single use. The rigid nature of housing 712 provides stability to bioreactor 710 and enables it to be self-supporting for proper operation during its different modes of operation, as discussed below. However, in alternative embodiments, housing 712 can be formed from a material that will have some bowing, flexing and/or expansion during use but will still be sufficiently rigid to be self-supporting. In still other alternative embodiments, as discussed further below, housing 712 can comprise a collapsible bag made of one or more sheets of polymeric film that is supported within a reusable support housing that is self-supporting, e.g., can be made of the same materials and have the same properties as housing 712, discussed above. The housing 712 can be made of transparent materials to allow for visual inspection of the contents and internal components of the bioreactor 712.

In an example embodiment, the housing 712 has approximately the following dimensions: 3.0 inches in width, 3.25 inches in depth and 13.7 inches in height, and the compartment 738 has a volume of approximately 1 liter. In another example embodiment, the housing 712 has approximately the following dimensions: 4.8 inches in width, 5 inches in depth and 21.25 inches in height, and the compartment 738 has a volume of approximately 5 liters.

As depicted in FIG. 25, top end wall 714 is a lid or cap that can be formed together as a unitary piece or separately from the sidewall 718. The top end wall 714 can be fixedly or removably mounted at an upper end of the bioreactor 710 housing 712. Top end wall 714 has several features, including but not limited to, a centered or off-set threaded bearing port 730 that can receive an impeller mounting hub 740 of an impeller assembly 750 and a variety of top ports 760 facilitating sterile connection with sensors, spargers, gas injectors or overlays, dip tube and/or sensor combos, tubing, fluid transfer systems, protective sensor sheaths or other component ports 760. The sensor sheaths can be stainless steel rods, plastic, polymer, rigid or flexible and can be used to protect or isolate a sensor or sensor components from the contents of the bioreactor 710. In example embodiments a sensor assembly 780 is hermetically coupled to a top port 760 and inserted into the compartment 738 through the port 760. In example embodiments, the sensor assembly 780 couples to a PG 13.5 port 760 and includes three foam sensor rods encasing foam sensors (or level sensors) and two dip tubes for other sensors (e.g., thermocouple) inserted in the dip tubes or for directing the flow of fluids into the bioreactor 710. In other example embodiments, the sensor assembly 780 includes three foam sensor rods capable of encasing foam sensors and one dip tube with resistance temperature detector (RTD) inserted.

The top end wall 714 can additionally include a gas injector 733, such as an overhead sparger 733 or gas overlay assembly 733, fluidically coupled to a sparger or gas injector port 760A. The gas overlay assembly 733 can be connected to a gas source to facilitate flow of gas out of or into the bioreactor 710. The gas overlay assembly 733 can include a tubular conduit 733A, a gas exit nozzle 733B, a valve 733C to regulate the flow of gas through the conduit 733A and out of the nozzle 733B and a filter 733D to filter any incoming gas or particles and contaminants. For example, gas overlay assembly 733 can flow oxygen (e.g., cross flow of oxygen) to the head space of the bioreactor 710 and across the top liquid surface within the bioreactor 710 to feed and oxygenate biological components within a biological fluid (e.g., cell culture) in the bioreactor 710. The concentration of oxygen flowed to the head space of the bioreactor 710 can also control pH of the biological liquid. The gas overlay assembly 733 can also remove carbon dioxide from head space of the bioreactor 710 by connecting the gas overlay assembly 733 to a pump to pump out gas. The gas overlay assembly 733 can assure oxygen reaches the biological fluid within the bioreactor 710 when the bioreactor 710 is operating in static mode and at low turn-down ratios when the bioreactor is not full of biological liquid, such as cell culture. The gas overlay assembly 733 can feed oxygen to the bioreactor and cell culture when the cell culture is 5-90% full measured by volume of cell culture relative to the volume of the bioreactor. Overhead spargers, gas overlay assemblies, components and functionality thereof are referred to as gas delivery systems and described in detail in U.S. Pat. Nos. 9,388,375, 9,932,553, 10,519,413, 11,162,062 incorporated by reference in their entirety herein. The gas delivery systems and gas overlay assembly 733 described herein and in U.S. Pat. Nos. 9,388,375, 9,932,553, 10,519,413, 11,162,062 can be coupled to a top port 760A of any of the bioreactors described herein.

The top end wall 714 is in sealed engagement with the upper end of the bioreactor 710 housing 712. For example, the top end wall 714 can be sealed and/or connected to the top end of the bioreactor 710 housing 712 by welding, adhesive, press fit connection, use of a seal ring or other techniques for sealing together and mechanically interfacing two parts.

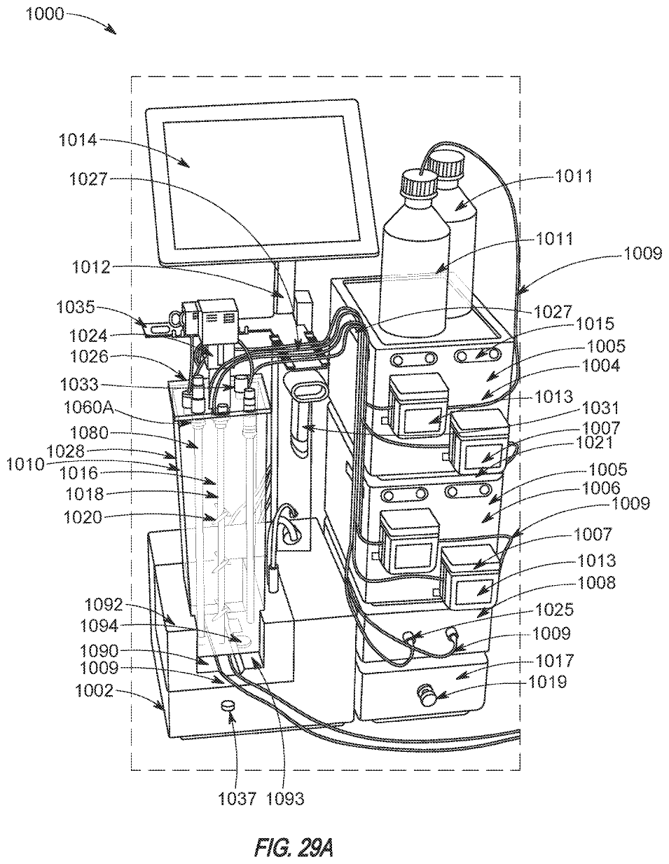

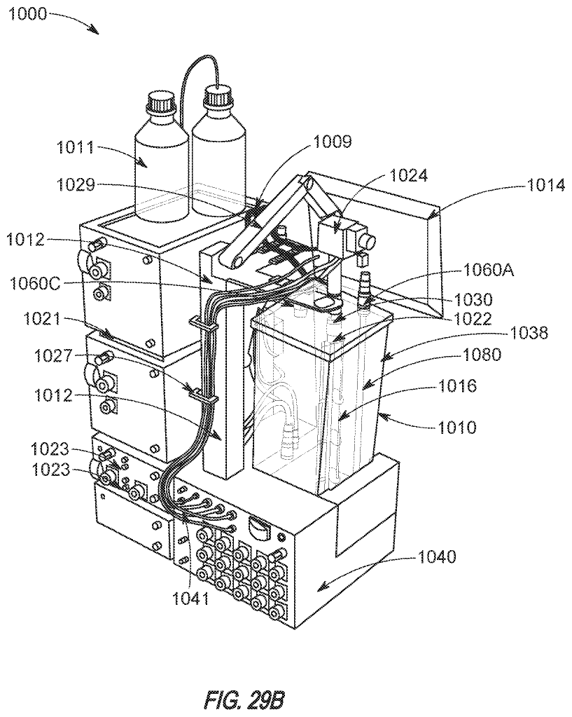

The bioreactor 710 depicted in FIGS. 25A-B also includes a bioreactor base 790 (FIG. 25B) formed together as a unitary piece or separately from the sidewall 718. The base 790 can be fixedly or removably mounted at a lower end of the bioreactor 710 housing 712. The base 790 contains several features, including a sparger port 792 and sparger 794 for facilitating gas transfer into the compartment 738; a variety of bottom ports 796, including tube ports 796, drain ports 796 and/or sample ports 796 for facilitating fluid transfer; sensors 780A (e.g., pH sensors, DO sensors, conductivity sensors, infrared temperature sensors, foam sensors, or spot sensors) for measuring fluid and process parameters; and legs 799. The legs 799 can be flat, square or rectangular legs that provide clearance at the bottom of the base 790. Each leg 799 can span the entire length of a sidewall 718 of the bioreactor 710 (e.g., shown in FIG. 25) to provide additional support and facilitate arrangement and coupling of the legs 799 into a heater stand (e.g., heater stand 202 of FIGS. 11-12) or facilitate coupling and insertion of the bioreactor 710 into a bioreactor pod base 1002 described with respect to FIGS. 29A-B. The legs 799 of the bioreactor base 790 can also sit on a surface such as a table and on top of and inside other equipment, such as an incubator.

The sparger port 792 and sparger 794 is offset from the center of a bottom wall 709 of the base 790. Offsetting the sparger 794 from the center of the base 790 and from the center of the impeller assembly 750 allows for more bubble entrainment and precludes possible impeller cavitation. The base 790 is in sealed engagement with the lower end of the bioreactor 710 housing 712. For example, the base 790 can be sealed and connected to the lower end of the bioreactor 710 housing 712 by welding, adhesive, press fit connection, use of a seal ring or other techniques for sealing together and mechanically interfacing two parts. The bioreactor base 790 includes an elevated or angled wall 711 relative to the bottom wall 709 of the base 790. The sensors 780A can be positioned or coupled to the angled wall 711 to prevent cell build-up close or proximate to the sensor 780A. Cell build-up around the sensor 780A can lead to inaccurate readings and flawed sensor measurements leading to incorrect fluid property and process parameter outputs associated with the bioreactor 710.

In example embodiments, the angled wall 711 is at an angle of between 5-70-degrees relative to a flat surface 713 of the bioreactor base 790 or relative to a horizontal plane across the bioreactor 710 when the bioreactor 710 is in the vertical orientation. In other example embodiments, the angled wall 711 is at an angle of 45-degrees relative to a flat surface 713 of the bioreactor base 790 or relative to a horizontal plane across the bioreactor 710 when the bioreactor 710 is in the vertical orientation.

In example embodiments, the drain ports 796 are vertically oriented to facilitate draining of liquids with gravity assistance. In example embodiments the drain ports 796 are 4-12 mm in length to preclude liquid hold-up or reduce liquid hold-up volume in the compartment 738 of the bioreactor 710.

The bioreactor 710 of FIG. 25A and other bioreactors disclosed herein can operate in dynamic mode, with at least some level of mixing, in both the vertical and horizontal orientations of the bioreactor 710 and in static mode, with no mixing, in both vertical and horizontal orientations of the bioreactor 710. The bioreactors can efficiently grow cells and other biological components standing vertically or on their side horizontally with mixing, no mixing, or alternating periods of mixing and no mixing.

The impeller assembly 750 of the bioreactor 710 can be used to mix the contents of the bioreactor 710 and other bioreactors disclosed herein. The impeller assembly 750 is movably disposed within compartment 738 of the bioreactor 710 for mechanically mixing biological components and other fluids within compartment 738 when bioreactor 710 is in the dynamic mode. Specifically, as shown in FIG. 25A, the impeller assembly 750 includes a drive shaft 716, mixing blade 721 and an impeller mounting hub 740 (with the same features as the mounting hub 540 described in FIGS. 23A-B). The drive shaft 716 can include three sets of tri-blades or dual-blades 721. The drive shaft 716 extends within the compartment 738 between the impeller mounting hub 740 and an opposing second end 720. Drive shaft 716 projects from top end wall 714, into compartment 738 and towards the base 790 of the bioreactor 710. The drive shaft 716 can include one or more mixing blades 721 (e.g., dual blades or tri-blades) coupled to the drive shaft 716. The blades can have the same or similar dimensions and configurations as the blades described in U.S. Pat. Nos. 9,855,537; 10,335,751; 11,654,408; 9,839,886; 10,272,400; and U.S. Patent Publication Nos. 2019/209,981 and 2021/237,009 incorporated by reference in their entirety herein.

In example embodiments, the impeller mounting hub 740 can be used to dynamically couple, mount and seal impeller assembly 750 of bioreactor 710 in the same manner described with respect to FIGS. 23A-B. For example, the impeller mounting hub 740 can be mechanically coupled, or screwed into the threaded bearing port 730 (or other port disclosed herein) at the top end wall 714 of the bioreactor 710 (shown in FIG. 25A) to partially pass through the threaded bearing port 730 at the top end wall 714 of the bioreactor 710. A mounting shaft 731 projects through the impeller mounting hub 740. The bottom end of the mounting shaft 731 couples to the drive shaft 716 of the impeller assembly 750 via press fit or other mechanical connection. The impeller mounting hub 740, more particularly, terminal end 724 can couple with a drive motor (e.g., drive motor 126 shown in FIG. 5) as described with respect to FIGS. 5 and 23A-B. In example embodiment, the mounting hub 740 of FIG. 25 has the same features and elements as mounting hub 540 of FIGS. 21, 23A-B.

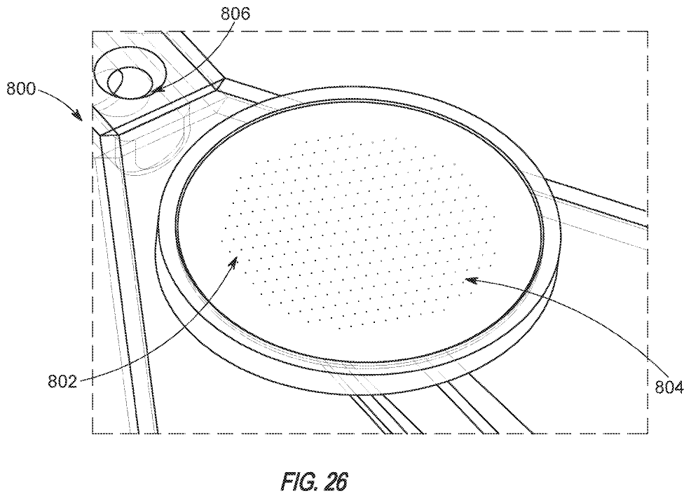

FIG. 26 is a sparger 800 in accordance with example embodiments. The sparger 800 includes sparger disc 802 with pores 804 of specific hole sizes. The holes/pores 804 in the sparger disc 802 can be laser drilled and can have the following number of holes and sizes: 360×178 μm holes; 570×178 μm holes; 760×233 μm holes; 980×368 μm holes; 1,180×445 μm holes. In examples the sparger disc 802 has a diameter from 20 mm to 60 mm, the sparger pore sizes ranging from 20 μm to 140 μm, and the sparger disc has 1-1,000 pores or holes. In other embodiments a combination of different sized holes 804 are drilled into the sparger disc 802. The sparger 800 interfaces with or includes a gas line 806 to feed gas through the sparger disc 802 and into the compartment of a bioreactor. The sparger 800 can be used with any of the bioreactors disclosed herein to transfer gases into the components of the bioreactor. Sparger can also have a non-circular geometry to better utilize and save space at the base of the bioreactors disclosed herein. The sparger 800 can also have the same features as sparger 166 or sparger 168 previously described. Examples of various types of dome spargers and film spargers that can be used in the present disclosure are disclosed in U.S. Pat. Nos. 8,603,805, 9,005,971, 9,259,692, 9,475,012, 9,682,353, 10,328,404 and 9,643,13 9,643,133, 10,350,554, 10,843,141, and U.S. Publication No. 2021/0069654, which are incorporated herein by specific reference. Other conventional spargers can also be used.

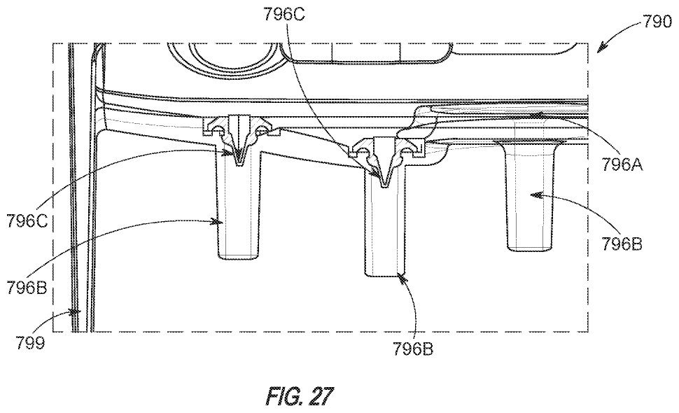

FIG. 27 depicts the bottom portion of a bioreactor base 790 in accordance with example embodiments. The bioreactor base 790 can be any of the bioreactor bases disclosed herein that have drain or sample ports, including the bioreactor base 790 depicted in FIG. 25. The bioreactor base 790 includes a drain or sample port 796A and a drain or sample tube 796B coupled to the port 796A. Biological components, and specifically, liquids from inside the bioreactor can be drained through the drain or sample port 796A and drain or sample tube 796B. A one-way valve 796C can be coupled to the port 796A or tube 796B, at the top or bottom of the port 796A or tube 796B. The one-way valve 796C allows fluid flow in only one direction through to preclude fluid from reentering the port 796A through the one-way valve 796C after fluid is discharged from port 796A and base 790 of the bioreactor 710. In example embodiments, the one-way valve 796C can be a check valve, ball valve, duckbill valve, umbrella valve or other one-way valve to prevent liquid from reentering the port 796A. The umbrella valve can be modified to allow fluid flow through in only one direction as disclosed in U.S. Pat. No. 11,598,434, issued Mar. 7, 2023, and U.S. Publication No. US2023184347A1, published on Jun. 15, 2023, which are incorporated herein by specific reference. The port, tube and valve configuration depicted in FIG. 27 reduces dead-leg space and liquid hold up volume in the base 790 of the bioreactors disclosed herein. FIG. 27 also depicts legs 799 attached to the bioreactor base 790. The legs 799 can be flat, square or rectangular legs that provide clearance at the bottom of the base 790. Each leg 799 can span the entire length of a sidewall 718 of the bioreactor 710 (e.g., shown in FIG. 25A) to provide additional support and facilitate arrangement, coupling or sliding of the legs 799 into a heater stand (e.g., heater stand 202 of FIGS. 11-12) or a bioreactor pod base 1002 (shown in FIG. 29A).

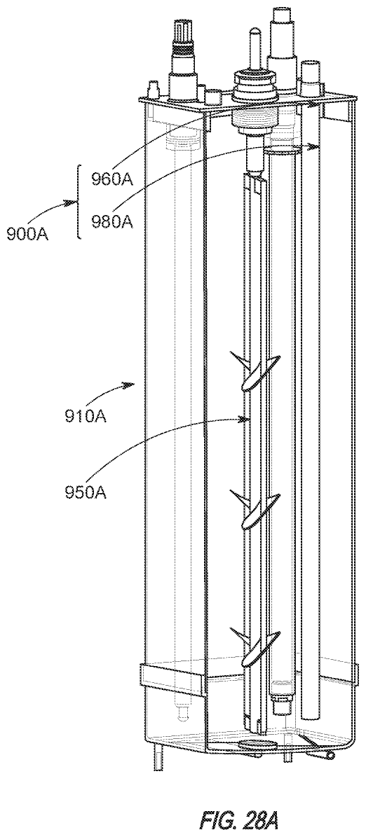

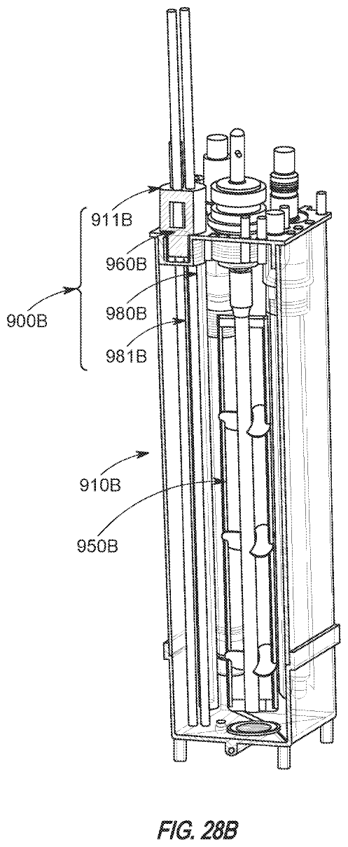

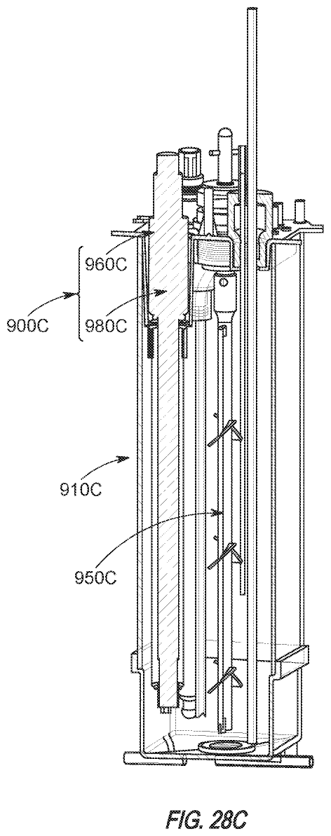

FIGS. 28A-28C depict sensor and port packs 900A-900C capable of coupling, protecting and operating a sensor at a top wall or other wall (e.g., sidewall) of the bioreactors disclosed herein. Various configurations of dual mode bioreactors 910A-C operable in static and dynamic modes are depicted in FIGS. 28A-28C. The dual mode bioreactors 910A-C have various impeller assemblies 950A-C and sensor and port packs 900A-C used during operation of the bioreactors 910A-C. Each sensor and port pack 900A-900C includes one or more ports, sensor hubs, sensor sheaths, dip tubes, sleeves and/or sensors therein. For example, sensor and port pack 900A of FIG. 28A includes a resistance temperature detector (RTD) sheath 980A coupled to a port 960A (e.g., via threaded connection) at the top wall or cap of the bioreactor 910A. A resistance temperature detector (e.g., thermocouple or temperature probe) is inserted in the sensor sheath 980A to isolate the sensor/detector from the contents of the bioreactor 910A while temperature measurements are generated by the sensor or probe thereof from an opening in the sensor sheath 980A.

Sensor and port pack 900B of FIG. 28B includes a sensor hub 911B coupled to a sensor port 960B (e.g., via threaded connection) at the top wall or cap of the bioreactor 910B. The sensor hub 900B includes at least two holes. A dip tube 980B and a foam sensor 981B within the dip tube 980B are inserted into each hole. The foam sensor 981B (e.g., level sensor) can communicate with the contents of the bioreactor 910B through an opening at the bottom of the dip tube 980B to sense the build-up, volume and/or height of foam in the bioreactor 910B. The dip tube 980B can contain a sensor or provide a fluid path to flow and discharge fluids/liquids into specific discharge locations within the bioreactor 910B.

Sensor and port pack 900C of FIG. 28C includes a port 960C and a dissolved oxygen sleeve 980C coupled to the port 960C (e.g., via threaded connection) at the top wall or cap of the bioreactor 910C. A dissolved oxygen sensor is inserted in the sleeve 980C to isolate the sensor from the contents of the bioreactor 910C while dissolved oxygen measurements are generated by the sensor near the bottom of the sleeve 980C (e.g., though an opening in the sleeve 980C). The sensor sheaths 980A, sleeves 980C and dip tubes 980B can be made from rigid or flexible material, including rigid or flexible tubing or hollow rods made of steel, stainless steel, polymeric material or other metal, plastic or medical grade/surgical/sterile materials. The sensor sheaths 980A, sleeves 980C and dip tubes 980B can also include openings in the sheaths/sleeves/tubes to facilitate communication between the sensors and the contents of the dual mode bioreactors 910A-C.

The dual mode bioreactors 910A-C of FIGS. 28A-C and other bioreactors disclosed herein can operate in dynamic mode, with at least some level of mixing, in both the vertical and horizontal orientations of the bioreactors 910A-C and in static mode, with no mixing, in both vertical and horizontal orientations of the bioreactors 910A-C. The bioreactors 910A-C can efficiently grow cells and other biological components standing vertically or on their side horizontally with mixing, no mixing, or periods of mixing and no mixing to customize mixing, gassing, sparging, and other operations for cell therapy, gene therapy, antibody production or other applications for growing and producing biological components.

FIGS. 29A-B depict front and back views of a bioreactor pod 1000 respectively, in accordance with example embodiments. The reactor pod 1000 can include a pod base 1002; a dual mode bioreactor 1010 (e.g., any of the bioreactors described herein) insertable into the pod base 1002, one or more portable and arrangeable pod modules 1004, 1006, 1008, 1017; a stand 1012 for mounting pod components; a display 1014 including a user interface for transmitting user inputs and receiving process parameter outputs from and associated with the bioreactor pod 1000, bioreactor 1010, pod modules 1004, 1006, 1008, 1017 and pod components; a motor 1024; sensors 1080; and a controller 1037 with memory and a processor located in the pod base 1002 and used for controlling the bioreactor 1010, motor 1024, pod modules 1004, 1006, 1008, 1017 and other pod 1000 components. In other embodiments, the controller 1037 can be a separate unit from the pod base 1002 and located remote from the bioreactor pod 1000.

User inputs associated with the bioreactor 1010 or bioreactor pod 1000 can include, but are not limited to set points or user inputs indicating fluid temperature, system pressure, impeller speed, motor RPM, gas flow rate into or out of the bioreactor, dissolved oxygen concentration in the liquid (e.g., cell culture media), pH of the liquid, liquid flow rate into or out of the bioreactor; gas flow rate through a bottom sparger; gas flow rate through an gas overlay assembly; pump speed or RPM, pump direction, bar codes scanned by scanner 1031, and/or operational pod/bioreactor recipes that control the duration of static and dynamic growing times/conditions in the bioreactor and that control bioreactor pod components, such as pod modules 1004, 1006, 1008, 1017. All or some of these inputs can be stored in a workflow or pod recipe that is selected through an operator input at the display 1014 and user interface and run by the controller 1037 processor as described herein.

Process parameter outputs associated with the bioreactor 1010 and bioreactor pod 1000 can include, but are not limited to, sensor measurements, data or signals; impeller speed; motor RPM; gas flow rate into or out of the bioreactor, liquid flow rate into or out of the bioreactor; gas flow rate through a bottom sparger; gas flow rate through an gas overlay assembly; pump speed or RPM; and/or scanner data (e.g., scanner 1031 described below). A variety of sensors (e.g., sensor 1080) can be coupled to the bioreactor 1010, including but not limited to, pressure sensors, temperature sensors, foam sensors, glucose sensors, pH sensors, DO sensors, CO₂ sensors, flow sensors, density sensors, cell density sensors, conductivity sensors, spot sensors or the like. Sensor measurements, data or signals transmitted from sensors (or associated transmitters) are liquid and system parameter outputs, such as pressure within the bioreactor, temperature within the bioreactor or cell culture; foam content within the bioreactor; glucose content within the bioreactor or cell culture; pH within the bioreactor or cell culture; dissolved oxygen content within the bioreactor or cell culture; CO₂ content within the bioreactor or cell culture; cell density of the cell culture; conductivity readings associated with the bioreactor or cell culture.

The bioreactor 1010 is a dual mode bioreactor 1010 that can operate in static mode (with no mixing) and dynamic mode (with at least some mixing or agitation) to support a biologically active environment and conduct biological processes, such as seed train and cell expansion applications. The time period and alternating duration of static versus dynamic mode operation of the bioreactor 101 can be stored as recipes in the memory of controller 1037 of the bioreactor pod 1000 to optimize cell growth and preservation.

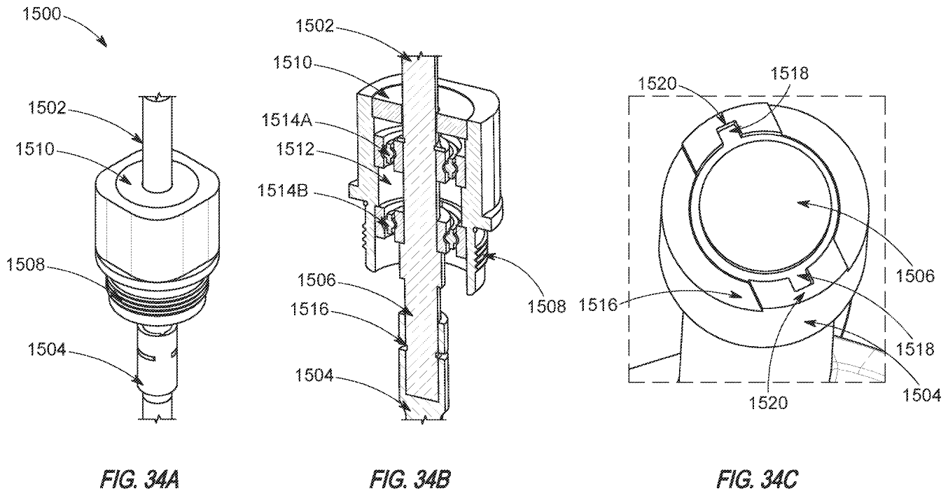

In example embodiments, the bioreactor 1010 can include the same features and components as the bioreactor 710 depicted in FIG. 25. In this example of a bioreactor pod 1000, the bioreactor 1010 includes an impeller assembly 1016 with one impeller shaft 1018 (or drive shaft) and three sets of three impeller blades 1020 (or tri-blades) spaced vertically apart and coupled to three sections of the impeller shaft 1018, resulting in nine total blades coupled to the impeller shaft 1018. In example embodiments, one to six sets of tri-blades 1020 are spaced vertically apart and coupled to one or more sections of the shaft 1018. The impeller assembly 1016 can also include an impeller mounting hub 1022 (shown in FIG. 29B) to secure and couple one end of the impeller shaft 1018 to a motor 1024. In example embodiments, the impeller shaft 1018 can be coupled to an electrical motor 1024 and/or associated motor shaft via the impeller mounting hub 1022. The motor 1024 rotates a shaft of the impeller mounting hub 1022, which in turn rotates the impeller shaft 1018 and tri-blades 1020 of the impeller assembly 1016 during operation of the bioreactor 1010 in dynamic mode. A motor mount 1035 is attached to the stand 1012. The motor mount 1035 can be used to mount the motor 1024 when it is decoupled from the impeller mounting hub 1022. The impeller mounting hub 1022 can be any of the impeller mounting hubs described herein (e.g., FIGS. 23A-B, 25). In an example embodiment, the impeller mounting hub 1022 has the same components, structure and functionality of the impeller mounting hub 1500 depicted in FIGS. 34A-C described herein.

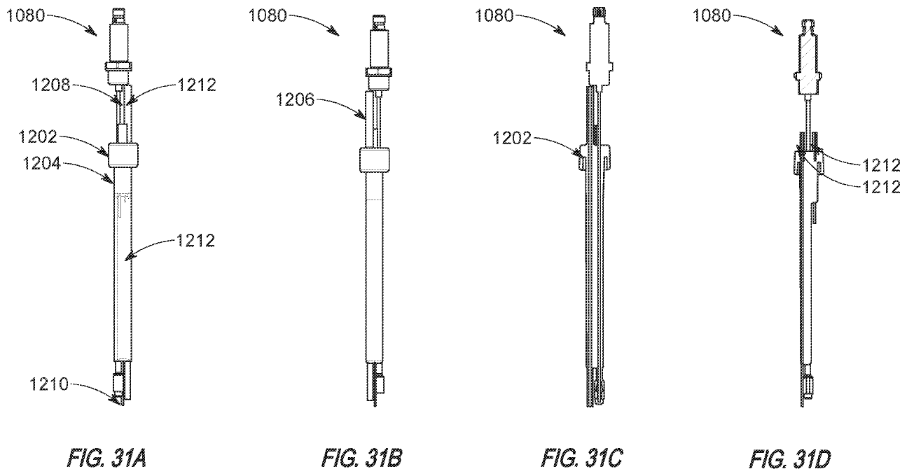

The top end wall 1026 of the bioreactor 1010 is a lid or cap that can be formed together as a unitary piece or separately from the housing 1028 of the bioreactor 1010 as described with respect to FIG. 25. The top end wall 1026 can be fixedly or removably mounted at an upper end of the bioreactor housing 1028. Top end wall 1026 has several features, including but not limited to, a centered or off-set threaded bearing port 1030 that can receive an impeller mounting hub 1022 of an impeller assembly 1016 and a variety of top ports 1060A, 1060C facilitating sterile connection with sensors, spargers, gas overlay assemblies, dip tube and/or sensor combos, tubing, fluid transfer systems, protective sensor sheaths or other component ports 1060A, 1060C. The sensor sheaths can be stainless steel rods, plastic, polymer, rigid or flexible and can be used to protect or isolate a sensor from the contents of the bioreactor 1010, but also provide an opening for sensors to communicate with the contents of the bioreactor 1010. In example embodiments a sensor assembly 1080 is hermetically coupled to a top port 1060A and inserted into the bioreactor compartment 1038 through the port 1060A. In example embodiments, the sensor assembly 1080 (or sensor pack) couples to a PG 13.5 port 1060A and includes three foam sensor rods encasing foam sensors and two dip tubes. In other example embodiments, the sensor assembly 1080 includes three foam sensor rods capable of encasing foam sensors, one dip tube, one RTD tube and a barb and cable tie connection to couple the sensor pack to the cap 1026 of the bioreactor 1010. In example embodiments, the sensor assembly (or sensor pack), is the sensor assembly 1080 described with respect to FIGS. 31A-D.

An overhead sparger or gas overlay assembly 1033 (shown in FIG. 29A) can be fluidically coupled to a gas port 1060C (Shown in FIG. 29B). The gas overlay assembly 1033 can be connected to a gas source through gas flow module 1008 to facilitate flow of gas out of or into the bioreactor 1010. The gas overlay assembly 1033 can include a tubular conduit, a gas exit nozzle, a valve and a filter (as shown in FIG. 25) to regulate the flow of gas through the conduit and out of the nozzle. For example, gas overlay assembly 1033 can flow oxygen (e.g., cross flow of oxygen) to the head space of the bioreactor 1010 and across the top liquid surface within the bioreactor 1010 to feed and oxygenate biological components within a biological fluid (e.g., cell culture) in the bioreactor 1010. The concentration of oxygen flowed to the head space of the bioreactor 1010 can also control pH of the biological liquid. The gas overlay assembly 1033 can also remove carbon dioxide from head space of the bioreactor 1010 with and through pod module 1008 or by connecting the gas overlay assembly 1033 to a pump to pump out gas. The gas overlay assembly 1033 can assure oxygen reaches the biological fluid within the bioreactor 1010 when the bioreactor 1010 is operating in static mode or dynamic mode and at low turn-down ratios when the bioreactor is not full of biological liquid, such as cell culture media. The gas overlay assembly 1033 can feed oxygen to the bioreactor 1010 and cell culture when the bioreactor 1010 is 5-90% full of cell culture media as measured by volume of cell culture relative to the volume of the bioreactor 1010. Gas overlay assemblies 1033, overhead spargers, components and functionality thereof are referred to as gas delivery systems and described in detail in U.S. Pat. Nos. 9,388,375, 9,932,553, 10,519,413, 11,162,062. The gas delivery systems, overhead spargers and gas overlay assemblies 1033 described herein and in U.S. Pat. Nos. 9,388,375, 9,932,553, 10,519,413, 11,162,062 can be coupled to a top port 1060C of any of the bioreactors described herein.

The bioreactor 1010 also includes a bioreactor base 1090 that has the same or similar components, features and functionality of the bioreactor base 790 described with respect to FIGS. 25A-B and 27. As shown more clearly in FIGS. 25A-B and 27, the bioreactor base 1090 has legs 799 (shown in FIGS. 25, 27) that can slide in and couple to a bioreactor receiver 1092 of a bioreactor pod base 1022 to secure the bioreactor 1010 to the bioreactor pod 1000. The bioreactor base 1090 also has a bottom sparger 1094 to flow gas (e.g., oxygen) to the contents of the bioreactor 1010 when the bioreactor is operating in dynamic or static mode. The bottom sparger 1094 can be any of the spargers described herein, including but not limited to, spargers 166 and/or 168 described with respect to FIG. 8; sparger 222 described with respect to FIG. 13; sparger 594 described with respect to FIGS. 21, 24; sparger 794 described with respect to FIG. 25; or sparger 800 described with respect to FIG. 26. Examples of various other spargers that can be used in the present disclosure and as sparger 1094 are disclosed in U.S. Pat. Nos. 9,005,971 and 9,643,133, which are incorporated herein by specific reference. Other conventional spargers can also be used. The bottom sparger 1094 can be off set from the center of the bioreactor base 1090 as described with respect to FIG. 21 and sparger 594 or can be centered under the impeller assembly 1016. Offsetting the sparger 1094 from directly beneath the impeller assembly 1016 can prevent bursting and/or coalescing of sparge bubbles which can damage or kill cells and inhibit cell growth.

The pod base 1002 can include many of the same features as the heater stand 202 described with respect to FIGS. 11, 12, 16 and 17. The pod base 1002 includes a controller 1037 with memory and a processor located in the pod base 1002 and used for controlling the bioreactor 1010, motor 1024, pod modules 1004, 1006, 1008, 1017 and other pod 1000 components. In other embodiments, the controller 1037 can be a separate unit from the pod base 1002 and located remote from the bioreactor pod 1000. The pod base 1002 also includes heating elements (not shown) identical or similar to the heating elements 291 of heater stand 202 described with respect to FIG. 11. The heating elements 291 along with the bioreactor receiver 1092 form a heater that can heat the contents of the bioreactor 1010. The heating elements can be resistive heating elements, conductive heating elements or other heating elements and the bioreactor receiver 1092 can be made of conductive material (e.g., aluminum, titanium or other conductive material) that conducts heat from the heating elements to the bioreactor base 1090 and contents of the bioreactor 1010. In example embodiments, the heating elements are incorporated in the bioreactor receiver 1092. The bioreactor receiver 1092 can be shaped as a mold of part of the bioreactor 1010 housing 1028 to optimally interface with and hold the bioreactor 1010. The bioreactor receiver 1092 can also have an opening 1093 that the bioreactor 1010 can slide in to mount the bioreactor 1010 to the pod base 1002. The opening 1093 also provides visibility to the bioreactor 1010 and contents of the bioreactor 1010 through the transparent housing 1028 of the bioreactor 1010.

The pod base 1002 can also include a power supply unit 1040 that can supply power through cables 1041 to components of the bioreactor pod 1000, including but not limited to, pod modules 1004, 1006, 1008, 1017, motor 1024, and sensors 1080; and a controller 1037. The cables 1041 can transmit power and/or data to and from components of the bioreactor pod 1000, including but not limited to, pod modules 1004, 1006, 1008, 1017, motor 1024, and sensors 1080, and a controller 1037. Control, sensor and component signals and data can also be transmitted wirelessly between components of the bioreactor pod 1000 and the controller 1037 to control the operation of the bioreactor pod 100 and components thereof. The power supply unit 1040 can include one or more AC power supplies, DC power supplies, power distribution boxes, programmable power supplies, uninterruptible power supplies, switched mode power supplies and/or other power supplies.

The pod modules 1004, 1006, 1008, 1017 are modular components, each a separate unit or one or more combined units, that are portable, stackable, and arrangeable in a variety of configurations. The pod modules 1004, 1006, 1008, 1017 can be stacked on top of each other in any order. The pod modules 1004, 1006, 1008, 1017 can also be arranged individually on a surface without stacking. The pod modules 1004, 1006, 1008, 1017 can be positioned to the right or the left of the bioreactor 1010 and pod base 1002 in stacked fashion or individually in unstacked fashion. One or more of the pod modules 1004, 1006, 1008, 1017 can be positioned to the left of the bioreactor 1010 and pod base 1002 at the same time as one or more of the pod modules 1004, 1006, 1008, 1017 is positioned to the right of bioreactor 1010 and pod base 1002. The pod modules 1004, 1006, 1008, 1017 include indents 1021, a recessed portion 1021 or a ledge 1021 to allow a user to easily handle, lift, move and arrange the pod modules 1004, 1006, 1008, 1017 in any stacked or unstacked configuration or position. Each of the pod modules 1004, 1006, 1008, 1017 and/or associated pod transmitters can communicate with, transmit data to and receive data and control signals from the controller 1036 to control operation of the of the pod modules 1004, 1006, 1008, 1017. Data and signals can be transmitted between the pod modules 1004, 1006, 1008, 1017 and controller 1037 wirelessly via wireless transmitters or with wired transmission via cables 1041.

The bioreactor pod 1000 can include any number of pod modules, including for example, 1-10 pump modules to pump fluids to/from pod equipment including bioreactors, 1-10 mass flow controller modules to control the flow of fluids to/from pod equipment; 1-10 electrical modules to power pod equipment; 1-10 equipment control modules to control pod equipment; 1-10 anti-foam modules to deploy foam control measures; 1-10 sensor transmitter modules that receive, process and transmit sensor data, signals and measurements; 1-10 emergency stop modules that can govern and cut power to pod equipment; 1-10 heater modules for heating pod equipment including bioreactors; and/or other modules stacked, arranged and customized for the specific expansion process, and particularly cell expansion for cell and gene therapy applications. Each pod module can have a separate housing with indents to ease handling and porting of pod modules in stacked and unstacked configurations proximate to or remote from the bioreactor pod base. One or more pod modules and associated functionality can also be combined under a unified housing. Pod modules combined under a unified housing can be stacked, arranged and/or customized for ease of use and for specific biological expansion processes, such as cell and gene therapy processes described herein.

In the example embodiment depicted in FIGS. 29A-B, pod module 1004 and pod module 1006 are first and second pump modules 1004, 1006. The first pump module 1004 and the second pump module 1006 each have a housing 1005 that house two pump heads 1013 and two pumps 1007 each. The four total pumps 1007 can be any type of pumps, including peristaltic pumps. One or more of the pump modules 1004 and associated pumps 1007 are fluidically connected to the bioreactor 1010 and one of the liquid containers 1011 via tubing 1009 to pump fluid between the container 1011 and the bioreactor 1010 via tubing 1009. The pump modules 1004 can also be connected to other containers or equipment to pump liquids to and from the equipment or containers. In other embodiments, the pump modules 1004, 1006 can include less than two pumps and pump heads or more than two pumps and pump heads each. The bioreactor pod 1000 can also include more or less than two pump modules. Each pump 1007 can also be electrically connected to a pump actuation button 1015 that can actuate the pump to flow liquid towards the container 1011 or towards the bioreactor 1010, or alternatively, the pump actuation button (e.g., with two buttons) can cause the pumps 1007 to flow fluid left or right of the pumps 1007 and to and from other containers or equipment depending on which of the two inputs are actuated on the pump actuation buttons 1015. The pump modules 1004, 1006 and flow rate and direction of fluid flow pumped from the pumps 1007 can be controlled by the controller 1037 based on user inputs and at the display 1014 and user interface and recipes stored in memory of the controller 1037. Any type of fluid can be pumped and flowed to and from bioreactor 1010 and containers 1011 via pump modules 1004 and 1006 and pumps 1007, including biological fluids, cells, cell media water, and/or one or more biocomponents, fluids, solids, mixtures, solutions, and suspensions including, but not limited to, bacteria, fungi, algae, plant cells, animal cells, white blood cells, T-cells, cell media, protozoans, nematodes, plasmids, viral vectors, blood, plasma, organelles, proteins, nucleic acids, lipids, plasmids, carbohydrates, and/or other biological components, and the like. Examples of some common biological components include E. coli, yeast, bacillus, and CHO cells. Fluids can also include cell-therapy cultures and cells and microorganisms that are aerobic or anaerobic and adherent or non-adherent.

In the embodiment depicted in FIGS. 29A-B, pod module 1008 is a mass flow controller module 1008 (MFC module 1008) that controls the flow of fluid through the MFC module 1008, the tubing 1009 and to the bioreactor 1010. In example embodiments, the MFC module 1008 controls the flow of gas to the gas overlay assembly 1033 and the bottom sparger 1094. Gases, such as oxygen, can be routed and flowed into the bioreactor 1010 through the gas overlay assembly 1033 and the bottom sparger 1094 to support a biologically active environment and expand cell growth and proliferation in the bioreactor 1010 when the bioreactor is operating in static or dynamic mode. The MFC module 1008 and flow rate and direction of fluid flow (e.g., oxygen gas flow) through the MFC module 1008 and to the bioreactor 1010 can be controlled by the controller 1037 based on user inputs and at the display 1014 and user interface and recipes stored at memory of the controller 1037. In example embodiments, the MFC module 1008 includes four gas inlets 1023 and two gas outlets 1025 that are fluidly connected to tubing 1009. Two of the inlets 1023 feed gas through one outlet 1025 and tubing 1009 to the gas overlay assembly 1033, and the other two inlets 1023 feed gas through one outlet 1025 and tubing 1009 to the bottom sparger 1094. Other gas outlet and inlet configurations are also possible.

In the embodiment depicted in FIGS. 29A-B, pod module 1017 can be an optional module that is integrated into the pod base 1002 or a separate electrical module 1017 that supplies power through cables 1041 to components of the bioreactor pod 1000, including but not limited to, pod modules 1004, 1006, 1008, motor 1024, sensors 1080, and the controller 1037. The cables 1041 can transmit power and/or data to and from components of the bioreactor pod 1000, including but not limited to, pod modules 1004, 1006, 1008, motor 1024, and sensors 1080, and a controller 1037. The electrical module 1017 can include one or more AC power supplies, DC power supplies, power distribution boxes, programmable power supplies, uninterruptible power supplies, switched mode power supplies and/or other power supplies. The electrical module 1017 can also include an emergency stop 1019 that cuts power and/or stops operation of one or more components of the bioreactor pod 1000, including pod modules 1004, 1006, 1008, motor 1024, and sensors 1080, and a controller 1037. In example embodiments, the electrical module 1017 is part of the reactor pod base 1002 as described previously.

The stand 1012 and pod modules 1004, 1006, 1008, 1017 can include a variety of tubing 1009 and cable 1041 management clips 1027 or slots 1027 to manage, organize and arrange the tubing 1009 and cables 1041 in a manner that does not interfere with access to, operation of, movement of, or actuation of any of the components of the bioreactor pod 1000. Tubing 1009 and cable 1041 management clips 1027 can be positioned on one or more sidewalls of the pod modules 1004, 1006, 1008, 1017 and on one or more surfaces or platforms of the stand 1012. The stand 1012 can include a moveable arm 1029 attached to the display 1014 to facilitate upward, downward, and sideways movement of the display 1014 and user interface to convenient locations for operator input and output display.

The stand 1012 can also include a scanner 1031, such as a bar code scanner 1031, that can scan serial numbers or bar codes associated with contents or components of the pod 1000, including serial numbers or bar codes associated with the bioreactor 1010, sensors 1080, pod modules 1004, 1006, 1008, 1017, pod base 1002, bioreactor receiver 1092, gas overlay assembly 1033, sparger 1094 or other components. Upon scanning with the scanner 1031, the bar code or serial number provides data about the component, including but not limited to, data identifying the bioreactor 1010 type, size, impeller type, manufacturer, and manufacturing date; sensor 1080 type, calibration data, manufacturer, and manufacturing date; pod module 1004, 1006, 1008, 1017 type, capacity, calibration data, manufacturer, and manufacturing date; bioreactor receiver 1092 type, size, manufacturer, and manufacturing date; gas overlay assembly 1033/sparger 1094 size, pore size, gas flow rate capacity, material of construction, manufacturer, and manufacturing date; and other parameters and details about pod 1000 components. This scanned data can be stored in the memory of the controller 1037 and used in pod recipes that include a set of instructions to control the operation, flow rates, impeller speeds, reactor operating modes (e.g., static versus dynamic) and/or periods or length of time for each bioreactor operating mode. Pod operational recipes can be stored in the controller 1037 memory and recalled and run by the controller's processer based on the equipment type, including but not limited to bioreactor, gas overlay assembly, sparger, impeller or sensor type and/or manufacturer, and equipment parameters, including but not limited to bioreactor volume/capacity, overhead gas flow rate capacity, sparger pore size and membrane type, impeller size and blade type, or sensor calibration data.

The controller 1037 can include one or more processors and memory for running pod recipes, in the form of machine code instructions, to automate one or more components of the bioreactor 1010 and bioreactor pod 1000. One or more bioreactor or pod automation recipes can be stored in the memory of the controller 1037 and run via input by an operator via display 1014 and associated user interface. The time period, number of times and sequence that the bioreactor 1010 operates in dynamic and static mode during expansion steps and associated impeller speeds and gas flow rates to gas overlay assemblies and spargers are programmable and storable in recipe form in the controller 1037 memory. Bioreactor/pod recipes can be customized, recalled and run by the controller 1037 to optimize cell growth, preservation and recovery, particularly during isolation, activation, modification, expansion and washing processes that stress the cells.

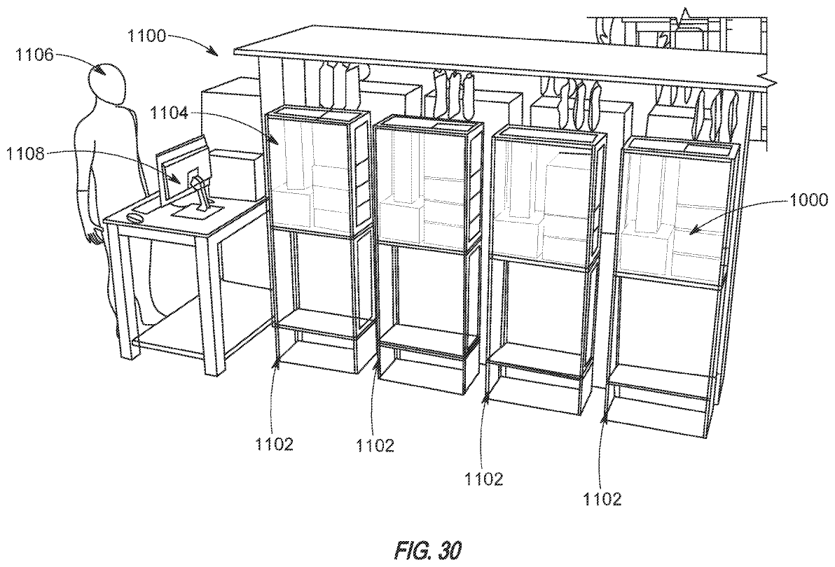

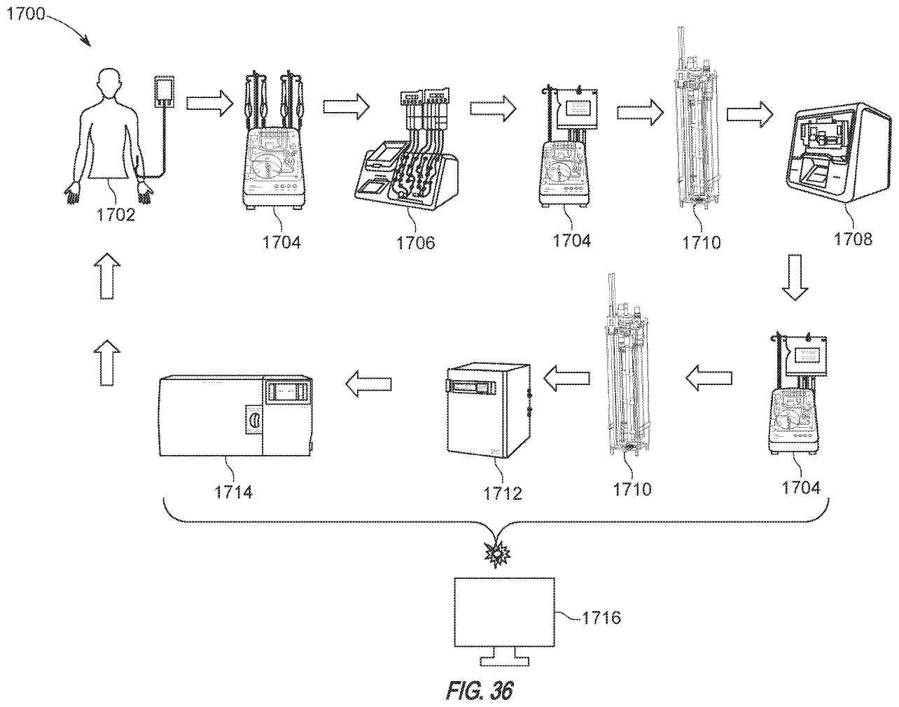

FIG. 30 is a bioreactor pod wall 1100 in accordance with example embodiments. The bioreactor pod wall 1100 includes two or more frames 1102 with two or more shelve sections 1104 that receive two of more bioreactor pods 1000. In other example embodiments, the bioreactor pod wall 1100 can include one frame housing or including several shelves. The bioreactor pods 1000 can be the same bioreactor pod 1000 described with respect to FIGS. 29A-B. An operator 1106 can interface with a terminal 1108 equipped with display and user interface to make inputs and receive outputs from each bioreactor pod 1000, to control the pod 1000 operations, and monitor pod 1000 parameters and outputs. Each bioreactor pod 1000 in the bioreactor pod wall 1100 can be associated with and contain biological components, such as cells or genetically modified cells, of a specific patient. After expansion, modification, secondary expansion and/or processing of the patient's cells as described with respect to FIG. 36, the cells can be reintroduced into the patient's body and administered as a therapeutic. In this way, the bioreactor pod wall 1100 can facilitate administration of cell therapy to several patients, and each bioreactor pod 1000 is specific, tailored and customized to a specific patient, the patient's disease and/or condition and a patient specific therapy.

FIGS. 31A-D depict front, back and cross-sectional views respectively, of a sensor assembly 1080 (or senor pack) in accordance with example embodiments. The sensor assembly 1080 is capable of coupling, protecting and operating a sensor at a top wall or other wall (e.g., sidewall) of the bioreactors disclosed herein. In example embodiments, the sensor assembly 1080 can be the sensor 1080 of FIGS. 29A-B that couples to port 1060A of FIGS. 29A-B. The sensor assembly 1080 includes a sensor hub 1202 with a port connector 1204 that can connect to the top bioreactor ports disclosed herein. In example embodiments, the port connector 1204 couples to port 1060A (or other bioreactor ports) via press fit, threaded connection, barb and cable tie connection or other connection. The sensor assembly 1080 includes a dip tube 1206 for inserting a resistance temperature detector 1208 (or other temperature sensor) with a metal tip 1210 for measuring temperature of the contents of the bioreactor 1010 (shown in FIGS. 29A-B). The sensor assembly 1080 also includes three foam sensors 1212 (or level sensors) to measure the volume, thickness, or content of foam in the bioreactor 1010 and to determine if antifoam measures should be deployed.

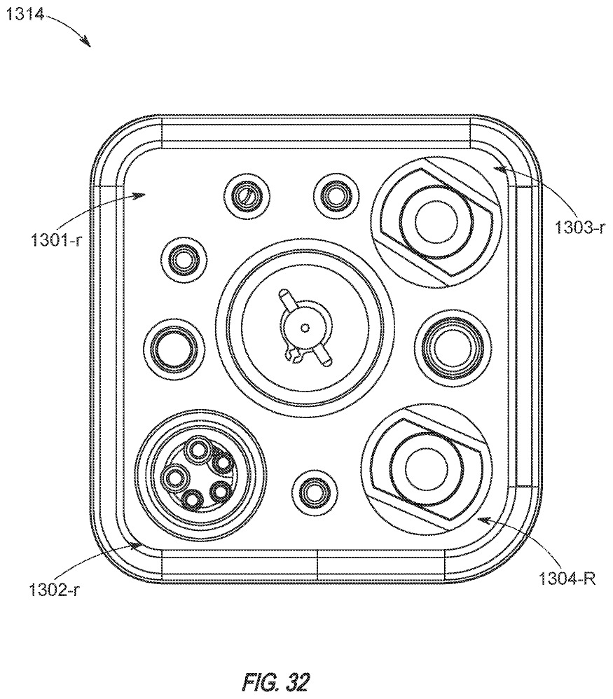

FIG. 32 is a top end wall 1314 (or top cap) of a bioreactor in accordance with example embodiments. The top end wall 1314 can be any of the top end walls attached to the bioreactors disclosed herein (e.g., top end walls 14, 368, 514, 714, 1026). In example embodiments, the top end wall 1314 can be the top end wall 1026 of the bioreactor 1010 described with respect to FIGS. 29A-B. The top end wall 1314 depicted in FIG. 32 can include any combination of components, ports, sensors, gas overlay assemblies and other components of top end walls 14, 368, 514, 714, 1026. In FIG. 32, the top end wall 1314 has four corners 1301-1304, each with a radius of curvature r, r, r, and R. Three of the corners 1301-1303 have a first radius of curvature r, and the fourth corner 1304 has a second and different radius of curvature R than the other three corners 1301-1304. In example embodiments, the fourth corner 1340 has a greater radius of curvature R than the other three corners 1301-1304 to create a poka-yoke feature that assures the top end wall 1314 is installed into/with the housing of the bioreactor (e.g., bioreactor 1010) in the proper orientation. The housings of the bioreactors disclosed herein can also have the same shape as the cap with three corners having a smaller radius of curvature than a fourth corner of the housings to properly fit and orient the top end wall 1314 into the housing.

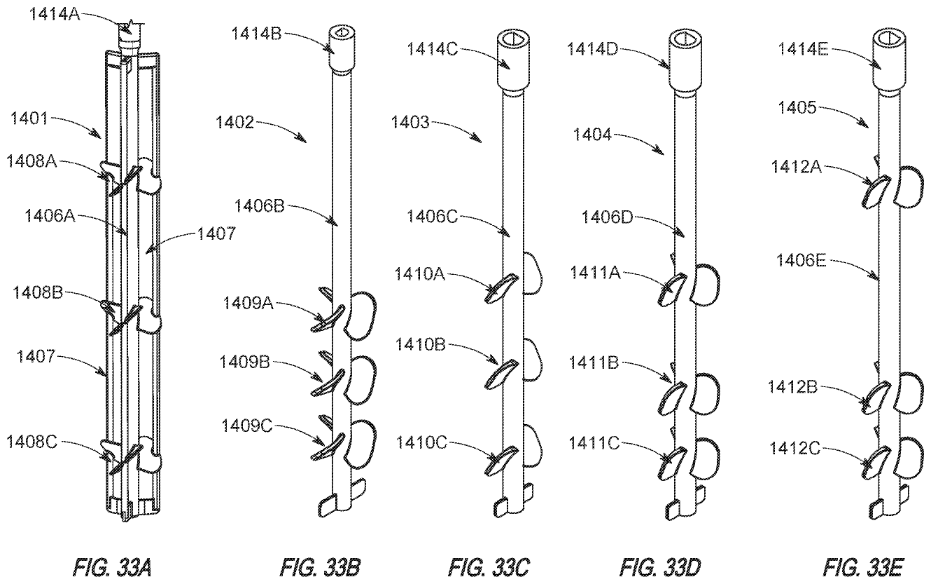

FIGS. 33A-E depict a series of impellers 1401-1405, respectively, that can be coupled to any of the bioreactors disclosed herein to mix the contents of the bioreactors. The impellers 1401-1405 are rotatably and movably disposed within the compartment of the bioreactor to mechanically mix biological components and other fluids within the compartment when the bioreactor is operating in the dynamic mode. In example embodiments, any of the impellers 1401-1405, are interchangeably mounted to bioreactors disclosed herein through any of the impeller mounting hubs disclosed herein (e.g., impeller mounting hubs 540, 740, 1022, 1500). In example embodiments, any of the impellers 1401-1405, are interchangeably mounted to the mounting hub 1022 of bioreactor 1010 described with respect to FIGS. 29A-B. Each impeller 1401-1405 has a primary drive shaft 1406A-E. Each impeller 1401-1405 also includes a top mount 1414A-E that couples to an impeller mounting hub (e.g., impeller mounting hubs 540, 740, 1022, 1500) to facilitate engagement with the top end wall of any of the disclosed bioreactors and motors described herein. Each impeller 1401-1405 has three sets of impeller blades 1408-1412.

Impeller 1401 has two supports 1407 spaced radially from the primary shaft 1406A that can add stability during mixing. Impeller 1401 also includes three sets of three blades (tri-blades) 1408A-C. Tri-blades 1408A and 1408C are spaced the same distance apart from tri-blade 1408B and are coupled to the supports 1407 and the primary shaft 1406A at three different sections of the shaft 1406A.

Impeller 1402 includes three sets of tri-blades 1409A-C. Tri-blades 1409A and 1409C are spaced the same distance apart from tri-blade 1409B and are coupled to the primary shaft 1406B and grouped near the bottom section of the shaft 1406B.

Impeller 1403 includes three sets of two blades (dual-blades) 1410A-C. Dual-blades 1410A and 1410C are spaced the same distance apart from dual-blade 1410B and are coupled to the primary shaft 1406C and grouped near the bottom section of the shaft 1406C, but the first dual-blade 1410A is positioned and coupled at a higher position on the primary shaft 1406C than tri-blade 1409A on primary shaft 1406B of impeller 1402.

Impeller 1404 includes three sets of tri-blades 1411A-C. The distance between tri-blades 1411A and 1411B is larger than the distance between tri-blades 1411B and 1411C, with tri-blades 1411B and 1411C coupled to the primary shaft 1406D and grouped further near the bottom section of the shaft 1406D.

Impeller 1405 includes three sets of tri-blades 1412A-C. The distance between tri-blades 1412A and 1412B is larger than the distance between tri-blades 1412B and 1412C, with tri-blades 1412B and 1412C coupled to the primary shaft 1406E and grouped further near the bottom section of the shaft 1406E. The first tri-blade 1412A is positioned and coupled at a higher position on the primary shaft 1406E than the first set of blades 1409A-1411A on primary shafts 1406B-D of impellers 1402-1404 respectively.

In example embodiments, the impellers 1401-1405 described herein include one to five sets of blades (1-5 blade sets), and each blade set can have from two to four blades per set (dual-blade, tri-blade or quad-blade). The blades can have a pitch of 0° to 45° from a horizontal plane perpendicular to the vertical axis (e.g., long axis) of the primary drive shafts 1406A-E when the impellers 1401-1405 are in the vertical orientation as depicted in FIGS. 33A-E. Each blade set can be coupled to the primary drive shafts 1406A-E at the same distance apart or at different distances apart. In example embodiments, the blade sets (e.g., 1408A-1412C) are spaced and coupled to the primary drive shafts 1406A-E at 5 mm to 50 mm apart between each blade set. Spacing between blade sets can be customized to achieve optimal mixing and cell growth.

FIGS. 34A-C depict perspective, cross sectional and partial top views of an impeller mounting hub 1500 respectively, in accordance with example embodiments. The impeller mounting hub 1500 includes a mounting shaft 1502 that runs and projects through the impeller mounting hub 1500. The mounting shaft 1502 has a bottom mount 1506 that can mount and couple to a drive shaft 1504 of an impeller in the compartment of the bioreactors disclosed herein (e.g., 510, 710, 1010). The bottom mount 1506 of the impeller mounting hub 1500 couples to a top mount 1516 of the drive shaft 1504 or other shafts (e.g., drive shafts 616, 716, 1018) of the impeller assemblies disclosed herein (e.g., impeller assemblies 550, 750, 1016) via press fit or other mechanical connection. In example embodiments, the bottom mount 1506 of the impeller mounting hub 1500 can include structures, protrusions, extensions or pins 1518 that are keyed to fit with (e.g., press fit) and couple to indentions 1520 in the top mount 1516 of the drive shaft 1504. Mounting hub 1500 can couple to a top mount of other drive shafts (e.g., drive shafts 616, 716, 1018) disclosed herein in similar keyed fashion. A portion of the mounting shaft 1502 engages with one or more bearing assemblies 1514A, 1514B to facilitate rotation of the mounting shaft 1502. A portion of the mounting shaft 1502 and one or more bearing assemblies 1514A, 1514B are contained and sealed within a hub cavity 1512 of the mounting hub 1500. The mounting hub 1500 can include a threaded portion 1508 to engage and couple to the top wall of the bioreactors and threaded bearing ports disclosed herein (e.g. threaded bearing ports 530, 730, 1030). A dust cover 1510 is located near or proximate to a terminal end of the impeller mounting hub 1500 to contain bearings 1514A-B and prevent dust or particles from entering the hub cavity 1512.

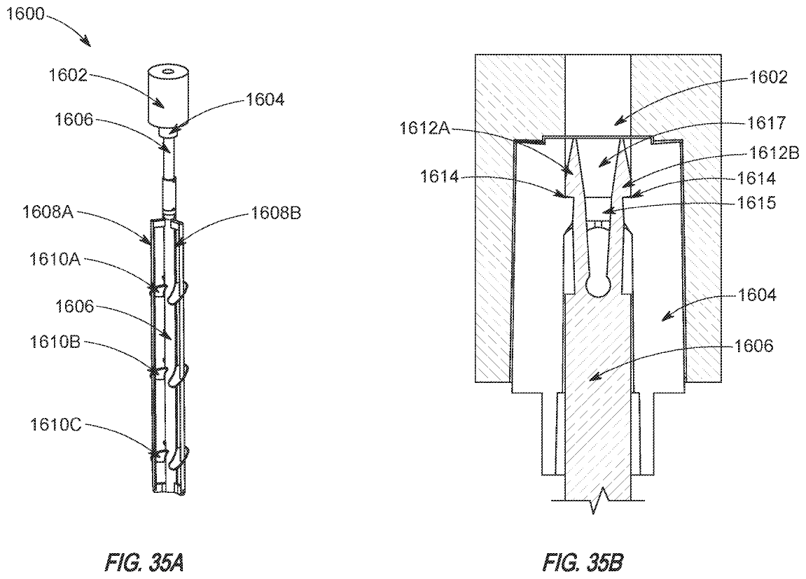

FIGS. 35A-B depict a perspective and cross-sectional views of an impeller assembly 1600 respectively, in accordance with example embodiments. The impeller assembly 1600 includes an impeller mounting hub 1602, a primary drive shaft 1606 that couples to the impeller mounting hub 1602, two supports 1608A-B, and three sets of tri-blades 1610A-C coupled to the primary drive shaft 1606 and supports 1608A-B at three different sections along the shaft 1606. The tri-blades 1610A-C can be spaced apart, grouped and configured in a variety of ways, for example, as described with respect to FIGS. 33A-E. The primary drive shaft 1606 can include two flexible, actuatable or bendable clips or prongs 1612A-B that can engage or snap-fit within a hub receiver 1604 of the impeller mounting hub 1602 to removably attach the shaft 1606 to the impeller mounting hub 1602. The prongs 1612A-B resiliently bend, actuate or compress inward into a compressed position as they are snap-fit or pressed fit within a narrow portion 1615 of the hub receiver 1604, and the prongs 1612A-B actuate, expand or spring back into an expanded position like a spring as they are snap-fit or press-fit through a wider portion 1617 of the hub receiver 1604. Flanges 1614 on the prongs 1612A-B prevent the prongs 1612A-B from being pulled through the narrower portion or passage 1615 of the impeller mounting hub 1602 unless a threshold force is applied or used to pull the prongs 1612A-B/drive shaft 1606 out of the hub receiver 1604. In other embodiments, the hub receiver 1604 does not have narrower and wider portions 1615,1617, and the prongs 1612A-B are simply compressed (or pinched) into a compressed position to fit the prongs into the hub receiver 1604 and allowed to resiliently spring back into an expanded position to retain the prongs 1612A-B and drive shaft 1606 in the hub receiver 1604. In this way, the prongs 1612A-B can quickly facilitate attachment and removal of the drive shaft 1606 (and other disclosed drive shafts herein) from the impeller mounting hub 1602.

FIG. 36 depicts an autologous cell therapy system and process flow 1700, including one or more bioreactors or bioreactor pods 1710 in accordance with example embodiments operating in static and/or dynamic modes. The autologous cell therapy system 1700 includes one or more equipment modules, including blood processing systems 1704, cell and bead processing systems 1706, gene editing systems 1708 (e.g., electroporation system), bioreactors/bioreactor pods 1710 (e.g., bioreactor pod 1000 and bioreactor 1010) disclosed herein, incubators 1712, freezers 1714 and associated automation software for controlling each equipment module with controller 1716. The autologous cell therapy system 1700 depicts one patient 1702 and includes three blood processing systems 1704 and two bioreactors/pods 1710, one gene editing system 1708, one incubator 1712 and one freezer 1714. The three blood processing systems 1704 and two bioreactors/pods 1710 depicted can be the same systems/bioreactors where cells and media are flowed and recycled or separate systems/bioreactors. Any combination of equipment modules described herein can be combined and customized to meet the operator and patient's needs.

In example embodiments, the autologous cell therapy system 1700 is dedicated to one patient 1702 and includes three blood processing systems 1704 and two bioreactors/pods 1710, one cell and bead processing system 1706, one electroporation system 1708, and one freezer 1714. The bioreactor/pod 1710 can operate as an incubator with the use of one or more top or bottom spargers, and therefore, the incubator 1712 is not necessary if a dual mode bioreactor 1710 is implemented.

At Step 1, the cell therapy process starts by drawing a blood sample from a patient 1702. The blood sample includes plasma, red blood cells, platelets and white blood cells or leukocytes.

At Step 2 of the cell therapy process, the blood sample is flowed or fed to a blood processing systems 1704 that can be used to separate the white blood cells (leukocytes) from the remainder of the patient's blood components. Example blood processing systems 1704 that can be used in the cell therapy system include the Gibco™ CTS™ Rotea™ Counterflow Centrifugation System and blood processing systems and methods disclosed in WO2018/204992 incorporated by reference in its entirety herein. The blood processing system 1704 can include a centrifuge (e.g., counterflow centrifuge) or other equipment used to separate the white blood cells (leukocytes) from the remainder of the patient's blood components in a leukapheresis process. The separated leukocytes can also be washed, reconstituted and/or suspended in fresh cell media or other media at the blood processing system 1704 prior to an expansion step at the bioreactor/pod 1710 or isolation/activation step at the cell and bead processing system 1706.

At Step 3 of the cell therapy process, the separated leukocytes are processed in a cell and bead processing system 1706 that includes at least a magnet and magnetic beads for processing cells. Exemplary cell and bead processing systems 1706 that can be used in the cell therapy system include the Gibco™ CTS™ DynaCellect™ Magnetic Separation System and the bead processing systems, methods, equipment and processing workflows disclosed in WO2022/081519 incorporated by reference in its entirety herein. The cell and bead processing system 1706 can be used to bind magnetic beads to specific cell types (e.g., stem cells, leukocytes in general, granulocytes, monocytes, total T cells, helper T helper cells, regulatory T cells, cytotoxic T cells, B cells, natural killer cells, thrombocytes, etc.) isolate, activate and wash the bound or unbound cells. For example, magnetic beads can be bound to target cell types via an antibody between the bead and cell that binds to the surface receptor of the cell by the antigen binding site of the antibody. A specific region of the antibody (e.g., Fc region of the antibody) is in turn linked to a linker, which connects the antibody to the magnetic bead. A magnet or magnet system can be used to attract and isolate the magnetic beads with the cells bound or after the cells are unbound from the magnetic bead via cleavage mechanisms described in detail in WO2022/081519 incorporated by reference in its entirety herein.

Target cells can be bound to magnetic beads, isolated, and activated within a bag or container of the cell and bead processing system 1706, described in detail in WO2022/081519 incorporated by reference in its entirety herein, in both positive and negative cell isolation processes. Example commercially available magnetic beads that can be used to isolate and activate target cells, include but are not limited to, DYNABEADS™ Human T-Expander CD3/CD28 (Thermo Fisher Scientific, cat. no.11141D), CTS™ DYNABEADS™ CD3/CD28 (Thermo Fisher Scientific, cat. no. 40203D), CTS™0 DYNABEADS™ Treg Xpander (Thermo Fisher Scientific, cat. no.46000D). In positive cell isolation processes, magnetic beads bind to the target cells and the bead/cell complexes are pulled to the magnet in the cell and bead processing system 1706. The supernatant is discarded, and the bead/cell complexes are washed with enzyme cleavage or other cleavage mechanism (described in detail in WO2022/081519 incorporated by reference in its entirety herein), resulting in activated target cells. In negative cell isolation, magnetic beads are bound to all unwanted or non-target cells, and non-target cells are attracted to the magnet in the blood processing system 1704 to deplete all the unwanted cells and retain unbound target cells in the separation bag.

Alternatively at Step 3 of the cell therapy process, the leukocytes can undergo cellular reproduction and/or expansion in one or more cell expansion processes in bioreactor/pod 1710 (e.g., bioreactor pod 1000 and reactor 1010) operating in static mode, dynamic mode or both modes for predetermined and/or alternating periods of time prior to isolation/activation at the cell and bead processing system 1706. Operating in static mode and dynamic mode (e.g., alternating periods of static and dynamic mode) during expansion of cells can relieve strain on the cells, promote optimal cell growth and preserve sensitive cells undergoing washing, isolation, activation and/or modification that cause cell stress and death during the cell therapy process 1700.

At Step 4 of the cell therapy process, target cells harvested from the cell and bead processing system 1706 can be washed, reconstituted and/or suspended in fresh cell media or other media and liquids (e.g., freezing media, water, buffer) at the bead processing system 1706 prior to an expansion step at the bioreactor/pod 1710. The target cells can also be fed to a blood processing system 1704 where they are separated, washed, reconstituted and/or suspended in fresh cell media or other media and liquids prior to an expansion step at the bioreactor/pod 1710. This Step 4 can also be eliminated.

At Step 5, target cells, that are isolated and/or activated in Step 3 in the cell and bead processing system 1706 and washed, reconstituted and/or suspended in fresh cell media in Step 4, undergo cellular reproduction and/or expansion in one or more cell expansion processes in bioreactor/bioreactor pod 1710 (e.g., bioreactor pod 1000 and reactor 1010) operating in static mode, dynamic mode or both modes for predetermined and/or alternating periods of time to facilitate optimal cell growth and preserve sensitive cells undergoing washing, isolation, activation and/or modification steps that subject the cells to stress.

At Step 6 of the cell therapy process, target cells can be flowed, fed or transferred to a gene editing system 1708 that edits, modifies or inserts a target DNA, RNA, protein, and/or other molecule into the target cells to generate a therapeutic result. Exemplary gene editing system 1708 that can be used in the cell therapy system 1700 include the CTS™ Xenon™ Electroporation System, Neon™ NxT™ Electroporation System, and the gene editing systems, methods, equipment and processing workflows disclosed in U.S. Publication Nos. 2021123009, 20230110090, and U.S. Pat No. D965170 incorporated by reference in their entirety herein.

At Step 7 of the cell therapy process, modified cells that were edited in the gene editing system 1708 can be flowed, fed or transferred to a blood processing system 1704 where the modified cells are separated, washed, reconstituted and/or suspended in fresh cell media or other media and liquids. This Step 7 can also be eliminated.

At Step 8 of the cell therapy process, cells modified in the gene editing system 1708 in Step 6 can be flowed, fed or transferred to in bioreactor/bioreactor pod 1710 to undergo cellular reproduction and/or expansion within bioreactor/bioreactor pod 1710 (e.g., bioreactor pod 1000 and reactor 1010) operating in static mode, dynamic mode or both modes for predetermined and/or alternating periods of time to facilitate optimal cell growth and preserve cells undergoing washing, isolation, activation and/or modification steps. Operating the bioreactor 1710 in alternating periods of static and dynamic mode during expansion of cells can relieve strain on the cells and promote optimal cell growth and preservation during the cell therapy process 1700.

In an optional Step 9, modified cells that were expanded in the bioreactor/bioreactor pod 1710 can be flowed, fed or transferred to an incubator 1712 for temporary storage or further expansion. In example embodiments, implementing the bioreactor pod or dual mode bioreactor 1710 eliminates the need for an incubator due to operability of the bioreactor/bioreactor pod 1710 in static mode with oxygenation of cells from the gas permeable membranes, sidewalls, top gas overlay assemblies and/or bottom spargers disclosed herein. The bioreactor 1710 can also be placed inside the incubator 1712 where gas is fed through a sidewall gas permeable membrane to further expand cells while operating in static mode with no impeller mixing as described previously.

At Step 10 of the cell therapy process, modified cells that were expanded in the bioreactor/bioreactor pod 1710 can be flowed, fed or transferred to a blood processing system 1704 where the modified and expanded cells are separated, washed, reconstituted and/or suspended in fresh cell media or freezing media in preparation for cold chain storage and processing and/or injection into the patient 1702 as a therapeutic.

At Step 11 of the cell therapy process, washed, reconstituted and/or suspended modified cells can be flowed, fed or transferred with freezing media and in a freezing bag to a freezer 1714 to freeze, transport and eventually thaw and administer the modified cells to the patient 1702 as a therapeutic.

The controller 1716 can include one or more processors, memory and software instructions run by the processor(s) to automate the cell therapy system, process 1700, and associated equipment modules, including one or more blood processing systems 1704, cell and bead processing systems 1706, gene editing systems 1708 (e.g., electroporation system), bioreactors/bioreactor pods 1710, incubators 1712, freezers 1714, and associated sensors and support modules. The controller can also include a client computer, display and user interface with operator inputs and system outputs for controlling the cell therapy system/process 1700. One or more equipment and process automation recipes can be stored in the memory of the controller 1716 and run by the operator via client computer and user interface. The period of time, number of times and sequence that the bioreactor/pod 1710 operates in dynamic and static mode during expansion steps and processes is programmable and storable in recipe form in the controller 1716 memory. Recipes can be customized, recalled and run by the controller 1716 to optimize cell growth, preservation and recovery.

The bioreactors disclosed herein have a number of unique benefits. For example, the bioreactors enable static cultivation of cells, i.e., without mixing, and a dynamic growth of cells, i.e., with light, heavy, low RPMs, high RPMs, intermittent, or continuous mixing, within the same bioreactor, thereby minimizing the delay, waste, and dangers associated with transferring cells between bioreactors or other equipment. The bioreactors are also unique in that they are configured to optimize production in both the static mode of operation and the dynamic mode of operation depending on the density, state, sensitivity, and application of the cells within the cell culture. In addition, bioreactors are easily rotated into horizontal, vertical or angled orientations when in static mode when the mixer/impeller is not operational and dynamic mode when the mixer/impeller is operational to further facilitate optimal cell growth. The dual mode bioreactors disclosed herein can be rotated into different orientations during cell expansion and during dynamic and static modes to expand while the bioreactor is in motion via rotation or translation. The bioreactors are also unique in that the relatively inexpensive components that contact the suspension during processing can be discarded/recycled after a single use while the relatively expensive components can be reused without any need for sterilizing. Other benefits and unique features also exist.

Any number of different cell culture media and culture media components may be used in conjunction with bioreactors provided herein. In many instances, cell culture media and culture media components will vary with the use of the cells being cultured and purpose of the cell expansion (e.g., use of expand cells, protein production, antibody production, etc.).

Cells (e.g., animal cells, such as mammalian cells) that may be expanded using the devices (e.g., bioreactors) and methods set out herein include immortalized cells (e.g., hybridoma cells) and primary cells (e.g., T cells, B cells, hepatocytes, etc.). Some types of cells that may be expanded using devices and methods set out herein include stem cells (e.g., induced pluripotent stem cells, embryonic stem cells, mesenchymal stem cells, etc.). Additional types of cells that may be expanded using devices and methods set out herein include immune system cells such as T cells (e.g., CD4+ T cells, CD8+ T cells, regulatory T cells, Th17 T cells, gamma delta T cells, memory T cells (e.g., central memory T cells), natural killer T cells, mucosal associated invariant T cells, etc.), natural killer (NK) cells, B cells, dendritic cells, antigen presenting cells, etc.

Some specific examples of cells that may be expanded using devices and methods set out herein include African green monkey cells (e.g., BSC cells), Hela cells, HepG2 cells, LLC-MK cells, CV-1 cells, COS cells, VERO cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, CHO cells, CHO-K1 cells, NS-1 cells, MRC-5 cells, WI-38 cells, 3T3 cells, 293 cells, Per.C6 cells and chicken embryo cells. In some instances, a CHO cell line or one or more of several specific CHO cell variants optimized for large-scale protein production (e.g., CHO-K1) is expanded.

T cells, for example, may be expanded in a number of different culture media, including X-VIVO 15™ (Lonza, cat. no. BE02-060Q) and OPTMIZER™ CTS™ SFM, AIM-V, and RPMI 1640 (Thermo Fisher Scientific, cat. nos. A1048501, 0870112DK, 11875119). Further, T cells may be expanded with serum or without serum. Additional, T cells may be expanded with a serum replacement, such as CTS™ Immune Cell Serum Replacement (ICSR) (Thermo Fisher Scientific, catalog number A2596101).

T cells may be activated before, during, and/or after expansion. For example, T cells may be activated in a bioreactor during expansion. By way of further example, T cells may be activated through contact with anti-CD3 and anti-CD28 antibodies. Such antibodies may be bound to one of more solid support (e.g., beads). Further, T cells may also be contacted with one or more cytokine (e.g., interleukin-2, etc.) before, during, and/or after expansion. Thus, provided herein are methods for expanding T cells in a bioreactor.

Culture media that may be used in conjunction with devices and methods set out herein include Eagle's MEM (minimal essential media), Ham's F12, F-12 K, Dulbecco's, Dulbecco's Modified Eagle Medium, DMEM/Ham's F12 1:1, Trowell's T8, A2, Waymouth, Williams E, MCDB 104/110, RPMI-1640 Medium, RPMI-1641 Medium, Iscove's Modified Dulbecco's Medium, McCoy's 5 A, Leibovitz's L-15, EX-CELL™ 300 Series (JRH Biosciences, Lenexa, KS), protamine-zinc-insulin media. Media may contain serum or be serum free.

Cells may be expanded in “fed-batch cell culture” processes. “Fed-batch culture” refers to a batch culture wherein the animal cells and culture medium are supplied to the culturing vessel initially and additional culture nutrients are fed continuously or in discrete increments, to the culture during culturing, with or without periodic cell and/or product harvest before termination of culture. Fed-batch culture includes “semi-continuous fed-batch culture” wherein periodically whole culture (which may include cells and media) is removed and replaced by fresh medium.

Fed-batch culture is distinguished from simple “batch culture” whereas all components for cell culturing (including the animal cells and all culture nutrients) are supplied to the culturing vessel at the start of the culturing process in batch culture.

Cells may also be expanded in perfusion processes. In perfusion culturing, the cells are restrained in the culture by (e.g., filtration) and the culture medium is continuously or intermittently introduced and removed from the culturing vessel.

Some aspects of compositions and methods set out herein relate to access of cells being expanded to oxygen and the removal of carbon dioxide. It is generally desirable for these cells to have ready access to oxygen with the efficient removal of carbon dioxide. Along these lines, typically cells expanded as set out herein will be present in a bioreactor where one or both of these parameters may be coordinated for efficient cell expansion. The O₂ concentration in such bioreactors may be between 15% and 25% (e.g., from about 15% to about 24%, from about 17% to about 25%, from about 18% to about 25%, from about 20% to about 25%, from about 22% to about 25%, from about 23% to about 25%, etc.). Further, the CO₂ concentration in such bioreactors may be between 2% and 7%.

In many instances, as previously discussed, gas exchange will be facilitated by the use of a gas permeable membrane in contact with the culture media. Such membranes may be located at one or more side, the top and/or the bottom of a bioreactor and allow both for O₂ to enter culture media and for CO₂ to leave the culture media. In some instances, gas permeable membrane used in bioreactors and methods set our herein made be composed of or comprise gas permeable silicone (e.g., dimethyl silicone) and/or may be between 0.001 and 0.01 (e.g., from about 0.005 to about 0.007, from about 0.002 to about 0.007, from about 0.003 to about 0.007, from about 0.005 to about 0.009, from about 0.004 to about 0.008, etc.) inches in thickness.

In some instances, it may be desirable for a glutamine source to be present in the culture medium. When this is the case, then the glutamine source may be one that will not form substantial amounts of ammonia. One example of such a glutamine source is an L-alanyl-L-glutamine dipeptide. When present, such a glutamine reagent may be present at a concentration of between from about 1 mM to about 20 mM (e.g., from about 2 mM to about 20 mM, from about 5 mM to about 18 mM, from about 10 mM to about 20 mM, from about 8 mM to about 27 mM, etc.).

Further, while incubation temperatures for the expansion of cells, including immune cells (e.g., NK cells, T cells, B cells, and/or APCs) may vary but mammalian cells will typically be cultured at temperatures between 34° C. and 40° C. (e.g., 37° C.).

Cells being expanded may be contacted with one or more chemokine or cytokine, depending on the cell type that is being expanded. Chemokine and cytokine that may be used include Interleukin-1α, Interleukin-2, Interleukin-4, Interleukin-1β, Interleukin-6, Interleukin-12, Interleukin-15, Interleukin-18, Interleukin-21, and Transforming growth factor ß1.

Bioreactors set out herein may also be used to incubate cells in conjunction with one or more processes related to the induction of materials (e.g., DNA, RNA, proteins, protein/nucleic acid/complexes, etc.) into cells (e.g., eukaryotic cells such as mammalian cells). Material introduction processes include transduction (e.g., viral transduction) and transfection. Exemplary transduction and transfection methods include calcium phosphate transfection, liposomal transfection, nucleofection, sonoporation, transfection through heat shock, magnetifection and electroporation. Vectors used in exemplary viral transduction methods that may be used in the methods described herein include, but are not limited to retroviral (e.g., lentiviral), adenoviral, and adeno-associated viral vectors.

The method by which materials are introduced will vary with a number of factors, including the materials to be introduced into the cells. For example, electroporation will generally be more suitable for introducing guide RNA/Cas9 complexes into cells than by lentiviral transduction. Further, viral transduction may be more suitable than electroporation when nucleic acids (e.g., nucleic acid encoding chimeric antigen receptors) are sought to be introduced into cells and there is a desire to maintain high cell viability.

Electroporation is a non-viral process that may be used to introduce a wide range of materials into cells. Electroporation involves the application of an electric field to cells resulting in the cell membranes being compromised, thereby allowing for the cellular uptake delivery of exogenous materials.

A considerable amount of work has been done on mechanistic theories related to the response of cell membranes to electric field pulses that rapidly increase the transmembrane voltage, Um(t), of cell membranes to a value where cell membrane porosity dramatically rises (see Weaver et al., Bioelectrochemistry 87:236-243 (2012)). The change in membrane porosity is believed to be caused by pore formation. Thus, materials uptake is believed to be mediated by the induction of pore formation in cell membranes.

Large electric field pulses used for electroporation can kill cells either through heating or without heating being the main cause. Two non-heat killing mechanisms are believed to be via induction of apoptosis or necrosis. Further, high strength electric field cell killing is believed to be more by apoptosis, while low strength electric field cell killing is believed to be more by necrosis. Thus, it is generally desirable to adjust electrical field conditions, as well as other parameters, such that high cell viability is maintained, regardless of the cell death mechanism.

Bioreactors set out herein may be used to maintain the viability of cells by operating in static mode or low mixing RPMs (e.g., below 40 RPMs or below 10 RPMs) in dynamic mode, after these cells have been exposed to electric fields (e.g., electroporation). Exemplary methods of maintaining cell viability include exposing cells to an electric field followed by incubation of these cells while operating the bioreactors set out herein in static mode. This incubation may be in culture media or a medium designed to allow for the cells to remain in low metabolic state during the incubation period (e.g., an osmotically stabilizing solution containing minimally sufficient nutrients to prevent significant decrease in cell viability).

In many instances, the cells will be incubated in the bioreactor after electroporation for a fixed incubation period (e.g., from about 30 minutes to about 21 days, from about 30 minutes to about 3 hours, from about 30 minutes to about 5 hours, from about 30 minutes to about 10 hours, from about 30 minutes to about 15 hours, from about 30 minutes to about 20 hours, from about 30 minutes to about 24 hours, from about 30 minutes to about 40 hours, from about 1 hour to about 5 hours, from about 1 hour to about 10 hours, from about 1 hour to about 24 hours, from about 5 hours to about 15 hours, from about 5 hours to about 24 hours, from about 10 hour to about 30 hours, from about 24 hours to about 21 days, from about 2 days to about 21 days, from about 5 days to about 21 days, from about 8 days to about 21 days, from about 24 hours to about 48 hours, from about 24 hours to about 72 hours, etc.), where no mechanical mixing (static mode) or low level mechanical mixing occurs (impeller RPMs less or equal to than 10 in dynamic mode (e.g., from about 0.1 to about 10, from about 0.5 to about 10, from about 0.8 to about 10, from about 1.0 to about 10, from about 2.0 to about 10, from about 3.0 to about 10, from about 4.0 to about 10, from about 5.0 to about 10, from about 0.1 to about 1, from about 0.1 to about 0.8, from about 0.1 to about 0.6, from about 0.5 to about 1, from about 0.3 to about 1, from about 0.3 to about 0.8, etc. RPMs)) to allow for cells to recover from electroporation effects, referred to herein as a “recovery incubation period”.

Recovery incubation periods may alternate between static mode and dynamic mode. By way of example, a bioreactor may be operated in static mode for a period of time and then may be operated in dynamic mode for a period of time. One exemplary set of conditions would be static mode for 30 minutes, followed by dynamic mode for 30 minutes with an impeller RPM of 2 in dynamic mode. Using the above exemplary set of conditions for illustration, the static mode to dynamic mode ratio would be 1:1. In some instances, the ratio of static mode to dynamic mode may be from 1:10 to 10:1 (e.g., 1:1 to 1:10, 1:1 to 1:5, 1:1 to 1:3, 10:1 to 1:1, 1:10 to 5:1, 10:1 to 3:1, 1:5 to 5:1, 1:2 to 2:1, etc.).

Additionally, the number of alternate between static mode and dynamic mode (or mixing “pulses”) may be from 1 to 1,000 (e.g., from about 10 to about 1,000, from about 20 to about 1,000, from about 100 to about 1,000, from about 10 to about 500, from about 10 to about 250, from about 10 to about 150, from about 30 to about 250, from about 50 to about 500, etc.) over the course of the recovery period or the entire post electroporation culture period.

Further, O₂ and CO₂ concentrations in the bioreactor may be adjusted during the recovery incubation period to maintain high cell viability.

Cells (e.g., mammalian cells) may be incubated in a bioreactor set out herein before, after, and/or during exposure to an electric field (e.g., electroporation). For example, cells may be cultured in a bioreactor set out herein, may then be removed from the bioreactor, and then may be reintroduced into the same or different bioreactor. As an alternative to the above, the cells may be electroporated within the bioreactor. Further, cells (e.g., T cells) may be obtained from a patient, subjected to electroporation, then introduced into a bioreactor set out for cultivation. These cultivated T cells may then be reintroduced into the patient.

One method for increasing viability of cells exposed to an electric field (e.g., electroporated) is through preincubation of these cells with high density lipoprotein (HDL). By way of example, mammalian cells (e.g., T cells) may cultivated for three days in CTS OPTMIZER™ (Thermo Fisher Scientific, cat. no. A37050-01) with 6 mg/l HDL prior to electroporation. Methods such as this are set out in U.S. Patent Publication No. 2021/0024882, published Jan. 28, 2021, entitled “COMPOSITIONS AND METHODS FOR ENHANCING CELL CULTURE”, the entire disclosure of which is incorporated herein by reference. Further, such preincubation may be performed in a bioreactor set out herein. Thus, provided herein are methods in which cells are cultivated with HDL for a period of time (e.g., from about 1 day to about 5 days, from about 2 days to about 4 days, etc.), followed by the introduction of one or more materials into these cells.

Methods set out herein thus include those comprising materials that are introduced by electroporation into cells and the cells are then incubated in a bioreactor set out herein in static, dynamic and/or alternating modes of cell expansion, preservation, and processing within a single bioreactor. Further, such methods may involve the preincubation of cells with HDL.

Cells may be transduced with viral vectors in a bioreactor set out herein. Methods for such transduction include contacting these cells in a bioreactor with one or more viral vectors. In many instances, these viral vectors will contain nucleic acids comprising a nucleic acid region for insertion into intracellular nucleic acid (e.g., a chromosome of the cell the nucleic acid region is introduced into) and/or encode a protein for intracellular expression (e.g., Cas9 protein, chimeric antigen receptor (CAR), etc.).

Provided herein are methods for the culture of, introduction or materials into, and/or the engineering of cell. By way of example, activated T cells may be expanded in bioreactor set out herein. A lentiviral vector encoding a CAR may then be introduced into the bioreactor, under conditions which allow for the lentiviral vector enters the T cells to generate CAR-T cells. The CAR-T cells may then be further expanded in the bioreactor.

In many instances, viral (e.g., lentiviral) transduction will occur in bioreactors set out herein for a time period (e.g., from about 1 hour to about 2 days, from about 3 hour to about 1 days, from about 5 hours to about 2 days, etc.) with no mechanical mixing (static mode) or minimal mechanical mixing occurs (equal to or less than 10 impeller RPMs in dynamic mode). The resulting transduced T cells may then be subjected to recovery incubation period conditions similar to that set out above for electroporation. Further, viral particles may be removed from the bioreactor during the recovery incubation period.

### Example 1: Conditions for the Culture of T Cells

T Cell Isolation: Primary human T cells from normal donors are negatively isolated from PBMCs with a DYNABEADS™ UNTOUCHED™ Human T Cells kit (Thermo Fisher Scientific, cat. no. 11344D), which can be used to remove cells having the following markers: CD14, CD16 (a and b), CD19, CD36, CD56, CD123 and CD235A (e.g., B cells, NK cells, monocytes, platelets, dendritic cells, granulocytes, and erythrocytes).

Media: Basal growth media included X-VIVO 15™ (Lonza, cat. nos. BE02-060Q), OPTMIZER™ CTS™ SFM, AIM-V SFM, and RPMI 1640 (Thermo Fisher Scientific, cat. nos. A1048501, 0870112DK, 11875119) are used for T cell expansion. Media are supplemented with 5% human AB serum (hABs) (Gemini Bio-Products) or 2.5% CTS Immune Cell Serum Replacement (Thermo Fisher Scientific, cat. no. A2596101).

Activation: T cells are activated with DYNABEADS™ Human T-Expander CD3/CD28 (Thermo Fisher Scientific, cat. no. 11141D) at a ratio of 3 beads per T cell in the presence of 100 IU/ml of rIL-2 (Thermo Fisher Scientific, cat. no. PHC0021).

Expansion: T cells are maintained at 5×10⁶ cells/ml and counted on days 3, 5, 7, and 10 using a Beckman-Coulter Vi-Cell analyzer. In addition, rIL-2 is replenished on these same days. Cell growth is expressed as fold expansion over time. Media is exchanged on days 5 and 7 and 100 IU/ml of rIL-2 is replenished on days 3, 5, and 7. The culture temperature is 37° C. The CO₂ concentration of the culture medium is maintained at about 5 percent. The O₂ concentration of the culture medium is maintained at about 17-21%.

Endpoints: Cellular phenotype is assessed on day 10 by staining T cells with anti-CD3-Pacific Orange, anti-CD4-FITC, anti-CD8-Pacific Blue, anti-CD62L-APC, and anti-CCR7-PE (Thermo Fisher Scientific, cat. nos. CD0330, 11-0041-82, MHCD0828, 17-0621-82, 12-1971-82). To assess cytokine production (data not shown), DYNABEADS™ Human T-Expander CD3/CD28 are removed from the cultures on day 10, the T cells are washed, and rested them overnight in fresh medium. 2.5 million T cells are seeded at 1×10⁶ T cell/mL and re-stimulated with Human T-Expander CD3/CD28 at a 1:1 bead to cell ratio and incubated for 24 hours. Supernatants are collected and processed for analysis with INVITROGEN™ Cytokine Human Magnetic 35-Plex Panel for LUMINEX™ (Thermo Fisher Scientific, cat. no. LHC6005M).

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1.-91. (canceled).

92. A bioreactor pod comprising:
a pod base;
a bioreactor removably coupled to the pod base;
a first pod module for transporting a first fluid to and from the bioreactor and regulating a flow rate of the first fluid to and from the bioreactor;
a display comprising a user interface for transmitting operator inputs and receiving process parameter outputs associated with the bioreactor; and
a controller comprising a memory operatively associated with the processor for controlling a component associated with the bioreactor or a component associated with the pod module.

93. The bioreactor pod as recited in claim 92, wherein the bioreactor is a dual mode bioreactor that can culture cells in a cell culture media in a static mode with no mixing of the cell culture media and a dynamic mode with mixing of the cell culture media.

94. The bioreactor pod as recited in claim 92, wherein the bioreactor comprises a top end wall with a first top port and a bioreactor base with a first bottom port.

95. The bioreactor pod as recited in claim 94, further comprising a gas overlay assembly coupled to the first top port and a sparger coupled to the first bottom port.

96. The bioreactor pod as recited in claim 94, wherein the top end wall further comprises a second top port and a first sensor assembly coupled to the second top port for measuring a process parameter.

97. The bioreactor pod as recited in claim 96, wherein the process parameter comprises a pressure within the bioreactor, a temperature of the cell culture media, a foam content within the bioreactor, a glucose content of the cell culture media, a pH of the cell culture media, a dissolved oxygen content of the cell culture media, a CO₂ content of the cell culture media, or cell density of the cell culture media.

98. The bioreactor pod as recited in claim 92, wherein the first pod module is detached from the pod base.

99. The bioreactor pod as recited in claim 92, wherein the first pod module is a first pump module for pumping the first fluid to the bioreactor and the first fluid is a liquid comprising cells.

100. The bioreactor pod as recited in claim 99, wherein the first pump module comprises two pumps.

101. The bioreactor pod as recited in claim 92, further comprising a second pod module for transporting a second fluid to the bioreactor and regulating a flow rate of the second fluid to the bioreactor.

102. The bioreactor pod as recited in claim 101, wherein the second pod module is detached from the pod base.

103. The bioreactor pod as recited in claim 101, wherein the second pod module is a second pump module for pumping the second fluid to the bioreactor and the second fluid is the cell culture media.

104. The bioreactor pod as recited in claim 103, wherein the second pump module comprises two pumps.

105. The bioreactor pod as recited in claim 95, further comprising a third pod module for transporting a third fluid to the bioreactor and regulating a flow rate of the third fluid to the bioreactor.

106. The bioreactor pod as recited in claim 105, wherein the third pod module is detached from the pod base.

107. The bioreactor pod as recited in claim 105, wherein the third pod module is a mass flow controller and the third fluid is oxygen gas.

108. The bioreactor pod as recited in claim 105, wherein the third pod module is fluidically connected to the gas overlay assembly and the sparger to transport oxygen gas into the bioreactor through the gas overlay assembly and the sparger.

109. The bioreactor pod as recited in claim 92, wherein the controller is contained within the bioreactor base.

110. The bioreactor pod as recited in claim 94, wherein the pod base comprises a heating element for heating the bioreactor base.

111. The bioreactor pod as recited in claim 92, wherein the bioreactor comprises an impeller assembly comprising:
an impeller shaft;
three tri-blades spaced vertically apart and coupled to the impeller shaft; and
an impeller mounting hub.

112.-126. (canceled).
